(19) United States
(12) Patent Application Publication
(10) Pub. No.: US 2017/0321240 A1
(43) Pub. Date: Nov. 9, 2017

(54) GLYCOSYLATION MUTANTS FOR PRODUCING GALACTOSE-FREE AND FUCOSE-FREE POLYPEPTIDES

(71) Applicant: AGENCY FOR SCIENCE, TECHNOLOGY AND RESEARCH, Singapore (SG)

(72) Inventor: ZHIWEI SONG, Singapore (SG)

(73) Assignee: AGENCY FOR SCIENCE, TECHNOLOGY AND RESEARCH, Singapore (SG)

(21) Appl. No.: 15/522,206

(22) PCT Filed: Oct. 27, 2015
(86) PCT No.: PCT/SG2015/050411

§ 371 Date: Apr. 26, 2017

(30) Foreign Application Priority Data
Oct. 27, 2014 (SG) 10201406993Q

(51) Int. Cl. C12P 21/00; C12N 5/071; C07K 16/32; C07K 14/505; C07K 14/705; C07K 16/30

(52) U.S. Cl. CPC C12P 21/005; C07K 14/705; C07K 16/30; C07K 16/32; C07K 14/505; C12N 5/0682; C07K 2317/14; C07K 2317/41; C07K 2319/30; C07K 2319/91; C12N 2500/90; C12N 2510/02; C07K 2317/732

(57) ABSTRACT

We describe a method of reducing batch-to-batch variation or increasing homogeneity between batches in the production of a recombinantly expressed polypeptide. The method comprises expressing the polypeptide in a Chinese hamster ovary (CHO) cell comprising reduced UDP-galactose transporter activity compared to a wild-type CHO cell. In some embodiments, the CHO cell further comprises reduced GDP-fucose transporter activity.

### FIELD

This invention relates to the fields of medicine, cell biology, molecular biology and genetics. This invention relates to the field of medicine.

### BACKGROUND

A number of expression systems using different culture conditions have been employed to express polypeptides. One example is the use of Chinese Hamster Ovary Cells for polypeptide expression.

For example, recombinant human IgG1 antibodies have been successfully used as therapeutic drugs to target malignant cells in cancer patients.

Although the integrity of polypeptide chains seems to be largely unchanged in these systems, significant changes in glycosylation have been noticed.

It is known that glycosylation varies with cell line and animal species. Glycosylation of antibodies also varies with culture conditions. Therefore, glycosylation of antibodies expressed under different cell culture conditions will vary from batch to batch. Depending on the mechanism of action for each expressed polypeptide, these variations will affect product quality as well as product potency.

Antibodies produced in CHO cells mainly contain core fucosylated biantennary complex oligosaccharides terminated with 0, 1, or 2 Gal residues (FIG. 8). These are commonly designated as G0, G1, and G2 structures.

The N-glycans attached to the IgG1 antibodies produced by CHO cells are mainly core-fucosylated complex type containing zero or one galactose residue (G0F or G1F), and a small amount of G2F.

The relative proportions of G0, G1, and G2 oligosaccharides vary from batch to batch and are dependent on cell culture conditions. Depending on the mechanism of action for a given therapeutic antibody, variations in G0, G1, and G2 glycans affect product quality and bioactivity.

Therefore, the ratio of these components in the total N-glycans can vary dramatically between different products. This variation represents a serious regulatory concern because of quality assurance. Regulatory agencies are paying close attention to glycosylation variations and their impact on product quality.

There is therefore a need in the art for a means to address these issues at the early phases of development. There is also a requirement for the impact of glycosylation on a particular antibody product to be addressed at an early stage.

### SUMMARY

According to a 1^{st} aspect of the present invention, we provide a method of reducing batch-to-batch variation or increasing homogeneity between batches in the production of a recombinantly expressed polypeptide. The method may comprise expressing the polypeptide in a Chinese hamster ovary (CHO) cell comprising reduced UDP-galactose transporter activity compared to a wild-type CHO cell.

The CHO cell may comprise a loss of function mutation in a UDP-galactose transporter gene (Slc35a2).

The CHO cell may comprise a loss of function mutation in a UDP-galactose transporter gene (Slc35a2) comprising a T insertion at position 955 of the Slc35a2 open reading frame.

The CHO cell may comprise a loss of function mutation in a UDP-galactose transporter gene (Slc35a2) such that both alleles of a UDP-galactose transporter gene comprise a loss of function mutation.

The CHO cell may be comprised in a CHO-gmt2 cell line (deposited on 21 Oct. 2014 at the American Type Culture Collection (ATCC), P.O. Box 1549, Manassas, Va. 20108, United States of America under the Budapest Treaty as accession number PTA-121624).

The CHO cell may further comprise reduced GDP-fucose transporter activity.

The CHO cell may be capable of expressing recombinant antibodies. The CHO cell may be capable of expressing tumour and/or cancer-targeting antibodies. Such antibodies may have enhanced antibody-dependent cellular cytotoxicity (ADCC).

The CHO cell may comprise a loss of function mutation in a GDP-fucose transporter gene (Slc35c1 or Slc35c2).

The CHO cell may comprise a loss of function mutation in a GDP-fucose transporter gene (Slc35c1 or Slc35c2) in which both alleles of a GDP-fucose transporter gene comprise a loss of function mutation.

The loss of function mutation may comprise a 3-nucleotide (GTA) insertion or a 4-nucleotide insertion at position 411 of Slc35c1.

The CHO cell may be comprised in a CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

Homogeneity may be assayed by detecting the number of peaks in a liquid chromatogram of N-glycans of a recombinant polypeptide expressed by a CHO cell comprising reduced UDP-galactose transporter activity. This may be compared to a control comprising a liquid chromatogram of N-glycans of recombinant polypeptide expressed by wild type CHO-K1.

An increase in homogeneity may be detected as a reduction in the number of peaks as assayed in the method set out above, for example to one peak compared to 3 peaks in the control.

Homogeneity may further be assayed by measuring the area under the peak of the major product (i.e., G0F) in a liquid chromatogram of N-glycans of a recombinantly expressed polypeptide as a percentage of the total area under the curve.

Batch to batch variation may be assayed by determining the ratio of the respective areas under the peaks of the products in a liquid chromatogram of N-glycans of a recombinantly expressed polypeptide.

Batch-to-batch variation may be reduced and/or homogeneity increased by 5% or more. Batch-to-batch variation may be reduced and/or homogeneity increased by 10% or more. Batch-to-batch variation may be reduced and/or homogeneity increased by 15% or more. Batch-to-batch variation may be reduced and/or homogeneity increased by 20% or more. Batch-to-batch variation may be reduced and/or homogeneity increased by 25% or more. Batch-to-batch variation may be reduced and/or homogeneity increased by 30% or more. Batch-to-batch variation may be reduced and/or homogeneity increased by 35% or more. Batch-to-batch variation may be reduced and/or homogeneity increased by 40% or more. Batch-to-batch variation may be reduced and/or homogeneity increased by 45% or more. Batch-to-batch variation may be reduced and/or homogeneity increased by 50% or more. Batch-to-batch variation may be reduced and/or homogeneity increased by 55% or more. Batch-to-batch variation may be reduced and/or homogeneity increased by 60% or more. Batch-to-batch variation may be reduced and/or homogeneity increased by 65% or more. Batch-to-batch variation may be reduced and/or homogeneity increased by 70% or more. Batch-to-batch variation may be reduced and/or homogeneity increased by 75% or more. Batch-to-batch variation may be reduced and/or homogeneity increased by 80% or more. Batch-to-batch variation may be reduced and/or homogeneity increased by 85% or more. Batch-to-batch variation may be reduced and/or homogeneity increased by 90% or more. Batch-to-batch variation may be reduced and/or homogeneity increased by 95% or more.

Batch-to-batch variation or homogeneity or both may be measured using mass spectrometry. It or they may also be measured using liquid chromatography.

The liquid chromatography may comprise liquid chromatography mass spectrometry (LC-MS) or ultra-high performance liquid chromatography (UHPLC).

The recombinantly expressed polypeptide may comprise predominantly G0F. It may comprise G0F with low levels or no G1F or G2F N-glycans.

The polypeptide may comprise erythropoietin-Fc fusion polypeptide (EPO-Fc). The polypeptide may comprise MUC1-Fc fusion polypeptide. The polypeptide may comprise an antibody. The polypeptide may comprise anti-HER2 antibody (Herceptin). The polypeptide may comprise anti-CD20 antibody (Rituxan or GA101). The polypeptide may comprise IgG1. The polypeptide may comprise IgG2. The polypeptide may comprise IgG3. The polypeptide may comprise IgG4.

The CHO cell or cell line may be adapted to suspension culture in a serum-free medium.

There is provided, according to a 2^{nd} aspect of the present invention, a method of production of a recombinantly expressed polypeptide with reduced batch-to-batch variation or increased homogeneity between batches. The method may comprise a method as set out above.

The method may further comprise allowing the polypeptide to be expressed from the CHO cell or a descendent thereof and purifying the polypeptide.

We provide, according to a 3^{rd} aspect of the present invention, use of a Chinese hamster ovary (CHO) cell which has reduced UDP-galactose transporter activity compared to a wild-type cell, in a method of expressing recombinant polypeptide with reduced batch-to-batch variation or increased homogeneity between batches, or both.

As a 4^{th} aspect of the present invention, there is provided a method comprising expressing a recombinant polypeptide in a Chinese hamster ovary (CHO) cell which has reduced UDP-galactose transporter activity compared to a wild-type cell and detecting reduced batch-to-batch variation or increased homogeneity between batches, or both.

We provide, according to a 5^{th} aspect of the present invention, a recombinant polypeptide produced by a method set out above.

The present invention, in a 6^{th} aspect, provides a plurality of batches of recombinant polypeptide each as set out above. The batch-to-batch variation between the batches may be reduced or homogeneity between the batches may be increased, when compared to batches of recombinant polypeptide produced by wild-type CHO cells.

In a 7^{th} aspect of the present invention, there is provided a method of producing a Chinese hamster ovary (CHO) cell suitable for recombinant polypeptide expression with reduced batch-to-batch variation or increased homogeneity, or both. The method may comprise reducing UDP-galactose transporter activity in or of the cell. This may be achieved by introducing a loss of function mutation in a UDP-galactose transporter gene (Slc35a2).

According to an 8^{th} aspect of the present invention, we provide a Chinese hamster ovary (CHO) cell comprising reduced UDP-galactose transporter activity and reduced GDP-fucose transporter activity.

The cell may be capable of expressing recombinant antibodies. The recombinant antibodies ma comprise tumour/cancer-targeting antibodies with enhanced antibody-dependent cellular cytotoxicity (ADCC).

The cell may exhibit reduced batch-to-batch variation or increased homogeneity between batches.

The CHO cell may comprise a loss of function mutation in a UDP-galactose transporter gene (Slc35a2). The CHO cell may comprise a loss of function mutation in a GDP-fucose transporter gene (Slc35c1 or Slc35c2).

The CHO cell may comprise a T insertion at position 955 of the Slc35a2 open reading frame and a 3-nucleotide (GTA) insertion. The CHO cell may comprise a 4-nucleotide insertion at position 411 of Slc35c1.

The CHO cell may be comprised in a CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

The CHO cell may be or may have been adapted to suspension culture in a serum-free medium.

We provide, according to a 9^{th} aspect of the invention, use of a CHO cell as set out above in a method of expression of a recombinant polypeptide.

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 is a drawing showing isoelectric focusing analysis of EPO rescue assay in CHO-K1 and the glycosylation mutants. CHO-K1 was transiently transfected with EPO-expressing construct, while the glycosylation mutants were transfected with either just EPO or EPO and UDP-galactose transporter-expressing construct. Lane 1: EPO produced in CHO-K1; lanes 2 and 3: EPO produced in CHO-gmt2; lanes 4 and 5: EPO produced in CHO-gmt3; lanes 6 and 7: EPO produced in CHO-gmt9. Lanes 3, 5, and 7 represent EPO as produced in the respective cell lines when co-transfected with UDP-galactose transporter-expressing construct.

FIG. 2 is a drawing showing FACS analysis of (A) untransfected CHO-K1, (B) untransfected CHO-gmt2, (C) untransfected CHO-gmt3, (D) untransfected CHO-gmt9, (E) GDP-fucose transporter-rescued CHO-gmt3, and (F) GDP-fucose transporter-rescued CHO-gmt9. Transfectants were collected 48 h post-transfection, stained with biotinylated AAL and streptavidin-Cy3, and ran on BD FACSAria III cell sorter.

FIG. 3 is a drawing showing MALDI-TOF mass spectra of N-glycans from purified recombinant EPO-Fc as produced in (A) CHO-K1, (B) CHO-gmt2, (C) CHO-gmt3, and (D) CHO-gmt9. EPO-Fc was transiently produced in each cell line in serum-free medium over 7 days, purified through protein A column, and the released N-glycans were analyzed using MALDI-TOF-MS. Identified N-glycans are as annotated. Blue square, N-acetylglucosamine; Red triangle, fucose; Green circle, mannose; yellow circle, galactose; pink rhombus, sialic acid.

FIG. 4 is a drawing showing MALDI-TOF mass spectra of O-glycans from purified recombinant MUC1-Fc as produced in (A) CHO-K1, (B) CHO-gmt2, (C) CHO-gmt3, and (D) CHO-gmt9. MUC1-Fc was transiently produced in each cell line in serum-free medium over 7 days, purified through protein A column, and the released O-glycans were analyzed using MALDI-TOF-MS. Identified O-glycans are as annotated. Yellow square, N-acetylgalactosamine; Yellow circle, galactose; pink rhombus, sialic acid.

FIG. 5 is a drawing showing MALDI-TOF mass spectra of N-glycans from purified Herceptin as produced in (A) CHO-K1, (B) CHO-gmt2, (C) CHO-gmt3, and (D) CHO-gmt9. Herceptin was transiently produced in each cell line in serum-free medium over 7 days, purified through protein A column, and the released N-glycans were analyzed using MALDI-TOF-MS. Identified N-glycans are as annotated. Blue square, N-acetylglucosamine; Red triangle, fucose; Green circle, mannose; yellow circle, galactose; pink rhombus, sialic acid.

FIG. 6 is a drawing showing HILIC UPLC analysis of the N-glycans from purified Herceptin produced in CHO-K1, CHO-gmt2, CHO-gmt3, and CHO-gmt9. Each glycan peak is annotated according to the presence or absence of the following terminal residues: G for galactose; F for fucose; N for N-acetylglucosamine; Man for mannose; S for sialic acid. G.U., glucose unit.

FIG. 7 is a drawing showing a growth curve of CHO-K1, CHO-gmt2, CHO-gmt3, and CHO-gmt9 grown as suspension cultures in chemically defined serum-free medium. Viable cell density and viability percentage were measured every 24 h until cell viability dropped below 50%.

FIG. 7B shows the structure of the major N-linked oligosaccharides found in human IgG and recombinant IgGs expressed in CHO cells (from Raju (2003), BioProcess International April 2003, 44-53).

FIG. 8 is a diagram showing the design of ZFNs, TALENs and CRISPRs to target the Slc35c1 gene in CHO cells.

FIG. 8A. The binding site sequences for the ZFNs, TALENs and CRISPRs are highlighted in red (underlined). The open reading frame of GDP-fucose transporter in CHO cells is encoded by two exons and the sequence shown here is the first exon of the coding region. The two binding sites for the ZFNs are separated by 6 bps. The two binding sites for the TALENs are separated by 19 bps. Two sites were chosen for CRISPR-Cas9 targeting, namely CRISPR-1 and CRISPR-2.

FIG. 8B. T7E1 mismatch assay to assess the gene modification activities of the designed ZFNs, TALENs and CRISPRs. Genomic DNA of transfected cells was extracted and used as a template for PCR amplification of the region containing the target sites. Purified PCR products were heated and reannealed slowly. They were then digested with the mismatch-sensitive T7 endonuclease 1 (T7E1) that specifically recognizes and cleaves heteroduplexes formed by the hybridization of wild-type and mutant DNA sequences. Asterisks indicate the positions of the T7E1 digestion products.

FIG. 9 is a diagram showing a FACS approach to enrich and isolate GDP-fucose transporter mutant cells generated by ZFNs, TALENs and CRISPRs. CHO-K1 cells were transfected with plasmids encoding the ZFNs, TALENs or CRISPRs. Transfected cells were labelled with biotinylated AAL and Cy3-conjugated streptavidin and the negatively stained cells were isolated by FACS. First round of FACS showed that most of transfected cells were AAL-positive (AAL+ve). The sorting gate for AAL-negative (AAL−ve) cells was set based on unstained CHO cells and the lowest 0.5% of AAL-stained cells were collected and cultured for next round of FACS. Homogeneous population of AAL−ve cells was obtained from each transfected pool after two rounds of FACS. Transfection with human GDP-fucose transporter cDNA rescues the AAL-staining phenotype (+GFT), confirming that these AAL−ve cells lack functional GDP-fucose transporter.

FIG. 10 is a diagram showing MALDI-TOF and HILIC-UPLC profiling of N-glycans on trastuzumab (Herceptin®) and recombinant anti-Her2 antibodies produced by the parental and mutant CHO cells. N-glycans in each antibody preparation were analyzed by MALDI-TOF (A-C) and HILIC-UPLC-QTOF (D-F). For MALDI-TOF experiments, putative glycan structures were assigned by composition matching with theoretical masses of biochemically possible N-glycan structures in CHO cells. For HILIC-UPLC-QTOF experiments, glycan structure assignment was done by library search of GU values, exoglycosidase array fingerprinting, and accurate mass. Only major structures were shown here. Majority of the N-glycans on Herceptin (produced by Roche) (A and D) and our anti-Her2 antibody produced by the parental line (B and E) are fucosylated species such as FA2 (or G0F), FA2G1 isomers (or G1F) and FA2G2 (or G2F). In contrast, no fucosylated N-glycan was detected from mutant-produced anti-Her2 antibody (C and F). Note the consistent glycan profiles under both MALDI-TOF and HILIC-UPLC conditions.

FIG. 11 is a diagram showing the structure elucidation of N-glycans on parental- and mutant-produced anti-Her2 antibody by exoglycosidase digestions. 2-AB-labeled N-glycans were analyzed by HILIC-UPLC-QTOF with or without prior incubation with different exoglycosidases as indicated. Linkage-specific glycan structures were ascertained by following the movement of the corresponding chromatographic peaks as a result of the enzymatic removal of terminal monosaccharides.

FIG. 11A. Such enzymatic fingerprinting analysis revealed the presence of several truncated glycans (M4, A2G1, FM4A1G1) that are closely associated with other more commonly observed structure on HILIC-UPLC.

FIG. 11B. Exoglycosidase array finger printing analysis further confirmed the total absence of fucosylated N-glycans on mutant-produced anti-Her2 antibody. Exoglycosidases used in this experiment are: Arthrobacter ureafaciens sialidase (ABS), bovine kidney α-fucosidase (BKF), bovine testes β-galactosidase (BTG) and Streptococcus pneumoniae β-N-Acetylhexosaminidase (GUH).

FIG. 12 is a diagram showing growth and productivity analysis of GDP-fucose transporter mutants generated by the ZFNs, TALENs and CRISPR-1.

FIG. 12A. Viable cell density and viability of the mutant pools, CHO-HER (ZFNs), CHO-HER (TALENs) and CHO-HER (CRISPR), were compared with parental anti-Her2 antibody-producing CHO-HER cells. Standard deviation (sd) was obtained from the cell count number for triplicate samples. Growth curve was plotted using mean±sd. Dotted lines: Viability. Solid lines: Viable cells/ml.

FIG. 12B. Antibody production by mutant CHO-HER (ZFNs), CHO-HER (TALENs), CHO-HER (CRISPR) and parental CHO-HER were measured using the nephelometric method from day 3 to day 9.

FIG. 12C. Viable cell density and viability of the parental CHO-HER cells and the 6 single clones isolated from the CRISPR-1-generated mutant pool (CRISPR Clone 1 to CRISPR Clone 6).

FIG. 12D. Titers of the antibody produced by the parental CHO-HER cells and the 6 single clones isolated from the CRISPR-generated mutant pool.

FIG. 13 is a diagram showing FACS of CHO cells transfected with CRISPR-2. FACS histogram of cells transfected with CRISPR-2 at first round of sorting indicates a high AAL−ve population of 14.6%. More than 5×10⁴ cells were collected. The collected AAL−ve cells failed to grow and all died. The same results were obtained in repeated experiments.

FIG. 14 is a diagram showing a BLAST search of the guide DNA sequences of CRISPR-1 and CRISPR-2 against the CHO cell genome. The 23 nucleotide sequence GN₂₀GG of CRISPR-1 and CRISPR-2 were subjected to BLAST database search for similar sequences in CHO genome database. Many hits were obtained for both CRISPRs. Unlike CRISPR-1, the search with CRISPR-2 yielded several sequence matches especially at the 3′ end PAM region (red circles). This suggests that CRISPR-2 can potentially bind to many sites other than Slc35c1 and may have off-target effects.

FIG. 15 is a diagram showing MALDI-TOF/MS characterization of N-glycans attached to the EPO-Fc produced by CHO-K1 and CHO-gmt3 cells. N-glycans released from recombinant EPO-Fc produced in CHO-K1 (FIG. 15A) and CHO-gmt3 cells (FIG. 15B) were analyzed by MALDI-TOF. Putative glycan structures were assigned by composition matching with theoretical masses of known N-glycan structures in CHO cells. Note that N-glycans produced by the CHO-gmt3 cells all lack core fucose.

FIG. 16 is a diagram showing FACS approach to enrich and isolate GDP-fucose transporter mutant cells from a pre-existing anti-Her2 antibody-producing CHO DG44 cell line. Suspension culture of the anti-Her2 antibody-producing CHO DG44 cell line (CHO-HER) was transfected with the ZFNs, TALENs or CRISPR-1 targeting the Slc35c1 gene. Transfected cells were labelled with biotinylated AAL and Cy3-conjugated streptavidin for FACS. The AAL−ve cells were collected, cultured and subjected to next round of FACS. After 3 rounds of FACS, homogeneous AAL−ve cell pools were obtained from ZFNs-, TALENs- and CRISPR-1-transfected cells. Transfection of the AAL−ve populations with human GDP-fucose transporter gene rescues the AAL−ve phenotype (+GFT).

FIG. 17 shows an isoelectric focusing gel of human erythropoietin expressed in wild type and mutant cells.

FIG. 18 shows a FACS profile of wild type and mutant cells stained with AAL.

### DETAILED DESCRIPTION

As noted above, the relative proportions of G0, G1, and G2 oligosaccharides in expressed polypeptides vary from batch to batch. Such variations in G0, G1, and G2 glycans affect product quality and bioactivity.

In order to address these problems, we have generated three CHO glycosylation mutants.

One mutant carries a dysfunctional UDP-galactose transporter (Slc35a2) gene (CHO-gmt2). CHO-gmt2 cells produce galactose-free N-glycans and polypeptides, such as antibodies, produced by CHO-gmt2 cells contain mainly G0 N-glycans.

The CHO-gmt2 cell line was deposited on 21 Oct. 2014 at the American Type Culture Collection (ATCC), P.O. Box 1549, Manassas, Va. 20108, United States of America under the Budapest Treaty as accession number PTA-121624.

A second mutant carries a mutated GDP-fucose transporter (Slc35c1) gene (CHO-gmt3). CHO-gmt3 produce fucose-free N-glycans. Polypeptides, such as antibodies, produced by CHO-gmt3 cells have N-glycans lacking core fucose.

In a third mutant, both Slc35a2 and Slc35c1 genes are mutated in the CHO line, CHO-gmt9. CHO-gmt9 therefore has a dysfunctional UDP-galactose transported and a mutated GDP-fucose transporter. Polypeptides, such as antibodies, produced by CHO-gmt9 cells contain mainly G0F0 N-glycans.

The CHO-gmt9 cell line was deposited on 21 Oct. 2014 at the American Type Culture Collection (ATCC), P.O. Box 1549, Manassas, Va. 20108, United States of America under the Budapest Treaty as accession number PTA-121625.

Removal of the core fucose from the N-glycans attached to Asn²⁹⁷ of human IgG1 has been shown to enhance its affinity to its receptor, FcγRIIIa, and thereby improves its antibody-dependent cellular cytotoxicity (ADCC).

We have surprisingly found that cells with these mutations are able to produce much more consistent and homogeneous N-glycans. This results in improvement of batch-to-batch reproducibility. At the same time, we have found that cells with such mutations are able to produce expressed polypeptides such as antibodies with enhanced ADCC.

### Improved Batch to Batch Reproducibility

As described above, the N-glycans attached to the Fc region of recombinant human IgG1 produced by CHO cells can differ dramatically in different stably transfected cells. The differences can also be observed in the antibodies produced by the same cell line but in different batches due to variations in culture conditions.

The N-glycans are mainly of core-fucosylated complex type containing zero or one galactose residue (G0F or G1F), with a small amount of G2F. The relative amount of G0F in total N-glycans in any given sample produced by different stably transfected cells or produced by the same cell line but different batches vary between 40˜70%. This variation represents a serious regulatory concern because of quality assurance (Schiestl et al., 2011; van Berkel et al., 2009).

We therefore provide a method of reducing batch-to-batch variation or increasing homogeneity between batches in the production of a polypeptide such as a recombinantly expressed polypeptide. The method may comprise expressing the polypeptide in a cell such as a Chinese hamster ovary (CHO) cell comprising reduced UDP-galactose transporter activity compared to a wild-type CHO cell.

Optionally, the cell such as a Chinese hamster ovary (CHO) may further comprise reduced GDP-fucose transporter activity compared to a wild-type CHO cell.

The cell may comprise a UDP-galactose transporter (Slc35a2) mutant, as described in further detail below. The cell may optionally comprise a GDP-fucose transporter (Slc35c1) mutant, as described below.

The reduction in batch-to-batch-variation and/or the increase in homogeneity may be as compared to expression in a wild type cell or a cell which has functional UDP-galactose transporter activity.

Expression in a cell which has reduced UDP-galactose transporter activity, such as lacking a functional UDP-galactose transporter (Slc35a2) gene, may result in batch-to-batch variation being reduced by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more, as compared to expression of the same polypeptide in a wild-type cell, or a cell having a functional functional UDP-galactose transporter (Slc35a2) gene.

Expression in a cell which has reduced UDP-galactose transporter activity, such as lacking a functional UDP-galactose transporter (Slc35a2) gene, may result in homogeneity being increased by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more, as compared to expression of the same polypeptide in a wild-type cell, or a cell having a functional UDP-galactose transporter (Slc35a2) gene.

### Improved Antibody-Dependent Cellular Cytotoxicity (ADCC)

Recombinant human IgG1 antibodies have been successfully used as therapeutic drugs to target malignant cells in cancer patients.

Upon binding to the target molecule expressed on cancer cells by the Fab region of the antibody, the Fc region of the antibody binds FcγRIII (CD16) and recruits the effector cells such as natural killer (NK) cells to kill the cancer cell by antibody-dependent cellular cytotoxicity (ADCC).

Studies have shown that the major binding sites of the Fc region that interact with the FcγRIII are located in the hinge region and the CH2 domains of the antibody (Radaev et al., 2001; Sondermann et al., 2000). The affinity between the Fc and the Fc receptor is affected by the structures of the N-glycans attached to the conserved glycosylation site at Asn²⁹⁷ in each of the CH2 domains (Krapp et al., 2003).

It is now widely recognized that removal of the core fucose from the N-glycans attached to Asn²⁹⁷ of human IgG1 significantly enhances its affinity to its receptor, FcγRIIIa, and thereby dramatically improves ADCC (Shields et al., 2002; Shinkawa et al., 2003). Detailed binding analyses indicated that removal of fucose enhanced binding enthalpy and increased binding constant of IgG1 for FcγRIIIa (Okazaki et al., 2004). The structural differences between fucosylated and afucosylated Fc fragments of human IgG1 were compared in X-ray crystallographic and NMR spectroscopic studies. The overall conformations of the fucosylated and afucosylated Fc fragments are similar except for hydration mode around Tyr²⁹⁶. The conformation of Tyr²⁹⁶ is more flexible for FcγRIIIa in afucosylated Fc than in fucosylated Fc (Matsumiya et al., 2007). Tyr²⁹⁶ has already been indicated in the interaction with FcγRIIIa (Radaev et al., 2001; Radaev and Sun, 2002). Enhanced ADCC was not only observed for the afucosylated antibody in in vitro assays, it was also confirmed in vivo in patients or animal models (Cardarelli et al., 2009; Junttila et al., 2010; Niwa et al., 2004; Suzuki et al., 2007).

These data have convincingly shown that removal of fucose from human IgG1 can be a general method for treating cancer patients with antibodies through improved ADCC.

We therefore provide a method of production of a recombinantly expressed polypeptide with enhanced antibody-dependent cellular cytotoxicity (ADCC). The method may comprise expressing the polypeptide in a cell such as a Chinese hamster ovary (CHO) cell comprising reduced GDP-fucose transporter activity compared to a wild-type CHO cell.

Optionally, the cell such as a Chinese hamster ovary (CHO) may further comprise reduced UDP-galactose transporter compared to a wild-type CHO cell.

The cell may comprise a GDP-fucose transporter (Slc35c1) mutant, as described in further detail below. The cell may optionally comprise a UDP-galactose transporter (Slc35a2) mutant, as described below.

The enhanced antibody-dependent cellular cytotoxicity (ADCC) may be as compared to expression in a wild type cell or a cell which has functional GDP-fucose transporter activity.

We provide for the use of a such a cell in a method of expression of a polypeptide such as a recombinant polypeptide. Where the polypeptide comprises an IgG1, the expressed polypeptide may display improved, increased or enhanced affinity for the receptor FcγRIIIa when compared to a polypeptide expressed from a wild-type CHO cell. The polypeptide may display enhanced Antibody-Dependent Cellular Cytotoxicity (ADCC), when compared to a polypeptide expressed from a wild-type CHO cell.

Expression in a cell which has reduced GDP-fucose transporter activity, such as lacking a functional GDP-fucose transporter (Slc35c1) gene, may result in ADCC being increased by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more, as compared to expression of the same polypeptide in a wild-type cell, or a cell having a functional GDP-fucose transporter (Slc35c1) gene.

Expression in a cell which has reduced GDP-fucose transporter activity, such as lacking a functional GDP-fucose transporter (Slc35c1) gene, may result in ADCC being increased by 100% or more, 150% or more, 200% or more, 250% or more, 300% or more, 350% or more, 400% or more, 450% or more, 500% or more, 550% or more, 600% or more, 650% or more, 700% or more, 750% or more, 800% or more, 850% or more, 900% or more or 950% or more, as compared to expression of the same polypeptide in a wild-type cell, or a cell having a functional GDP-fucose transporter (Slc35c1) gene.

### Improved ADCC and Batch-to-Batch Reproducibility

We further provide a method of production of a recombinantly expressed polypeptide with higher antibody-dependent cellular cytotoxicity (ADCC) with reduced batch-to-batch variation or increased homogeneity between batches.

The method may comprise expressing the polypeptide in a cell such as a Chinese hamster ovary (CHO) cell comprising reduced UDP-galactose transporter activity and reduced GDP-fucose transporter activity compared to a wild-type CHO cell.

The cell may comprise a UDP-galactose transporter (Slc35a2) mutant and a GDP-fucose transporter (Slc35c1) mutant, as described below. The cell may comprise a CHO-gmt9 cell, deposited on 21 Oct. 2014 at the American Type Culture Collection (ATCC), P.O. Box 1549, Manassas, Va. 20108, United States of America under the Budapest Treaty as accession number PTA-121625.

The reduction in batch-to-batch-variation and/or the increase in homogeneity and/or the enhancement in ADCC may be as compared to expression in a wild type cell or a cell which has functional UDP-galactose transporter activity and functional GDP-fucose transporter activity.

Expression in a such a cell may result in batch-to-batch variation being reduced by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more, as compared to expression of the same polypeptide in a wild-type cell.

Expression in such a cell may result in homogeneity being increased by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more, as compared to expression of the same polypeptide in a wild-type cell.

Expression in such a cell may result in ADCC being increased by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more, as compared to expression of the same polypeptide in a wild-type cell.

Expression such a cell may result in ADCC being increased by 100% or more, 150% or more, 200% or more, 250% or more, 300% or more, 350% or more, 400% or more, 450% or more, 500% or more, 550% or more, 600% or more, 650% or more, 700% or more, 750% or more, 800% or more, 850% or more, 900% or more or 950% or more, as compared to expression of the same polypeptide in a wild-type cell.

### Changes in Expression Profile

Increasing G0F N-Glycans

The N-glycans produced by a cell as described in this document, such as a cell lacking functional UDP-galactose transporter, may comprise G0F. The N-glycans may comprise lowered G1F or G2F N-glycans. The N-glycans may comprise no significant amount of G1F or G2F N-glycans.

The N-glycans produced by a cell as described in this document, such as a cell lacking functional UDP-galactose transporter, may show an increase in G0F N-glycans.

For example, the N-glycans produced by a cell as described in this document, such as a cell lacking functional UDP-galactose transporter, may comprise primarily G0F.

We therefore provide a method of increasing G0F N-glycans in a expressed polypeptide such as a recombinant polypeptide. N-glycans of the G0F type may be increased in number or proportion, with respect to the total N-glycans in or on the expressed polypeptide.

The method may comprise expressing the polypeptide in a cell such as a Chinese hamster ovary (CHO) cell comprising reduced UDP-galactose transporter activity compared to a wild-type CHO cell.

The N-glycans may comprise 50% or more G0F N-glycans, 55% or more G0F N-glycans, 60% or more G0F N-glycans, 65% or more G0F N-glycans, 70% or more G0F N-glycans, 75% or more G0F N-glycans, 80% or more G0F N-glycans, 85% or more G0F N-glycans or 90% or more G0F N-glycans.

The N-glycans may comprise 91% or more G0F N-glycans, 92% or more G0F N-glycans, 93% or more G0F N-glycans, 94% or more G0F N-glycans, 95% or more G0F N-glycans, 96% or more G0F N-glycans, 97% or more G0F N-glycans, 98% or more G0F N-glycans or 99% or more G0F N-glycans.

The N-glycans may comprise 99.91% or more G0F N-glycans, 99.92% or more G0F N-glycans, 99.93% or more G0F N-glycans, 99.4% or more G0F N-glycans, 99.5% or more G0F N-glycans, 99.6% or more G0F N-glycans, 99.7% or more G0F N-glycans, 99.8% or more G0F N-glycans or 99.9% or more G0F N-glycans.

The N-glycans may comprise 99.91% or more G0F N-glycans, 99.92% or more G0F N-glycans, 99.93% or more G0F N-glycans, 99.94% or more G0F N-glycans, 99.95% or more G0F N-glycans, 99.96% or more G0F N-glycans, 99.97% or more G0F N-glycans, 99.98% or more G0F N-glycans or 99.99% or more G0F N-glycans.

The N-glycans may comprise 99.991% or more G0F N-glycans, 99.992% or more G0F N-glycans, 99.993% or more G0F N-glycans, 99.994% or more G0F N-glycans, 99.995% or more G0F N-glycans, 99.996% or more G0F N-glycans, 99.997% or more G0F N-glycans, 99.998% or more G0F N-glycans or 99.999% or more G0F N-glycans.

The N-glycans may comprise 99.9991% or more G0F N-glycans, 99.9992% or more G0F N-glycans, 99.9993% or more G0F N-glycans, 99.9994% or more G0F N-glycans, 99.9995% or more G0F N-glycans, 99.9996% or more G0F N-glycans, 99.9997% or more G0F N-glycans, 99.9998% or more G0F N-glycans or 99.9999% or more G0F N-glycans.

Decreasing G1F N-Glycans

The N-glycans produced by a cell as described in this document, such as a cell lacking functional UDP-galactose transporter, may show a decrease in G1F N-glycans.

We therefore provide a method of decreasing G1F N-glycans in a expressed polypeptide such as a recombinant polypeptide. N-glycans of the G1F type may be decreased in number or proportion, with respect to the total N-glycans in or on the expressed polypeptide.

The method may comprise expressing the polypeptide in a cell such as a Chinese hamster ovary (CHO) cell comprising reduced UDP-galactose transporter activity compared to a wild-type CHO cell.

The N-glycans may comprise 50% or less G1F N-glycans, 45% or less G1F N-glycans, 40% or less G1F N-glycans, 35% or less G1F N-glycans, 30% or less G1F N-glycans, 25% or less G1F N-glycans, 20% or less G1F N-glycans, 15% or less G1F N-glycans or 10% or less G1F N-glycans.

The N-glycans may comprise 9% or less G1F N-glycans, 8% or less G1F N-glycans, 7% or less G1F N-glycans, 6% or less G1F N-glycans, 5% or less G1F N-glycans, 4% or less G1F N-glycans, 3% or less G1F N-glycans, 2% or less G1F N-glycans or 1% or less G1F N-glycans.

The N-glycans may comprise 0.9% or less G1F N-glycans, 0.8% or less G1F N-glycans, 0.7% or less G1F N-glycans, 0.6% or less G1F N-glycans, 0.5% or less G1F N-glycans, 0.4% or less G1F N-glycans, 0.3% or less G1F N-glycans, 0.2% or less G1F N-glycans or 0.1% or less G1F N-glycans.

The N-glycans may comprise 0.09% or less G1F N-glycans, 0.08% or less G1F N-glycans, 0.07% or less G1F N-glycans, 0.06% or less G1F N-glycans, 0.05% or less G1F N-glycans, 0.04% or less G1F N-glycans, 0.03% or less G1F N-glycans, 0.02% or less G1F N-glycans or 0.01% or less G1F N-glycans.

The N-glycans may comprise 0.009% or less G1F N-glycans, 0.008% or less G1F N-glycans, 0.007% or less G1F N-glycans, 0.006% or less G1F N-glycans, 0.005% or less G1F N-glycans, 0.004% or less G1F N-glycans, 0.003% or less G1F N-glycans, 0.002% or less G1F N-glycans or 0.001% or less G1F N-glycans.

Decreasing G2F N-Glycans

The N-glycans produced by a cell as described in this document, such as a cell lacking functional UDP-galactose transporter, may show a decrease in G2F N-glycans.

We therefore provide a method of decreasing G2F N-glycans in a expressed polypeptide such as a recombinant polypeptide. N-glycans of the G2F type may be decreased in number or proportion, with respect to the total N-glycans in or on the expressed polypeptide.

The method may comprise expressing the polypeptide in a cell such as a Chinese hamster ovary (CHO) cell comprising reduced UDP-galactose transporter activity compared to a wild-type CHO cell.

The N-glycans may comprise 50% or less G2F N-glycans, 45% or less G2F N-glycans, 40% or less G2F N-glycans, 35% or less G2F N-glycans, 30% or less G2F N-glycans, 25% or less G2F N-glycans, 20% or less G2F N-glycans, 15% or less G2F N-glycans or 10% or less G2F N-glycans.

The N-glycans may comprise 9% or less G2F N-glycans, 8% or less G2F N-glycans, 7% or less G2F N-glycans, 6% or less G2F N-glycans, 5% or less G2F N-glycans, 4% or less G2F N-glycans, 3% or less G2F N-glycans, 2% or less G2F N-glycans or 1% or less G2F N-glycans.

The N-glycans may comprise 9% or less G2F N-glycans, 8% or less G2F N-glycans, 7% or less G2F N-glycans, 6% or less G2F N-glycans, 5% or less G2F N-glycans, 4% or less G2F N-glycans, 3% or less G2F N-glycans, 2% or less G2F N-glycans or 1% or less G2F N-glycans.

The N-glycans may comprise 0.9% or less G2F N-glycans, 0.8% or less G2F N-glycans, 0.7% or less G2F N-glycans, 0.6% or less G2F N-glycans, 0.5% or less G2F N-glycans, 0.4% or less G2F N-glycans, 0.3% or less G2F N-glycans, 0.2% or less G2F N-glycans or 0.1% or less G2F N-glycans.

The N-glycans may comprise 0.09% or less G2F N-glycans, 0.08% or less G2F N-glycans, 0.07% or less G2F N-glycans, 0.06% or less G2F N-glycans, 0.05% or less G2F N-glycans, 0.04% or less G2F N-glycans, 0.03% or less G2F N-glycans, 0.02% or less G2F N-glycans or 0.01% or less G2F N-glycans.

The N-glycans may comprise 0.009% or less G2F N-glycans, 0.008% or less G2F N-glycans, 0.007% or less G2F N-glycans, 0.006% or less G2F N-glycans, 0.005% or less G2F N-glycans, 0.004% or less G2F N-glycans, 0.003% or less G2F N-glycans, 0.002% or less G2F N-glycans or 0.001% or less G2F N-glycans.

Increasing Fucose Free N-Glycans

The N-glycans produced by a cell as described in this document, such as a cell lacking GDP-fucose transporter (Slc35c1) gene, may comprise fucose-free N-glycans.

An example of such a cell is the CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

The N-glycans produced by a cell as described in this document, such as a cell lacking functional GDP-fucose transporter, may show an increase in G0F0 N-glycans.

For example, they may comprise mainly G0F0 complex type N-glycans.

We therefore provide a method of increasing G0F0 N-glycans in a expressed polypeptide such as a recombinant polypeptide. N-glycans of the G0F0 type may be increased in number or proportion, with respect to the total N-glycans in or on the expressed polypeptide.

The method may comprise expressing the polypeptide in a cell such as a Chinese hamster ovary (CHO) cell comprising reduced GDP-fucose transporter activity compared to a wild-type CHO cell.

The N-glycans may comprise 50% or more G0F0 N-glycans, 55% or more G0F0 N-glycans, 60% or more G0F0 N-glycans, 65% or more G0F0 N-glycans, 70% or more G0F0 N-glycans, 75% or more G0F0 N-glycans, 80% or more G0F0 N-glycans, 85% or more G0F0 N-glycans or 90% or more G0F0 N-glycans.

The N-glycans may comprise 91% or more G0F0 N-glycans, 92% or more G0F0 N-glycans, 93% or more G0F0 N-glycans, 94% or more G0F0 N-glycans, 95% or more G0F0 N-glycans, 96% or more G0F0 N-glycans, 97% or more G0F0 N-glycans, 98% or more G0F0 N-glycans or 99% or more G0F0 N-glycans.

The N-glycans may comprise 99.1% or more G0F0 N-glycans, 99.2% or more G0F0 N-glycans, 99.3% or more G0F0 N-glycans, 99.4% or more G0F0 N-glycans, 99.5% or more G0F0 N-glycans, 99.6% or more G0F0 N-glycans, 99.7% or more G0F0 N-glycans, 99.8% or more G0F0 N-glycans or 99.9% or more G0F0 N-glycans.

The N-glycans may comprise 99.91% or more G0F0 N-glycans, 99.92% or more G0F0 N-glycans, 99.93% or more G0F0 N-glycans, 99.94% or more G0F0 N-glycans, 99.95% or more G0F0 N-glycans, 99.96% or more G0F0 N-glycans, 99.97% or more G0F0 N-glycans, 99.98% or more G0F0 N-glycans or 99.99% or more G0F0 N-glycans.

The N-glycans may comprise 99.991% or more G0F0 N-glycans, 99.992% or more G0F0 N-glycans, 99.993% or more G0F0 N-glycans, 99.994% or more G0F0 N-glycans, 99.995% or more G0F0 N-glycans, 99.996% or more G0F0 N-glycans, 99.997% or more G0F0 N-glycans, 99.998% or more G0F0 N-glycans or 99.999% or more G0F0 N-glycans.

The N-glycans may comprise 99.9991% or more G0F0 N-glycans, 99.9992% or more G0F0 N-glycans, 99.9993% or more G0F0 N-glycans, 99.9994% or more G0F0 N-glycans, 99.9995% or more G0F0 N-glycans, 99.9996% or more G0F0 N-glycans, 99.9997% or more G0F0 N-glycans, 99.9998% or more G0F0 N-glycans or 99.9999% or more G0F0 N-glycans.

Increasing G0 N-Glycans

The N-glycans produced by a cell as described in this document, such as a cell lacking GDP-fucose transporter (Slc35c1) gene, may comprise fucose-free N-glycans.

An example of such a cell is the CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

The N-glycans produced by a cell as described in this document, such as a cell lacking functional GDP-fucose transporter, may show an increase in G0 N-glycans.

For example, they may comprise mainly G0 complex type N-glycans.

We therefore provide a method of increasing G0 N-glycans in a expressed polypeptide such as a recombinant polypeptide. N-glycans of the G0 type may be increased in number or proportion, with respect to the total N-glycans in or on the expressed polypeptide.

The method may comprise expressing the polypeptide in a cell such as a Chinese hamster ovary (CHO) cell comprising reduced GDP-fucose transporter activity compared to a wild-type CHO cell.

The N-glycans may comprise 50% or more G0 N-glycans, 55% or more G0 N-glycans, 60% or more G0 N-glycans, 65% or more G0 N-glycans, 70% or more G0 N-glycans, 75% or more G0 N-glycans, 80% or more G0 N-glycans, 85% or more G0 N-glycans or 90% or more G0 N-glycans.

The N-glycans may comprise 91% or more G0 N-glycans, 92% or more G0 N-glycans, 93% or more G0 N-glycans, 94% or more G0 N-glycans, 95% or more G0 N-glycans, 96% or more G0 N-glycans, 97% or more G0 N-glycans, 98% or more G0 N-glycans or 99% or more G0 N-glycans.

The N-glycans may comprise 99.1% or more G0 N-glycans, 99.2% or more G0 N-glycans, 99.3% or more G0 N-glycans, 99.4% or more G0 N-glycans, 99.5% or more G0 N-glycans, 99.6% or more G0 N-glycans, 99.7% or more G0 N-glycans, 99.8% or more G0 N-glycans or 99.9% or more G0 N-glycans.

The N-glycans may comprise 99.91% or more G0 N-glycans, 99.92% or more G0 N-glycans, 99.93% or more G0 N-glycans, 99.94% or more G0 N-glycans, 99.95% or more G0 N-glycans, 99.96% or more G0 N-glycans, 99.97% or more G0 N-glycans, 99.98% or more G0 N-glycans or 99.99% or more G0 N-glycans.

The N-glycans may comprise 99.991% or more G0 N-glycans, 99.992% or more G0 N-glycans, 99.993% or more G0 N-glycans, 99.994% or more G0 N-glycans, 99.995% or more G0 N-glycans, 99.996% or more G0 N-glycans, 99.997% or more G0 N-glycans, 99.998% or more G0 N-glycans or 99.999% or more G0 N-glycans.

The N-glycans may comprise 99.9991% or more G0 N-glycans, 99.9992% or more G0 N-glycans, 99.9993% or more G0 N-glycans, 99.9994% or more G0 N-glycans, 99.9995% or more G0 N-glycans, 99.9996% or more G0 N-glycans, 99.9997% or more G0 N-glycans, 99.9998% or more G0 N-glycans or 99.9999% or more G0 N-glycans.

Decreasing G1 N-Glycans

The N-glycans produced by a cell lacking functional GDP-fucose transporter may show a decrease in G1 N-glycans.

We therefore provide a method of decreasing G1 N-glycans in a expressed polypeptide such as a recombinant polypeptide. N-glycans of the G1 type may be decreased in number or proportion, with respect to the total N-glycans in or on the expressed polypeptide.

The method may comprise expressing the polypeptide in a cell such as a Chinese hamster ovary (CHO) cell comprising reduced GDP-fucose transporter activity compared to a wild-type CHO cell.

The N-glycans may comprise 50% or less G1 N-glycans, 45% or less G1 N-glycans, 40% or less G1 N-glycans, 35% or less G1 N-glycans, 30% or less G1 N-glycans, 25% or less G1 N-glycans, 20% or less G1 N-glycans, 15% or less G1 N-glycans or 10% or less G1 N-glycans.

The N-glycans may comprise 9% or less G1 N-glycans, 8% or less G1 N-glycans, 7% or less G1 N-glycans, 6% or less G1 N-glycans, 5% or less G1 N-glycans, 4% or less G1 N-glycans, 3% or less G1 N-glycans, 2% or less G1 N-glycans or 1% or less G1 N-glycans.

The N-glycans may comprise 0.9% or less G1 N-glycans, 0.8% or less G1 N-glycans, 0.7% or less G1 N-glycans, 0.6% or less G1 N-glycans, 0.5% or less G1 N-glycans, 0.4% or less G1 N-glycans, 0.3% or less G1 N-glycans, 0.2% or less G1 N-glycans or 0.1% or less G1 N-glycans.

The N-glycans may comprise 0.09% or less G1 N-glycans, 0.08% or less G1 N-glycans, 0.07% or less G1 N-glycans, 0.06% or less G1 N-glycans, 0.05% or less G1 N-glycans, 0.04% or less G1 N-glycans, 0.03% or less G1 N-glycans, 0.02% or less G1 N-glycans or 0.01% or less G1 N-glycans.

The N-glycans may comprise 0.009% or less G1 N-glycans, 0.008% or less G1 N-glycans, 0.007% or less G1 N-glycans, 0.006% or less G1 N-glycans, 0.005% or less G1 N-glycans, 0.004% or less G1 N-glycans, 0.003% or less G1 N-glycans, 0.002% or less G1 N-glycans or 0.001% or less G1 N-glycans.

Decreasing G2 N-Glycans

The N-glycans produced by a cell lacking functional GDP-fucose transporter may show a decrease in G2 N-glycans.

We therefore provide a method of decreasing G2 N-glycans in a expressed polypeptide such as a recombinant polypeptide. N-glycans of the G2 type may be decreased in number or proportion, with respect to the total N-glycans in or on the expressed polypeptide.

The method may comprise expressing the polypeptide in a cell such as a Chinese hamster ovary (CHO) cell comprising reduced GDP-fucose transporter activity compared to a wild-type CHO cell.

The N-glycans may comprise 50% or less G2 N-glycans, 45% or less G2 N-glycans, 40% or less G2 N-glycans, 35% or less G2 N-glycans, 30% or less G2 N-glycans, 25% or less G2 N-glycans, 20% or less G2 N-glycans, 15% or less G2 N-glycans or 10% or less G2 N-glycans.

The N-glycans may comprise 9% or less G2 N-glycans, 8% or less G2 N-glycans, 8% or less G2 N-glycans, 6% or less G2 N-glycans, 5% or less G2 N-glycans, 4% or less G2 N-glycans, 3% or less G2 N-glycans, 2% or less G2 N-glycans or 1% or less G2 N-glycans.

The N-glycans may comprise 0.9% or less G2 N-glycans, 0.8% or less G2 N-glycans, 0.7% or less G2 N-glycans, 0.6% or less G2 N-glycans, 0.5% or less G2 N-glycans, 0.4% or less G2 N-glycans, 0.3% or less G2 N-glycans, 0.2% or less G2 N-glycans or 0.1% or less G2 N-glycans.

The N-glycans may comprise 0.09% or less G2 N-glycans, 0.08% or less G2 N-glycans, 0.07% or less G2 N-glycans, 0.06% or less G2 N-glycans, 0.05% or less G2 N-glycans, 0.04% or less G2 N-glycans, 0.03% or less G2 N-glycans, 0.02% or less G2 N-glycans or 0.01% or less G2 N-glycans.

The N-glycans may comprise 0.009% or less G2 N-glycans, 0.008% or less G2 N-glycans, 0.007% or less G2 N-glycans, 0.006% or less G2 N-glycans, 0.005% or less G2 N-glycans, 0.004% or less G2 N-glycans, 0.003% or less G2 N-glycans, 0.002% or less G2 N-glycans or 0.001% or less G2 N-glycans.

### Assay for Protein-Derived N-Linked Oligosaccharides

The monosaccharide composition of purified expressed protein may be characterized by modified high-performance anion exchange chromatography. The following protocol is an example and is not meant to be limiting.

Monosaccharides are released from an aliquot of protein by heating with 4 M trifluoroacetic acid at 100 8 C for 2 h and dried under a vacuum.

The monosaccharides reconstituted in sterile distilled water are analyzed using a waveform and DX500 system (DIONEX, Sunnyvale, Calif.). A CarboPac PA-1 column (DIONEX) is used to resolve monosaccharides in 18 mM sodium hydroxide solution with a flow rate of 0.8 mL/min at 35° C. as described previously (Shinkawa et al. 2003).

The oligosaccharide profile of each purified protein is characterized by modified matrix-assisted laser desorption/ionization time-of-flight mass spectrometry (MALDI-TOF MS) with a positive ion mode as described previously (Papac et al. 1998); N-linked oligosaccharides are released from 30 mg of IgG1 by incubation with lunit of recombinant peptide-N-glycosidase F (PNGaseF; Sigma-Aldrich) for 18 h at 378 C in 10 mM Tris-acetate (pH 8.3).

The released oligo-saccharides are recovered after precipitation of the protein with 75% ethanol. Following drying of the recovered supernatant, the oligosaccharides are dissolved in 13 mM acetic acid and incubated at room temperature for 2 h. The acid-treated samples are desalted with cation-exchange resin (AG50W-X8, hydrogen form; BioRad, Hercules, Calif.) and dried in a vacuum.

The dried samples are dissolved in deionized water and mixed with the matrix super-DHB solution (Bruker Daltonics) to be characterized by a MALDI-TOF MS spectrometer Reflex III (Bruker Daltonik GmbH, Bremen, Fahrenheitstr, Germany) equipped with delayed extraction.

All samples are irradiated with ultraviolet light (337 nm) from an N2 laser; positive ions are accelerated to 20 kV and analyzed in a reflectron mode. The oligosaccharide standards (TAKARA BIO Inc., Shiga, Japan) are employed.

### UDP-Galactose Transporter (Slc35a2) Sequence

The Chinese hamster UDP-Galactose Transporter gene (Slc35a2, GenBank: NM_001244019.1), also referred to as the Cricetulus griseus solute carrier family 35 (UDP-galactose transporter), member A2 (Slc35a2), mRNA sequence, has the following nucleotide sequence:

NCBI Reference Sequence: NM_001244019.1

>gi|345842342: 10-1206 Cricetulus griseus solute
carrier family 35 (UDP-galactose transporter),
member A2 (Slc35a2), mRNA
ATGGCAGCGGTTGGGGTTGGCGGATCTGCCGCGGCGGCCGGGCCAGGGG
CCGTATCCGCTGGCGCGCTGGAGCCTGGGTCCGCTACAGCGGCTCACCG
GCGCCTCAAGTACATATCCTTAGCTGTGCTGGTGGTCCAGAACGCCTCC
CTCATCCTTAGCATCCGATATGCTCGTACACTGCCTGGTGATCGCTTCT
TTGCCACCACCGCTGTGGTCATGGCTGAAGTGCTTAAAGGTGTCACCTG
TCTCCTGCTGCTCTTCGCCCAGAAGAGGGGTAATGTGAAGCACCTGGTT
CTCTTCCTCCACGAGGCTGTCCTGGTGCAATATGTGGACACACTCAAGC
TCGCGGTGCCCTCTCTCATCTATACTTTGCAGAATAACCTCCAGTATGT
TGCCATCTCCAACCTGCCAGCTGCCACTTTCCAGGTGACATATCAGCTC
AAGATCCTGACTACAGCACTGTTCTCCGTGCTCATGTTGAACCGCAGCC
TCTCACGCCTGCAGTGGGCCTCCCTGCTGCTGCTCTTCACTGGTGTGGC
GATTGTCCAGGCACAGCAAGCTGGTGGGGGTGGCCCACGGCCACTAGAT
CAGAATCCCGGGGCCGGACTAGCAGCTGTGGTGGCCTCCTGTCTCTCCT
CAGGCTTTGCAGGGGTATACTTTGAGAAGATCCTCAAAGGCAGCTCAGG
TTCTGTGTGGCTGCGTAACCTCCAACTAGGCCTCTTTGGCACAGCACTG
GGCCTGGTGGGGCTCTGGTGGGCTGAAGGCACTGCTGTGGCCCGTCGAG
GCTTCTTCTTTGGATACACGCCTGCTGTCTGGGGGGTGGTACTAAACCA
AGCCTTTGGTGGGCTACTGGTGGCTGTTGTAGTCAAGTACGCTGACAAC
ATCCTCAAGGGCTTTGCCACCTCCCTGTCTATTGTGCTGTCCACTGTTG
CCTCCATTCGCCTCTTTGGCTTTCACCTGGACCCATTATTTGCCCTGGG
CGCTGGGCTCGTCATTGGTGCCGTCTACCTCTACAGCCTTCCCCGAAGT
GCAGTCAAAGCCATAACTTCTGCCTCTGCCTCTGCCTCTGCCTCTGGGC
CCTGCATTCACCAGCAGCCTCCTGGGCAGCCACCACCACCACCGCAGCT
GTCTTCTCGAGGAGACCTCATCACGGAGCCCTTTCTGCCAAAGTTGCTC
ACCAAGGTGAAGGGTTCGTAG

The polypeptide sequence of the Chinese hamster UDP-Galactose Transporter is as follows:

UDP-galactose translocator protein sequence
[Cricetulus griseus] NCBI Reference Sequence:
NP_001230948.1 >gi|345842343|ref|NP_001230948.1|
UDP-galactose translocator [Cricetulus griseus]
MAAVGVGGSAAAAGPGAVSAGALEPGSATAAHRRLKYISLAVLVVQNAS
LILSIRYARTLPGDRFFATTAVVMAEVLKGVTCLLLLFAQKRGNVKHLV
LFLHEAVLVQYVDTLKLAVPSLIYTLQNNLQYVAISNLPAATFQVTYQL
KILTTALFSVLMLNRSLSRLQWASLLLLFTGVAIVQAQQAGGGGPRPLD
QNPGAGLAAVVASCLSSGFAGVYFEKILKGSSGSVWLRNLQLGLFGTAL
GLVGLWWAEGTAVARRGFFFGYTPAVWGVVLNQAFGGLLVAVVVKYADN
ILKGFATSLSIVLSTVASIRLFGFHLDPLFALGAGLVIGAVYLYSLPRS
AVKAITSASASASASGPCIHQQPPGQPPPPPQLSSRGDLITEPFLPKLL
TKVKGS

We further provide for mutant UDP-Galactose Transporter polypeptides, as well as fragments, variants, derivatives and homologoues thereof.

The mutant UDP-Galactose Transporter polypeptide may comprise a sequence shown below or a variant, homologue, derivative or fragment thereof.

### GDP-Fucose Transporter (Slc35c1) Sequence

The Chinese hamster GDP-Fucose Transporter gene (Slc35C1, GenBank: 001246808.1), also known as the Chinese hamster [Cricetulus griseus] solute carrier family 35 (GDP-fucose transporter), member C1 (Slc35c1), mRNA sequence has the following nucleotide sequence:

NCBI Reference Sequence:

NM_001246808.1>gi|350538844|ref|
NM_001246808.1|Cricetulus griseus solute
carrier family 35 (GDP-fucose transporter),
member C1 (Slc35c1), mRNA
ATGAACAGGGCGCCTCTGAAGCGGTCCAGGATCCTGCGCATGGCGCTGA
CTGGAGGCTCCACTGCCTCTGAGGAGGCAGATGAGGACAGCAGGAACAA
GCCGTTTCTGCTGCGGGCGCTGCAGATCGCGCTGGTCGTCTCTCTCTAC
TGGGTCACCTCCATCTCCATGGTATTCCTCAACAAGTACCTGCTGGACA
GCCCCTCCCTGCAGCTGGATACCCCTATCTTCGTCACTTTCTACCAATG
CCTGGTGACCTCTCTGCTGTGCAAGGGCCTCAGCACTCTGGCCACCTGC
TGCCCTGGCACCGTTGACTTCCCCACCCTGAACCTGGACCTTAAGGTGG
CCCGCAGCGTGCTGCCACTGTCGGTAGTCTTCATTGGCATGATAAGTTT
CAATAACCTCTGCCTCAAGTACGTAGGGGTGGCCTTCTACAACGTGGGG
CGCTCGCTCACCACCGTGTTCAATGTGCTTCTGTCCTACCTGCTGCTCA
AACAGACCACTTCCTTCTATGCCCTGCTCACATGTGGCATCATCATTGG
TGGTTTCTGGCTGGGTATAGACCAAGAGGGAGCTGAGGGCACCCTGTCC
CTCATAGGCACCATCTTCGGGGTGCTGGCCAGCCTCTGCGTCTCCCTCA
ATGCCATCTATACCAAGAAGGTGCTCCCAGCAGTGGACAACAGCATCTG
GCGCCTAACCTTCTATAACAATGTCAATGCCTGTGTGCTCTTCTTGCCC
CTGATGGTTCTGCTGGGTGAGCTCCGTGCCCTCCTTGACTTTGCTCATC
TGTACAGTGCCCACTTCTGGCTCATGATGACGCTGGGTGGCCTCTTCGG
CTTTGCCATTGGCTATGTGACAGGACTGCAGATCAAATTCACCAGTCCC
CTGACCCACAATGTATCAGGCACAGCCAAGGCCTGTGCGCAGACAGTGC
TGGCCGTGCTCTACTATGAAGAGACTAAGAGCTTCCTGTGGTGGACAAG
CAACCTGATGGTGCTGGGTGGCTCCTCAGCCTATACCTGGGTCAGGGGC
TGGGAGATGCAGAAGACCCAAGAGGACCCCAGCTCCAAAGAGGGTGAGA
AGAGTGCTATCAGGGTGTGA

The polypeptide sequence of the Chinese hamster [Cricetulus griseus] GDP-fucose transporter 1 (SLC35C1) protein is as follows:

NCBI Reference Sequence: NP_001233737.1>gi|
350538845|ref|NP_001233737.1|
GDP-fucose transporter 1 [Cricetulus griseus]
MNRAPLKRSRILRMALTGGSTASEEADEDSRNKPFLLRALQIALVVSLY
WVTSISMVFLNKYLLDSPSLQLDTPIFVTFYQCLVTSLLCKGLSTLATC
CPGTVDFPTLNLDLKVARSVLPLSVVFIGMISFNNLCLKYVGVAFYNVG
RSLTTVFNVLLSYLLLKQTTSFYALLTCGIIIGGFWLGIDQEGAEGTLS
LIGTIFGVLASLCVSLNAIYTKKVLPAVDNSIWRLTFYNNVNACVLFLP
LMVLLGELRALLDFAHLYSAHFWLMMTLGGLFGFAIGYVTGLQIKFTSP
LTHNVSGTAKACAQTVLAVLYYEETKSFLWWTSNLMVLGGSSAYTWVRG
WEMQKTQEDPSSKEGEKSAIRV

The mutant GDP-fucose transporter polypeptide may comprise a sequence shown above or a variant, homologue, derivative or fragment thereof.

### UDP-Galactose Transporter (Slc35a2) Mutants

We describe a UDP-galactose transporter (Slc35a2) mutant. The mutant may comprise a UDP-galactose transporter (Slc35a2) polynucleotide or polypeptide comprising a mutation in its sequence. The mutation may comprise a loss of function mutation.

The mutant may comprise a mutant UDP-galactose transport polypeptide as described below. The mutant may comprise a mutant UDP-galactose transport nucleic acid as described below.

The mutant UDP-galactose transport polypeptide or mutant UDP-galactose transport nucleic acid may comprise a polypeptide or a nucleotide sequence (as the case may be), as set out in the sections labelled “Mutant UDP-Galactose Transporter—CHO-gmt2”, “Mutant UDP-Galactose Transporter Sequence—CHO-gmt9 (Clone GalF7)”, “Mutant UDP-Galactose Transporter Sequence—CHO-gmt9 (Clone GalF102)”, “Mutant UDP-Galactose Transporter Sequence—CHO-gmt9 (Clone GalF103)” or “Mutant UDP-Galactose Transporter Sequence—CHO-gmt9 (Clone GalF109)” below.

We further provide for cells such as CHO cells comprising such a UDP-galactose transporter (Slc35a2) mutant and their use in expression of proteins such as recombinant polypeptides.

### GDP-Fucose Transporter (Slc35c1) Mutants

We describe a GDP-fucose transporter (Slc35c1) mutant. The mutant may comprise a GDP-fucose transporter (Slc35c1) polynucleotide or polypeptide comprising a mutation in its sequence. The mutation may comprise a loss of function mutation.

The mutant may comprise a mutant GDP-fucose transport polypeptide as described below. The mutant may comprise a mutant GDP-fucose transport nucleic acid as described below.

The mutant GDP-fucose transport polypeptide or mutant GDP-fucose transport nucleic acid may comprise a polypeptide or a nucleotide sequence (as the case may be), as set out in the sections labelled “Mutant GDP-Fucose Transporter Sequence—CHO-gmt3 clone #2 Allele 1”, “Mutant GDP-Fucose Transporter Sequence—CHO-gmt3 clone #2 Allele 2”, “Mutant GDP-Fucose Transporter Sequence—CHO-gmt3 clone #3 Allele 1”, “Mutant GDP-Fucose Transporter Sequence—CHO-gmt3 clone #3 Allele 2”, “Mutant GDP-Fucose Transporter Sequence—CHO-gmt3 clone #4 Allele 1”, “Mutant GDP-Fucose Transporter Sequence—CHO-gmt3 clone #4 Allele 2”, “Mutant GDP-Fucose Transporter Sequence—CHO-gmt3 clone #6 Allele 1”, “Mutant GDP-Fucose Transporter Sequence—CHO-gmt3 clone #8 Allele 1”, “Mutant GDP-Fucose Transporter Sequence—CHO-gmt3 clone #8 Allele 2”, “Mutant GDP-Fucose Transporter Sequence—CHO-gmt3 clone #9 Allele 1”, “Mutant GDP-Fucose Transporter Sequence—CHO-gmt3 clone #9 Allele 2”, “Mutant GDP-Fucose Transporter Sequence—CHO-gmt9 (Clone GalF7) Allele 1”, “Mutant GDP-Fucose Transporter Sequence—CHO-gmt9 (Clone GalF7) Allele 2”, “Mutant GDP-Fucose Transporter Sequence—CHO-gmt9 (Clone GalF102) Allele 1”, “Mutant GDP-Fucose Transporter Sequence—CHO-gmt9 (Clone GalF103) Allele 1”, “Mutant GDP-Fucose Transporter Sequence—CHO-gmt9 (Clone GalF103) Allele 2”, “Mutant GDP-Fucose Transporter Sequence—CHO-gmt9 (Clone GalF109) Allele 1”, “Mutant GDP-Fucose Transporter Sequence—CHO-gmt9 (Clone GalF109) Allele 2” below.

We further provide for cells such as CHO cells comprising such a GDP-fucose transporter (Slc35c1) mutant and their use in expression of proteins such as recombinant polypeptides.

### Mutant UDP-Galactose Transporter and GDP-Fucose Transporter Polypeptides

The CHO cells and cell lines comprise mutant UDP-galactose transporter and/or GDP-fucose transporter polypeptides.

We therefore provide generally for a mutant UDP-galactose transporter and/or GDP-fucose transporter polypeptide, together with fragments, homologues, variants and derivatives thereof. These polypeptide sequences may comprise the polypeptide sequences disclosed here, and particularly in the sequence listings.

The mutant UDP-galactose transporter and/or GDP-fucose transporter polypeptide may comprise one or more changes compared to the wild type UDP-galactose transporter and/or GDP-fucose transporter sequence respectively. Such mutations may result from stop codons being introduced in the encoding nucleic acid sequence and consequent premature termination of translation of the UDP-galactose transporter and/or GDP-fucose transporter mRNA.

The mutant UDP-galactose transporter and/or GDP-fucose transporter polypeptide may be shorter than a respective wild type UDP-galactose transporter and/or GDP-fucose transporter polypeptide. It may be a truncated version of wild type UDP-galactose transporter and/or GDP-fucose transporter polypeptide. The length of the mutant UDP-galactose transporter and/or GDP-fucose transporter polypeptide may be 90% or less, 80% or less, 70% or less, etc than the wild type sequence.

For example, a mutant UDP-galactose transporter and/or GDP-fucose transporter polypeptide may be missing 5, 10, 15, 20, 25, 30, 35, 40, 45, 50, 55, 60, 65, 70, 75, 80, 85, 90, 95, 100 or more C-terminal residues compared to a respective full length or wild type UDP-galactose transporter and/or GDP-fucose transporter polypeptide.

The mutant UDP-galactose transporter and/or GDP-fucose transporter polypeptide may for example comprise a sequence as set out above. The mutant UDP-galactose transporter and/or GDP-fucose transporter polypeptide may comprise a UDP-galactose transporter and/or GDP-fucose transporter sequence comprising a mutation as set out above.

It will be understood that the mutant UDP-galactose transporter and/or GDP-fucose transporter polypeptide sequences disclosed here are not limited to the particular sequences set forth in the sequence listing, or fragments thereof, or sequences obtained from mutant UDP-galactose transporter and/or GDP-fucose transporter protein, but also include homologous sequences obtained from any source, for example related cellular homologues, homologues from other species and variants or derivatives thereof, provided that they have at least one of the biological activities of mutant UDP-galactose transporter and/or GDP-fucose transporter, as the case may be.

This disclosure therefore encompasses variants, homologues or derivatives of the amino acid sequences set forth in the sequence listings, as well as variants, homologues or derivatives of the amino acid sequences encoded by the nucleotide sequences disclosed here. Such a sequences is generally referred to as a “mutant UDP-galactose transporter” or “GDP-fucose transporter sequence”.

The length of the mutant UDP-galactose transporter and/or GDP-fucose transporter polypeptide may be 90% or less, 80% or less, 70% or less, etc than a corresponding wild type sequence.

For example, a mutant UDP-galactose transporter and/or GDP-fucose transporter nucleic acid may encode a mutant UDP-galactose transporter and/or GDP-fucose transporter polypeptide that is missing 5, 10, 15, 20, 25, 30, 35, 40, 45, 50, 55, 60, 65, 70, 75, 80, 85, 90, 95, 100 or more C-terminal residues.

Biological Activities

In some embodiments, the sequences lack at least one biological activity of mutant UDP-galactose transporter and/or GDP-fucose transporter, as the case may be.

The biological activity in relation to UDP-galactose transporter may comprise inability to transport UDP-galactose into the Golgi apparatus compared to wild-type UDP-galactose transporter (Slc35A2). The biological activity in relation to GDP-fucose transporter may comprise inability to transport transport GDP-fucose into the Golgi apparatus compared to wild-type GDP-fucose transporter (Slc35C1).

Identification of UDP-Galactose Transporter Mutants

Mutants carrying dysfunctional UDP-Galactose Transporter may be identified as follow:

CHO cells are mutated by means known in the art. Human erythropoietin (EPO) is transiently expressed in a wild type or a putative mutant CHO cell line. The culture media (supernatant) that contains the secreted EPO is collected. The supernatants are desalted and the EPO molecules in the supernatants are separated by an isoelectric focusing (IEF) gel.

The separated EPO bands on the IEF gel are visualized by Western blot using an anti-EPO antibody. As EPO produced in wild type CHO cells are fully glycosylated with many sialic acid residues, they are highly negatively charged. Therefore, in this IEF-western blot assay, EPO produced by wild type CHO cells are located at the low pH end in a pH3 to pH10 gradient.

However, EPO molecules produced in mutant CHO cells lacking UDP-Galactose Transporter are located to the basic end (high pH end) on the same IEF gel.

A genetic complementation test may be conducted to confirm the result.

If a construct that expresses functional UDP-Galactose Transporter is co-transfected with the EPO construct, the glycosylation of EPO produced becomes completed (i.e., the glycosylation pattern becomes similar to one produced by a wild-type CHO cell).

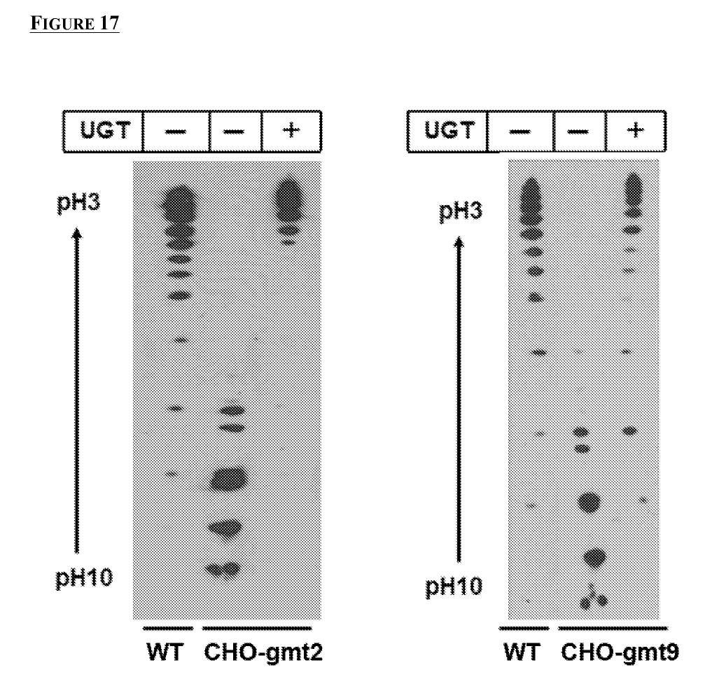

FIG. 17 shows an isoelectric focusing gel of human erythropoietin expressed in wild type and mutant cells. The figure shows CHO-K1 (wild-type) cells (left hand panel, left hand lane), CHO-gmt2 cells lacking functional UDP-Galactose Transporter activity (left hand panel, middle lane marked with “−”), CHO-gmt2 cells complemented with construct that expresses functional UDP-Galactose Transporter (left hand panel, right hand lane marked “+”); CHO-K1 (wild-type) cells (right hand panel, left hand lane), CHO-gmt9 cells lacking functional UDP-Galactose Transporter activity and lacking functional GDP-Fucose Transporter activity (right hand panel, middle lane marked with “−”) and CHO-gmt9 cells complemented with construct that expresses functional UDP-Galactose Transporter (right hand panel, right hand lane marked “+”).

Further reference may be made to Lim et al (2008). The Golgi CMP-sialic acid transporter: A new CHO mutant provides functional insights. Glycobiology 18(11), 851-860.

Identification of GDP-Fucose Transporter Mutants

Mutants carrying dysfunctional GDP-Fucose Transporter may be identified as follow:

CHO cells are mutated by means known in the art. A fucose-specific lectin Aleuria aurantia lectin (AAL) is used to stain putative mutant cells. Wild type CHO cells are stained positively with AAL in a FACS analysis because there are many glycoproteins on the cell surface that contain fucose.

However, in mutant cells lacking GDP-Fucose Transporter there is no fucose on the cell surface. Therefore, AAL stains such cells negatively.

A genetic complementation test may be conducted to confirm the result.

If a construct that expresses functional GDP-Fucose Transporter is transfected into the putative CHO cell lacking GDP-Fucose Transporter identified in the assay above, then the cell stains positive for AAL.

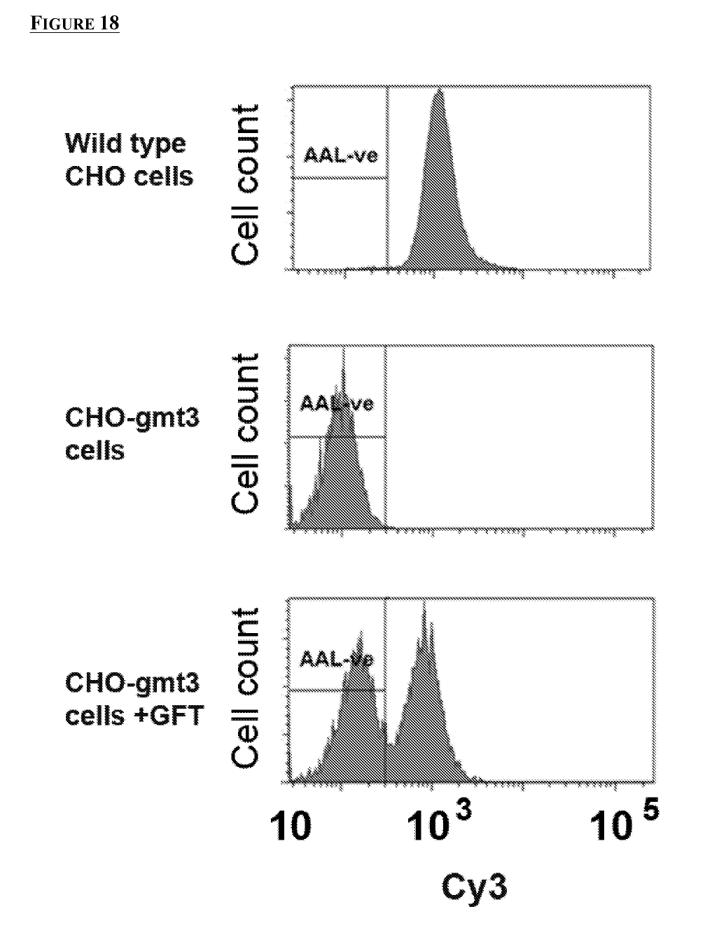

FIG. 18 shows a FACS profile of wild type and mutant cells stained with AAL. The figure shows wild type CHO cells (top panel), CHO-gmt3 cells (middle panel) and CHO-gmt3 cells complemented with a construct expressing functional GDP-fucose transporter (lower panel).

Further reference may be made to Zhang et al (2012). Identification of functional elements of the GDP-fucose transporter SLC35C1 using a novel Chinese hamster ovary mutant. Glycobiology 22(7), 897-911.

Homologues

The polypeptides disclosed include homologous sequences obtained from any source, for example related viral/bacterial proteins, cellular homologues and synthetic peptides, as well as variants or derivatives thereof.

In the context of the present document, a homologous sequence or homologue is taken to include an amino acid sequence which is at least 60, 70, 80 or 90% identical, such as at least 95 or 98% identical at the amino acid level over at least 30, such as 50, 70, 90 or 100 amino acids with UDP-galactose transporter and/or GDP-fucose transporter, as the case may be, for example as shown in the sequence listing herein. In the context of this document, a homologous sequence is taken to include an amino acid sequence which is at least 15, 20, 25, 30, 40, 50, 60, 70, 80 or 90% identical, such as at least 95 or 98% identical at the amino acid level, such as over at least 15, 25, 35, 50 or 100, such as 200, 300, 400 or 500 amino acids with the sequence of UDP-galactose transporter and/or GDP-fucose transporter.

Although homology can also be considered in terms of similarity (i.e. amino acid residues having similar chemical properties/functions), in the context of the present document it is possible to express homology in terms of sequence identity. In some embodiments, the sequence identity is determined relative to the entirety of the length the relevant sequence, i.e., over the entire length or full length sequence of the relevant gene, for example.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, the default values may be used when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). The public default values for the GCG package, or in the case of other software, the default matrix, such as BLOSUM62 may be used.

Once the software has produced an optimal alignment, it is possible to calculate % homology, such as % sequence identity. The software typically does this as part of the sequence comparison and generates a numerical result.

Variants and Derivatives

The terms “variant” or “derivative” in relation to the amino acid sequences as described here includes any substitution of, variation of, modification of, replacement of, deletion of or addition of one (or more) amino acids from or to the sequence. For example, the resultant amino acid sequence retains substantially the same activity as the unmodified sequence, such as having at least the same activity as the mutant UDP-galactose transporter and/or GDP-fucose transporter polypeptide shown in the sequence listings.

Polypeptides having the amino acid sequence shown in the Examples, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Amino acid substitutions may include the use of non-naturally occurring analogues, for example to increase blood plasma half-life of a therapeutically administered polypeptide.

Natural variants of mutant UDP-galactose transporter and/or GDP-fucose transporter are likely to comprise conservative amino acid substitutions. Conservative substitutions may be defined, for example according to the Table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Fragments

Polypeptides disclosed here and useful as markers also include fragments of the above mentioned full length polypeptides and variants thereof, including fragments of the sequences set out in the sequence listings.

Polypeptides also include fragments of the full length sequence of the mutant UDP-galactose transporter and/or GDP-fucose transporter polypeptide. Such fragments may comprise at least one epitope. Methods of identifying epitopes are well known in the art. Fragments will typically comprise at least 6 amino acids, such as at least 10, 20, 30, 50 or 100 amino acids.

Included are fragments comprising, such as consisting of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145 or 150, or more residues from a mutant UDP-galactose transporter and/or GDP-fucose transporter amino acid sequence.

Polypeptide fragments of the mutant UDP-galactose transporter and/or GDP-fucose transporter proteins and allelic and species variants thereof may contain one or more (e.g. 5, 10, 15, or 20) substitutions, deletions or insertions, including conserved substitutions. Where substitutions, deletion and/or insertions occur, for example in different species, such as less than 50%, 40% or 20% of the amino acid residues depicted in the sequence listings are altered.

Mutant UDP-galactose transporter and/or GDP-fucose transporter, and fragments, homologues, variants and derivatives, may be made by recombinant means. However, they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6× His, GAL4 (DNA binding and/or transcriptional activation domains) and β-galactosidase. It may also be convenient to include a proteolytic cleavage site between the fusion protein partner and the protein sequence of interest to allow removal of fusion protein sequences. The fusion protein may be such that it does not hinder the function of the protein of interest sequence. Proteins may also be obtained by purification of cell extracts from animal cells.

The mutant UDP-galactose transporter and/or GDP-fucose transporter polypeptide, variants, homologues, fragments and derivatives disclosed here may be in a substantially isolated form. It will be understood that such polypeptides may be mixed with carriers or diluents which will not interfere with the intended purpose of the protein and still be regarded as substantially isolated. A mutant UDP-galactose transporter and/or GDP-fucose transporter variant, homologue, fragment or derivative may also be in a substantially purified form, in which case it will generally comprise the protein in a preparation in which more than 90%, e.g. 95%, 98% or 99% of the protein in the preparation is a protein.

The mutant UDP-galactose transporter and/or GDP-fucose transporter polypeptides variants, homologues, fragments and derivatives disclosed here may be labelled with a revealing label. The revealing label may be any suitable label which allows the polypeptide, etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols.

Mutant UDP-galactose transporter and/or GDP-fucose transporter polypeptide, variants, homologues, fragments and derivatives disclosed here, optionally labelled, my also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The mutant UDP-galactose transporter and/or GDP-fucose transporter polypeptides variants, homologues, fragments and derivatives disclosed here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which the mutant UDP-galactose transporter and/or GDP-fucose transporter polypeptide, variants, homologues, fragments and derivatives disclosed here are expressed may be used in assay systems to identify candidate substances which interfere with or enhance the functions of the polypeptides in the cell.

Mutant UDP-Galactose Transporter and/or GDP-Fucose Transporter Nucleic Acids

The CHO cells and cell lines comprise mutant UDP-galactose transporter and/or GDP-fucose transporter nucleic acids.

We therefore provide generally for a mutant UDP-galactose transporter and/or GDP-fucose transporter nucleic acid, together with fragments, homologues, variants and derivatives thereof. These nucleic acid sequences may encode the polypeptide sequences disclosed here, and particularly in the sequence listings.

The polynucleotide may comprise a mutant UDP-galactose transporter and/or GDP-fucose transporter nucleic acid. The mutant UDP-galactose transporter and/or GDP-fucose transporter nucleic acid may comprise one or more point mutations in the wild type UDP-galactose transporter and/or GDP-fucose transporter sequence. Such mutations may result in corresponding changes to the amino acid sequence, or introduce stop codons and premature termination of translation of the UDP-galactose transporter and/or GDP-fucose transporter mRNA.

The mutant UDP-galactose transporter and/or GDP-fucose transporter nucleic acid may comprise a mutation resulting in a stop codon, which results in a mutant UDP-galactose transporter and/or GDP-fucose transporter polypeptide being shorter than a wild type UDP-galactose transporter and/or GDP-fucose transporter polypeptide. The length of the mutant UDP-galactose transporter and/or GDP-fucose transporter polypeptide may be 90% or less, 80% or less, 70% or less, etc than the wild type sequence.

For example, a mutant UDP-galactose transporter and/or GDP-fucose transporter nucleic acid may encode a mutant UDP-galactose transporter and/or GDP-fucose transporter polypeptide that is missing 5, 10, 15, 20, 25, 30, 35, 40, 45, 50, 55, 60, 65, 70, 75, 80, 85, 90, 95, 100 or more C-terminal residues.

The mutant UDP-galactose transporter and/or GDP-fucose transporter nucleic acid may for example comprise a sequence set out above. The mutant UDP-galactose transporter and/or GDP-fucose transporter nucleic acid may comprise a UDP-galactose transporter and/or GDP-fucose transporter sequence comprising a mutation set out above.

In particular, we provide for nucleic acids or polynucleotides which encode any of the UDP-galactose transporter and/or GDP-fucose transporter polypeptides disclosed here. Thus, the terms “UDP-galactose transporter” and “GDP-fucose transporter sequence” should be construed accordingly. However, such a nucleic acid or polynucleotide may comprise a sequence set out above and labelled accordingly, or a sequence encoding a of the corresponding polypeptide, and a fragment, homologue, variant or derivative of such a nucleic acid. The above terms therefore may be taken to refer to these sequences.

As used here in this document, the terms “polynucleotide”, “nucleotide”, and nucleic acid are intended to be synonymous with each other. “Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Mutant UDP-Galactose Transporter and/or GDP-Fucose Transporter Variants, Derivatives and Homologues

The mutant UDP-galactose transporter and/or GDP-fucose transporter polynucleotides described here may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present document, it is to be understood that the polynucleotides described herein may be modified by any method available in the art. Such modifications may be carried out in order to enhance the in vivo activity or life span of polynucleotides.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleotides from or to the sequence. The resulting sequence may be capable of encoding a polypeptide which is capable of expressing a biological activity in a CHO cell.

As indicated above, with respect to sequence identity, a “homologue” has for example at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence shown in the sequence listings.

There may be at least 95% identity, such as at least 96% identity, such as at least 97% identity, such as at least 98% identity, such as at least 99% identity. Nucleotide homology comparisons may be conducted as described above. A sequence comparison program that may be used is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

In some embodiments, a mutant UDP-galactose transporter and/or GDP-fucose transporter polynucleotide has at least 90% or more sequence identity to a sequence set out above and labelled accordingly. The mutant UDP-galactose transporter and/or GDP-fucose transporter polynucleotide may have 60% or more, such as 65% or more, 70% or more, 75% or more, 80% or more, 85% or more, 90% or more, 95% or more, 97% or more or 98% or more sequence identity to a sequence set out above and labelled accordingly.

Hybridisation

We further describe mutant UDP-galactose transporter and/or GDP-fucose transporter nucleotide sequences that are capable of hybridising selectively to any of the sequences presented herein, or any variant, fragment or derivative thereof, or to the complement of any of the above. Nucleotide sequences are such as at least 15 nucleotides in length, such as at least 20, 30, 40 or 50 nucleotides in length.

The term “hybridisation” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, will be generally at least 70%, such as at least 80 or 90% or such as at least 95% or 98% homologous to the corresponding nucleotide sequences presented herein over a region of at least 20, such as at least 25 or 30, for instance at least 40, 60 or 100 or more contiguous nucleotides.

The term “selectively hybridisable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screened. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical polynucleotide sequences while an intermediate (or low) stringency hybridisation can be used to identify or detect similar or related polynucleotide sequences.

In a one aspect, we disclose nucleotide sequences that can hybridise to a mutant UDP-galactose transporter and/or GDP-fucose transporter nucleic acid, or a fragment, homologue, variant or derivative thereof, under stringent conditions (e.g. 65° C. and 0.1×SSC {1×SSC=0.15 M NaCl, 0.015 M Na₃ Citrate pH 7.0}).

Where a polynucleotide is double-stranded, both strands of the duplex, either individually or in combination, are encompassed by the present disclosure. Where the polynucleotide is single-stranded, it is to be understood that the complementary sequence of that polynucleotide is also disclosed and encompassed.

Polynucleotides which are not 100% homologous to the sequences disclosed here but fall within the disclosure can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the relevant sequence under conditions of medium to high stringency. Similar considerations apply to obtaining species homologues and allelic variants of mutant UDP-galactose transporter and/or GDP-fucose transporter.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, such as at least 20, for example at least 25, 30 or 40 nucleotides in length, and are also encompassed by the term polynucleotides as used herein. Fragments may be less than 500, 200, 100, 50 or 20 nucleotides in length.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

### Mutant UDP-Galactose Transporter and Mutant GDP-Fucose Transporter Sequences

Mutant UDP-Galactose Transporter—CHO-gmt2

The UDP-Galactose transporter may comprise a T insertion at position 955 of coding sequence, resulting in frameshift and a mutant protein with 430 amino acids.

The mutant UDP-Galactose transporter may comprise a polynucleotide sequence:

ATGGCAGCGGTTGGGGTTGGCGGATCTGCCGCGGCGGCCGGGCCAGGGG
CCGTATCCGCTGGCGCGCTGGAGCCTGGGTCCGCTACAGCGGCTCACCG
GCGCCTCAAGTACATATCCTTAGCTGTGCTGATGGTCCAGAACGCCTCC
CTCATCCTTAGCATCCGATATGCTCGTACACTGCCTGGTGATCGCTTCT
TTGCCACCACCGCTGTGGTCATGGCTGAAGTGCTTAAAGGTGTCACCTG
TCTCCTGCTGCTCTTCGCCCAGAAGAGGGGTAATGTGAAGCACCTGGTT
CTCTTCCTCCACGAGGCTGTCCTGGTGCAATATGTGGACACACTCAAGC
TCGCGGTGCCCTCTCTCATCTATACTTTGCAGAATAACCTCCAGTATGT
TGCCATCTCCAACCTGCCAGCTGCCACTTTCCAGGTGACATATCAGCTC
AAGATCCTGACTACAGCACTGTTCTCCGTGCTCATGTTGAACCGCAGCC
TCTCACGCCTGCAGTGGGCCTCCCTGCTGCTGCTCTTCACTGGTGTGGC
GATTGTCCAGGCACAGCAAGCTGGTGGGGGTGGCCCACGGCCACTAGAT
CAGAATCCCGGGGCCGGACTAGCAGCTGTGGTGGCCTCCTGTCTCTCCT
CAGGCTTTGCAGGGGTATACTTTGAGAAGATCCTCAAAGGCAGCTCAGG
TTCTGTGTGGCTGCGTAACCTCCAACTAGGCCTCTTTGGCACAGCACTG
GGCCTGGTGGGGCTCTGGTGGGCTGAAGGCACTGCTGTGGCCCGTCGAG
GCTTCTTCTTTGGATACACGCCTGCTGTCTGGGGGGTGGTACTAAACCA
AGCCTTTGGTGGGCTACTGGTGGCTGTTGTAGTCAAGTACGCTGACAAC
ATCCTCAAGGGCTTTGCCACCTCCCTGTCTATTGTGCTGTCCACTGTTG
CCTCCATTCGCCTCTTTGGCTTTTCACCTGGACCCATTATTTGCCCTGG
GCGCTGGGCTCGTCATTGGTGCCGTCTACCTCTACAGCCTTCCCCGAAG
TGCAGTCAAAGCCATAACTTCTGCCTCTGCCTCTGCCTCTGCCTCTGGG
CCCTGCATTCACCAGCAGCCTCCTGGGCAGCCACCACCACCACCGCAGC
TGTCTTCTCGAGGAGACCTCATCACGGAGCCCTTTCTGCCAAAGTTGCT
CACCAAGGTGAAGGGTTCGTAGCTGCTGGAATTGAAGACACTGGCCTGC
CTTCGTTCTCCCTCTCTTGCCCTGGCCCAGCTGGGACTAAACTCTTATC
AGTATTAGGGGTAGGGTGA

The mutant UDP-Galactose transporter may comprise a polypeptide sequence:

MAAVGVGGSAAAAGPGAVSAGALEPGSATAAHRRLKYISLAVLMVQNASL
ILSIRYARTLPGDRFFATTAVVMAEVLKGVTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGGGPRPLDQNPG
AGLAAVVASCLSSGFAGVYFEKILKGSSGSVWLRNLQLGLFGTALGLVGL
WWAEGTAVARRGFFFGYTPAVWGVVLNQAFGGLLVAVVVKYADNILKGFA
TSLSIVLSTVASIRLFGFSPGPIICPGRWARHWCRLPLQPSPKCSQSHNF
CLCLCLCLWALHSPAASWAATTTTAAVFSRRPHHGALSAKVAHQGEGFVA
AGIEDTGLPSFSLSCPGPAGTKLLSVLGVG

Mutant GDP-Fucose Transporter Sequence—CHO-gmt3 Clone #2 Allele 1

The GDP-fucose transporter mutation may comprise a deletion of 2 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGCGTAGGGGTGGCCTTCTACAACGTGGGGCGCTCGCTCAC
CACCGTGTTCAATGTGCTTCTGTCCTACCTGCTGCTCAAACAGACCACTT
CCTTCTATGCCCTGCTCACATGTGGCATCATCATTGGTGGTTTCTGGCTG
GGTATAGACCAAGAGGGAGCTGAGGGCACCCTGTCCCTCATAGGCACCAT
CTTCGGGGTGCTGGCCAGCCTCTGCGTCTCCCTCAATGCCATCTATACCA
AGAAGGTGCTCCCAGCAGTGGACAACAGCATCTGGCGCCTAACCTTCTAT
AACAATGTCAATGCCTGTGTGCTCTTCTTGCCCCTGATGGTTCTGCTGGG
TGAGCTCCGTGCCCTCCTTGACTTTGCTCATCTGTACAGTGCCCACTTCT
GGCTCATGATGACGCTGGGTGGCCTCTTCGGCTTTGCCATTGGCTATGTG
ACAGGACTGCAGATCAAATTCACCAGTCCCCTGACCCACAATGTATCAGG
CACAGCCAAGGCCTGTGCGCAGACAGTGCTGGCCGTGCTCTACTATGAAG
AGACTAAGAGCTTCCTGTGGTGGACAAGCAACCTGATGGTGCTGGGTGGC
TCCTCAGCCTATACCTGGGTCAGGGGCTGGGAGATGCAGAAGACCCAAGA
GGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKRRGGLLQRGALAH
HRVQCASVLPAAQTDHFLLCPAHMWHHHWWFLAGYRPRGS

Mutant GDP-Fucose Transporter Sequence—CHO-gmt3 Clone #2 Allele 2

The GDP-fucose transporter mutation may comprise a deletion of 35 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TTGGGGCGCTCGCTCACCACCGTGTTCAATGTGCTTCTGTCCTACCTGCT
GCTCAAACAGACCACTTCCTTCTATGCCCTGCTCACATGTGGCATCATCA
TTGGTGGTTTCTGGCTGGGTATAGACCAAGAGGGAGCTGAGGGCACCCTG
TCCCTCATAGGCACCATCTTCGGGGTGCTGGCCAGCCTCTGCGTCTCCCT
CAATGCCATCTATACCAAGAAGGTGCTCCCAGCAGTGGACAACAGCATCT
GGCGCCTAACCTTCTATAACAATGTCAATGCCTGTGTGCTCTTCTTGCCC
CTGATGGTTCTGCTGGGTGAGCTCCGTGCCCTCCTTGACTTTGCTCATCT
GTACAGTGCCCACTTCTGGCTCATGATGACGCTGGGTGGCCTCTTCGGCT
TTGCCATTGGCTATGTGACAGGACTGCAGATCAAATTCACCAGTCCCCTG
ACCCACAATGTATCAGGCACAGCCAAGGCCTGTGCGCAGACAGTGCTGGC
CGTGCTCTACTATGAAGAGACTAAGAGCTTCCTGTGGTGGACAAGCAACC
TGATGGTGCTGGGTGGCTCCTCAGCCTATACCTGGGTCAGGGGCTGGGAG
ATGCAGAAGACCCAAGAGGACCCCAGCTCCAAAGAGGGTGAGAAGAGTGC
TATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLGALAHHRVQCASVLPA
AQTDHFLLCPAHMWHHHWWFLAGYRPRGS

Mutant GDP-Fucose Transporter Sequence—CHO-gmt3 Clone #3 Allele 1

The GDP-fucose transporter mutation may comprise a deletion of 2 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTATAGGGGTGGCCTTCTACAACGTGGGGCGCTCGCTCAC
CACCGTGTTCAATGTGCTTCTGTCCTACCTGCTGCTCAAACAGACCACTT
CCTTCTATGCCCTGCTCACATGTGGCATCATCATTGGTGGTTTCTGGCTG
GGTATAGACCAAGAGGGAGCTGAGGGCACCCTGTCCCTCATAGGCACCAT
CTTCGGGGTGCTGGCCAGCCTCTGCGTCTCCCTCAATGCCATCTATACCA
AGAAGGTGCTCCCAGCAGTGGACAACAGCATCTGGCGCCTAACCTTCTAT
AACAATGTCAATGCCTGTGTGCTCTTCTTGCCCCTGATGGTTCTGCTGGG
TGAGCTCCGTGCCCTCCTTGACTTTGCTCATCTGTACAGTGCCCACTTCT
GGCTCATGATGACGCTGGGTGGCCTCTTCGGCTTTGCCATTGGCTATGTG
ACAGGACTGCAGATCAAATTCACCAGTCCCCTGACCCACAATGTATCAGG
CACAGCCAAGGCCTGTGCGCAGACAGTGCTGGCCGTGCTCTACTATGAAG
AGACTAAGAGCTTCCTGTGGTGGACAAGCAACCTGATGGTGCTGGGTGGC
TCCTCAGCCTATACCTGGGTCAGGGGCTGGGAGATGCAGAAGACCCAAGA
GGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKYRGGLLQRGALAH
HRVQCASVLPAAQTDHFLLCPAHMWHHHWWFLAGYRPRGS

Mutant GDP-Fucose Transporter Sequence—CHO-gmt3 Clone #3 Allele 2

The GDP-fucose transporter mutation may comprise an insertion of 3 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTAGTACGTAGGGGTGGCCTTCTACAACGTGGGGCGCTCG
CTCACCACCGTGTTCAATGTGCTTCTGTCCTACCTGCTGCTCAAACAGAC
CACTTCCTTCTATGCCCTGCTCACATGTGGCATCATCATTGGTGGTTTCT
GGCTGGGTATAGACCAAGAGGGAGCTGAGGGCACCCTGTCCCTCATAGGC
ACCATCTTCGGGGTGCTGGCCAGCCTCTGCGTCTCCCTCAATGCCATCTA
TACCAAGAAGGTGCTCCCAGCAGTGGACAACAGCATCTGGCGCCTAACCT
TCTATAACAATGTCAATGCCTGTGTGCTCTTCTTGCCCCTGATGGTTCTG
CTGGGTGAGCTCCGTGCCCTCCTTGACTTTGCTCATCTGTACAGTGCCCA
CTTCTGGCTCATGATGACGCTGGGTGGCCTCTTCGGCTTTGCCATTGGCT
ATGTGACAGGACTGCAGATCAAATTCACCAGTCCCCTGACCCACAATGTA
TCAGGCACAGCCAAGGCCTGTGCGCAGACAGTGCTGGCCGTGCTCTACTA
TGAAGAGACTAAGAGCTTCCTGTGGTGGACAAGCAACCTGATGGTGCTGG
GTGGCTCCTCAGCCTATACCTGGGTCAGGGGCTGGGAGATGCAGAAGACC
CAAGAGGACCCCAGCTCCAAAGAGGGTGAGAAGAGTGCTATCAGGGTGTG
A

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLK

Mutant GDP-Fucose Transporter Sequence—CHO-gmt3 Clone #4 Allele 1

The GDP-fucose transporter mutation may comprise a deletion of 2 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGCGTAGGGGTGGCCTTCTACAACGTGGGGCGCTCGCTCAC
CACCGTGTTCAATGTGCTTCTGTCCTACCTGCTGCTCAAACAGACCACTT
CCTTCTATGCCCTGCTCACATGTGGCATCATCATTGGTGGTTTCTGGCTG
GGTATAGACCAAGAGGGAGCTGAGGGCACCCTGTCCCTCATAGGCACCAT
CTTCGGGGTGCTGGCCAGCCTCTGCGTCTCCCTCAATGCCATCTATACCA
AGAAGGTGCTCCCAGCAGTGGACAACAGCATCTGGCGCCTAACCTTCTAT
AACAATGTCAATGCCTGTGTGCTCTTCTTGCCCCTGATGGTTCTGCTGGG
TGAGCTCCGTGCCCTCCTTGACTTTGCTCATCTGTACAGTGCCCACTTCT
GGCTCATGATGACGCTGGGTGGCCTCTTCGGCTTTGCCATTGGCTATGTG
ACAGGACTGCAGATCAAATTCACCAGTCCCCTGACCCACAATGTATCAGG
CACAGCCAAGGCCTGTGCGCAGACAGTGCTGGCCGTGCTCTACTATGAAG
AGACTAAGAGCTTCCTGTGGTGGACAAGCAACCTGATGGTGCTGGGTGGC
TCCTCAGCCTATACCTGGGTCAGGGGCTGGGAGATGCAGAAGACCCAAGA
GGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKRRGGLLQRGALAH
HRVQCASVLPAAQTDHFLLCPAHMWHHHWWFLAGYRPRGS

Mutant GDP-Fucose Transporter Sequence—CHO-gmt3 Clone #4 Allele 2

The GDP-fucose transporter mutation may comprise an insertion of 1 bp resulting in frameshift and premature stop.codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAAGTACGTAGGGGTGGCCTTCTACAACGTGGGGCGCTCGCT
CACCACCGTGTTCAATGTGCTTCTGTCCTACCTGCTGCTCAAACAGACCA
CTTCCTTCTATGCCCTGCTCACATGTGGCATCATCATTGGTGGTTTCTGG
CTGGGTATAGACCAAGAGGGAGCTGAGGGCACCCTGTCCCTCATAGGCAC
CATCTTCGGGGTGCTGGCCAGCCTCTGCGTCTCCCTCAATGCCATCTATA
CCAAGAAGGTGCTCCCAGCAGTGGACAACAGCATCTGGCGCCTAACCTTC
TATAACAATGTCAATGCCTGTGTGCTCTTCTTGCCCCTGATGGTTCTGCT
GGGTGAGCTCCGTGCCCTCCTTGACTTTGCTCATCTGTACAGTGCCCACT
TCTGGCTCATGATGACGCTGGGTGGCCTCTTCGGCTTTGCCATTGGCTAT
GTGACAGGACTGCAGATCAAATTCACCAGTCCCCTGACCCACAATGTATC
AGGCACAGCCAAGGCCTGTGCGCAGACAGTGCTGGCCGTGCTCTACTATG
AAGAGACTAAGAGCTTCCTGTGGTGGACAAGCAACCTGATGGTGCTGGGT
GGCTCCTCAGCCTATACCTGGGTCAGGGGCTGGGAGATGCAGAAGACCCA
AGAGGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKVRRGGLLQRGALA
HHRVQCASVLPAAQTDHFLLCPAHMWHHHWWFLAGYRPRGS

Mutant GDP-Fucose Transporter Sequence—CHO-gmt3 Clone #6 Allele 1

The GDP-fucose transporter mutation may comprise a deletion of 19 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGGCCTTCTACAACGTGGGGCGCTCGCTCACCACCGTGTTCAATGTGC
TTCTGTCCTACCTGCTGCTCAAACAGACCACTTCCTTCTATGCCCTGCTC
ACATGTGGCATCATCATTGGTGGTTTCTGGCTGGGTATAGACCAAGAGGG
AGCTGAGGGCACCCTGTCCCTCATAGGCACCATCTTCGGGGTGCTGGCCA
GCCTCTGCGTCTCCCTCAATGCCATCTATACCAAGAAGGTGCTCCCAGCA
GTGGACAACAGCATCTGGCGCCTAACCTTCTATAACAATGTCAATGCCTG
TGTGCTCTTCTTGCCCCTGATGGTTCTGCTGGGTGAGCTCCGTGCCCTCC
TTGACTTTGCTCATCTGTACAGTGCCCACTTCTGGCTCATGATGACGCTG
GGTGGCCTCTTCGGCTTTGCCATTGGCTATGTGACAGGACTGCAGATCAA
ATTCACCAGTCCCCTGACCCACAATGTATCAGGCACAGCCAAGGCCTGTG
CGCAGACAGTGCTGGCCGTGCTCTACTATGAAGAGACTAAGAGCTTCCTG
TGGTGGACAAGCAACCTGATGGTGCTGGGTGGCTCCTCAGCCTATACCTG
GGTCAGGGGCTGGGAGATGCAGAAGACCCAAGAGGACCCCAGCTCCAAAG
AGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLWPSTTWGARSPPCSMC
FCPTCCSNRPLPSMPCSHVASSLVVSGWV

Mutant GDP-Fucose Transporter Sequence—CHO-gmt3 Clone #8 Allele 1

The GDP-fucose transporter mutation may comprise a deletion of 2 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGCGTAGGGGTGGCCTTCTACAACGTGGGGCGCTCGCTCAC
CACCGTGTTCAATGTGCTTCTGTCCTACCTGCTGCTCAAACAGACCACTT
CCTTCTATGCCCTGCTCACATGTGGCATCATCATTGGTGGTTTCTGGCTG
GGTATAGACCAAGAGGGAGCTGAGGGCACCCTGTCCCTCATAGGCACCAT
CTTCGGGGTGCTGGCCAGCCTCTGCGTCTCCCTCAATGCCATCTATACCA
AGAAGGTGCTCCCAGCAGTGGACAACAGCATCTGGCGCCTAACCTTCTAT
AACAATGTCAATGCCTGTGTGCTCTTCTTGCCCCTGATGGTTCTGCTGGG
TGAGCTCCGTGCCCTCCTTGACTTTGCTCATCTGTACAGTGCCCACTTCT
GGCTCATGATGACGCTGGGTGGCCTCTTCGGCTTTGCCATTGGCTATGTG
ACAGGACTGCAGATCAAATTCACCAGTCCCCTGACCCACAATGTATCAGG
CACAGCCAAGGCCTGTGCGCAGACAGTGCTGGCCGTGCTCTACTATGAAG
AGACTAAGAGCTTCCTGTGGTGGACAAGCAACCTGATGGTGCTGGGTGGC
TCCTCAGCCTATACCTGGGTCAGGGGCTGGGAGATGCAGAAGACCCAAGA
GGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKRRGGLLQRGALAH
HRVQCASVLPAAQTDHFLLCPAHMWHHHWWFLAGYRPRGS

Mutant GDP-Fucose Transporter Sequence—CHO-gmt3 Clone #8 Allele 2

The GDP-fucose transporter mutation may comprise a deletion of 35 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TTGGGGCGCTCGCTCACCACCGTGTTCAATGTGCTTCTGTCCTACCTGCT
GCTCAAACAGACCACTTCCTTCTATGCCCTGCTCACATGTGGCATCATCA
TTGGTGGTTTCTGGCTGGGTATAGACCAAGAGGGAGCTGAGGGCACCCTG
TCCCTCATAGGCACCATCTTCGGGGTGCTGGCCAGCCTCTGCGTCTCCCT
CAATGCCATCTATACCAAGAAGGTGCTCCCAGCAGTGGACAACAGCATCT
GGCGCCTAACCTTCTATAACAATGTCAATGCCTGTGTGCTCTTCTTGCCC
CTGATGGTTCTGCTGGGTGAGCTCCGTGCCCTCCTTGACTTTGCTCATCT
GTACAGTGCCCACTTCTGGCTCATGATGACGCTGGGTGGCCTCTTCGGCT
TTGCCATTGGCTATGTGACAGGACTGCAGATCAAATTCACCAGTCCCCTG
ACCCACAATGTATCAGGCACAGCCAAGGCCTGTGCGCAGACAGTGCTGGC
CGTGCTCTACTATGAAGAGACTAAGAGCTTCCTGTGGTGGACAAGCAACC
TGATGGTGCTGGGTGGCTCCTCAGCCTATACCTGGGTCAGGGGCTGGGAG
ATGCAGAAGACCCAAGAGGACCCCAGCTCCAAAGAGGGTGAGAAGAGTGC
TATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLGALAHHRVQCASVLPA
AQTDHFLLCPAHMWHHHWWFLAGYRPRGS

Mutant GDP-Fucose Transporter Sequence—CHO-gmt3 Clone #9 Allele 1

The GDP-fucose transporter mutation may comprise a deletion of 2 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTATAGGGGTGGCCTTCTACAACGTGGGGCGCTCGCTCAC
CACCGTGTTCAATGTGCTTCTGTCCTACCTGCTGCTCAAACAGACCACTT
CCTTCTATGCCCTGCTCACATGTGGCATCATCATTGGTGGTTTCTGGCTG
GGTATAGACCAAGAGGGAGCTGAGGGCACCCTGTCCCTCATAGGCACCAT
CTTCGGGGTGCTGGCCAGCCTCTGCGTCTCCCTCAATGCCATCTATACCA
AGAAGGTGCTCCCAGCAGTGGACAACAGCATCTGGCGCCTAACCTTCTAT
AACAATGTCAATGCCTGTGTGCTCTTCTTGCCCCTGATGGTTCTGCTGGG
TGAGCTCCGTGCCCTCCTTGACTTTGCTCATCTGTACAGTGCCCACTTCT
GGCTCATGATGACGCTGGGTGGCCTCTTCGGCTTTGCCATTGGCTATGTG
ACAGGACTGCAGATCAAATTCACCAGTCCCCTGACCCACAATGTATCAGG
CACAGCCAAGGCCTGTGCGCAGACAGTGCTGGCCGTGCTCTACTATGAAG
AGACTAAGAGCTTCCTGTGGTGGACAAGCAACCTGATGGTGCTGGGTGGC
TCCTCAGCCTATACCTGGGTCAGGGGCTGGGAGATGCAGAAGACCCAAGA
GGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKYRGGLLQRGALAH
HRVQCASVLPAAQTDHFLLCPAHMWHHHWWFLAGYRPRGS

Mutant GDP-Fucose Transporter Sequence—CHO-gmt3 Clone #9 Allele 2

The GDP-fucose transporter mutation may comprise an insertion of 108 bp resulting in frameshift and premature stop codon.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAGGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTAGGATGTGCTTGGTCCGGGAAGACCTGTCACTGAAGTT
ACGCATGCAGATTCGACACTGGAAGGGCCTCTCAGCTGTCGTGCCAGCTG
CATTAATGAATCGGCCAAGTACGTAGGGGTGGCCTTCTACAACGTGGGGC
GCTCGCTCACCACCGTGTTCAATGTGCTTCTGTCCTACCTGCTGCTCAAA
CAGACCACTTCCTTCTATGCCCTGCTCACATGTGGCATCATCATTGGTGG
TTTCTGGCTGGGTATAGACCAAGAGGGAGCTGAGGGCACCCTGTCCCTCA
TAGGCACCATCTTCGGGGTGCTGGCCAGCCTCTGCGTCTCCCTCAATGCC
ATCTATACCAAGAAGGTGCTCCCAGCAGTGGACAACAGCATCTGGCGCCT
AACCTTCTATAACAATGTCAATGCCTGTGTGCTCTTCTTGCCCCTGATGG
TTCTGCTGGGTGAGCTCCGTGCCCTCCTTGACTTTGCTCATCTGTACAGT
GCCCACTTCTGGCTCATGATGACGCTGGGTGGCCTCTTCGGCTTTGCCAT
TGGCTATGTGACAGGACTGCAGATCAAATTCACCAGTCCCCTGACCCACA
ATGTATCAGGCACAGCCAAGGCCTGTGCGCAGACAGTGCTGGCCGTGCTC
TACTATGAAGAGACTAAGAGCTTCCTGTGGTGGACAAGCAACCTGATGGT
GCTGGGTGGCTCCTCAGCCTATACCTGGGTCAGGGGCTGGGAGATGCAGA
AGACCCAAGAGGACCCCAGCTCCAAAGAGGGTGAGAAGAGTGCTATCAGG
GTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLK

Mutant GDP-Fucose Transporter Sequence—CHO-gmt9 (Clone GalF7) Allele 1

The GDP-fucose transporter mutation may comprise an insertion of 3 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTAGTACGTAGGGGTGGCCTTCTACAACGTGGGGCGCTCG
CTCACCACCGTGTTCAATGTGCTTCTGTCCTACCTGCTGCTCAAACAGAC
CACTTCCTTCTATGCCCTGCTCACATGTGGCATCATCATTGGTGGTTTCT
GGCTGGGTATAGACCAAGAGGGAGCTGAGGGCACCCTGTCCCTCATAGGC
ACCATCTTCGGGGTGCTGGCCAGCCTCTGCGTCTCCCTCAATGCCATCTA
TACCAAGAAGGTGCTCCCAGCAGTGGACAACAGCATCTGGCGCCTAACCT
TCTATAACAATGTCAATGCCTGTGTGCTCTTCTTGCCCCTGATGGTTCTG
CTGGGTGAGCTCCGTGCCCTCCTTGACTTTGCTCATCTGTACAGTGCCCA
CTTCTGGCTCATGATGACGCTGGGTGGCCTCTTCGGCTTTGCCATTGGCT
ATGTGACAGGACTGCAGATCAAATTCACCAGTCCCCTGACCCACAATGTA
TCAGGCACAGCCAAGGCCTGTGCGCAGACAGTGCTGGCCGTGCTCTACTA
TGAAGAGACTAAGAGCTTCCTGTGGTGGACAAGCAACCTGATGGTGCTGG
GTGGCTCCTCAGCCTATACCTGGGTCAGGGGCTGGGAGATGCAGAAGACC
CAAGAGGACCCCAGCTCCAAAGAGGGTGAGAAGAGTGCTATCAGGGTGTG
A

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLK

Mutant GDP-Fucose Transporter Sequence—CHO-gmt9 (Clone GalF7) Allele 2

The GDP-fucose transporter mutation may comprise an insertion of 4 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTAAGTACGTAGGGGTGGCCTTCTACAACGTGGGGCGCTC
GCTCACCACCGTGTTCAATGTGCTTCTGTCCTACCTGCTGCTCAAACAGA
CCACTTCCTTCTATGCCCTGCTCACATGTGGCATCATCATTGGTGGTTTC
TGGCTGGGTATAGACCAAGAGGGAGCTGAGGGCACCCTGTCCCTCATAGG
CACCATCTTCGGGGTGCTGGCCAGCCTCTGCGTCTCCCTCAATGCCATCT
ATACCAAGAAGGTGCTCCCAGCAGTGGACAACAGCATCTGGCGCCTAACC
TTCTATAACAATGTCAATGCCTGTGTGCTCTTCTTGCCCCTGATGGTTCT
GCTGGGTGAGCTCCGTGCCCTCCTTGACTTTGCTCATCTGTACAGTGCCC
ACTTCTGGCTCATGATGACGCTGGGTGGCCTCTTCGGCTTTGCCATTGGC
TATGTGACAGGACTGCAGATCAAATTCACCAGTCCCCTGACCCACAATGT
ATCAGGCACAGCCAAGGCCTGTGCGCAGACAGTGCTGGCCGTGCTCTACT
ATGAAGAGACTAAGAGCTTCCTGTGGTGGACAAGCAACCTGATGGTGCTG
GGTGGCTCCTCAGCCTATACCTGGGTCAGGGGCTGGGAGATGCAGAAGAC
CCAAGAGGACCCCAGCTCCAAAGAGGGTGAGAAGAGTGCTATCAGGGTGT
GA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLK

Mutant UDP-Galactose Transporter Sequence—CHO-gmt9 (Clone GalF7)

The UDP-Galactose transporter may comprise a T insertion at position 955 of coding sequence, resulting in frameshift and a mutant protein with 430 amino acids.

The mutant UDP-Galactose transporter may comprise a polynucleotide sequence:

ATGGCAGCGGTTGGGGTTGGCGGATCTGCCGCGGCGGCCGGGCCAGGGGC
CGTATCCGCTGGCGCGCTGGAGCCTGGGTCCGCTACAGCGGCTCACCGGC
GCCTCAAGTACATATCCTTAGCTGTGCTGATGGTCCAGAACGCCTCCCTC
ATCCTTAGCATCCGATATGCTCGTACACTGCCTGGTGATCGCTTCTTTGC
CACCACCGCTGTGGTCATGGCTGAAGTGCTTAAAGGTGTCACCTGTCTCC
TGCTGCTCTTCGCCCAGAAGAGGGGTAATGTGAAGCACCTGGTTCTCTTC
CTCCACGAGGCTGTCCTGGTGCAATATGTGGACACACTCAAGCTCGCGGT
GCCCTCTCTCATCTATACTTTGCAGAATAACCTCCAGTATGTTGCCATCT
CCAACCTGCCAGCTGCCACTTTCCAGGTGACATATCAGCTCAAGATCCTG
ACTACAGCACTGTTCTCCGTGCTCATGTTGAACCGCAGCCTCTCACGCCT
GCAGTGGGCCTCCCTGCTGCTGCTCTTCACTGGTGTGGCGATTGTCCAGG
CACAGCAAGCTGGTGGGGGTGGCCCACGGCCACTAGATCAGAATCCCGGG
GCCGGACTAGCAGCTGTGGTGGCCTCCTGTCTCTCCTCAGGCTTTGCAGG
GGTATACTTTGAGAAGATCCTCAAAGGCAGCTCAGGTTCTGTGTGGCTGC
GTAACCTCCAACTAGGCCTCTTTGGCACAGCACTGGGCCTGGTGGGGCTC
TGGTGGGCTGAAGGCACTGCTGTGGCCCGTCGAGGCTTCTTCTTTGGATA
CACGCCTGCTGTCTGGGGGGTGGTACTAAACCAAGCCTTTGGTGGGCTAC
TGGTGGCTGTTGTAGTCAAGTACGCTGACAACATCCTCAAGGGCTTTGCC
ACCTCCCTGTCTATTGTGCTGTCCACTGTTGCCTCCATTCGCCTCTTTGG
CTTTTCACCTGGACCCATTATTTGCCCTGGGCGCTGGGCTCGTCATTGGT
GCCGTCTACCTCTACAGCCTTCCCCGAAGTGCAGTCAAAGCCATAACTTC
TGCCTCTGCCTCTGCCTCTGCCTCTGGGCCCTGCATTCACCAGCAGCCTC
CTGGGCAGCCACCACCACCACCGCAGCTGTCTTCTCGAGGAGACCTCATC
ACGGAGCCCTTTCTGCCAAAGTTGCTCACCAAGGTGAAGGGTTCGTAGCT
GCTGGAATTGAAGACACTGGCCTGCCTTCGTTCTCCCTCTCTTGCCCTGG
CCCAGCTGGGACTAAACTCTTATCAGTATTAGGGGTAGGGTGA

The mutant UDP-Galactose transporter may comprise a polypeptide sequence:

MAAVGVGGSAAAAGPGAVSAGALEPGSATAAHRRLKYISLAVLMVQNASL
ILSIRYARTLPGDRFFATTAVVMAEVLKGVTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGGGPRPLDQNPG
AGLAAVVASCLSSGFAGVYFEKILKGSSGSVWLRNLQLGLFGTALGLVGL
WWAEGTAVARRGFFFGYTPAVWGVVLNQAFGGLLVAVVVKYADNILKGFA
TSLSIVLSTVASIRLFGFSPGPIICPGRWARHWCRLPLQPSPKCSQSHNF
CLCLCLCLWALHSPAASWAATTTTAAVFSRRPHHGALSAKVAHQGEGFVA
AGIEDTGLPSFSLSCPGPAGTKLLSVLGVG

Mutant GDP-Fucose Transporter Sequence—CHO-gmt9 (Clone GalF102) Allele 1

The GDP-fucose transporter mutation may comprise a deletion of 22 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCACAACGTGGGGCGCTCGCTCACCACCGTGTTCAATGTGCTTC
TGTCCTACCTGCTGCTCAAACAGACCACTTCCTTCTATGCCCTGCTCACA
TGTGGCATCATCATTGGTGGTTTCTGGCTGGGTATAGACCAAGAGGGAGC
TGAGGGCACCCTGTCCCTCATAGGCACCATCTTCGGGGTGCTGGCCAGCC
TCTGCGTCTCCCTCAATGCCATCTATACCAAGAAGGTGCTCCCAGCAGTG
GACAACAGCATCTGGCGCCTAACCTTCTATAACAATGTCAATGCCTGTGT
GCTCTTCTTGCCCCTGATGGTTCTGCTGGGTGAGCTCCGTGCCCTCCTTG
ACTTTGCTCATCTGTACAGTGCCCACTTCTGGCTCATGATGACGCTGGGT
GGCCTCTTCGGCTTTGCCATTGGCTATGTGACAGGACTGCAGATCAAATT
CACCAGTCCCCTGACCCACAATGTATCAGGCACAGCCAAGGCCTGTGCGC
AGACAGTGCTGGCCGTGCTCTACTATGAAGAGACTAAGAGCTTCCTGTGG
TGGACAAGCAACCTGATGGTGCTGGGTGGCTCCTCAGCCTATACCTGGGT
CAGGGGCTGGGAGATGCAGAAGACCCAAGAGGACCCCAGCTCCAAAGAGG
GTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLTTWGARSPPCSMCF
CPTCCSNRPLPSMPCSHVASSLVVSGWV

Mutant UDP-Galactose Transporter Sequence—CHO-gmt9 (Clone GalF102)

The UDP-Galactose transporter may comprise a T insertion at position 955 of coding sequence, resulting in frameshift and a mutant protein with 430 amino acids.

The mutant UDP-Galactose transporter may comprise a polynucleotide sequence:

ATGGCAGCGGTTGGGGTTGGCGGATCTGCCGCGGCGGCCGGGCCAGGGGC
CGTATCCGCTGGCGCGCTGGAGCCTGGGTCCGCTACAGCGGCTCACCGGC
GCCTCAAGTACATATCCTTAGCTGTGCTGATGGTCCAGAACGCCTCCCTC
ATCCTTAGCATCCGATATGCTCGTACACTGCCTGGTGATCGCTTCTTTGC
CACCACCGCTGTGGTCATGGCTGAAGTGCTTAAAGGTGTCACCTGTCTCC
TGCTGCTCTTCGCCCAGAAGAGGGGTAATGTGAAGCACCTGGTTCTCTTC
CTCCACGAGGCTGTCCTGGTGCAATATGTGGACACACTCAAGCTCGCGGT
GCCCTCTCTCATCTATACTTTGCAGAATAACCTCCAGTATGTTGCCATCT
CCAACCTGCCAGCTGCCACTTTCCAGGTGACATATCAGCTCAAGATCCTG
ACTACAGCACTGTTCTCCGTGCTCATGTTGAACCGCAGCCTCTCACGCCT
GCAGTGGGCCTCCCTGCTGCTGCTCTTCACTGGTGTGGCGATTGTCCAGG
CACAGCAAGCTGGTGGGGGTGGCCCACGGCCACTAGATCAGAATCCCGGG
GCCGGACTAGCAGCTGTGGTGGCCTCCTGTCTCTCCTCAGGCTTTGCAGG
GGTATACTTTGAGAAGATCCTCAAAGGCAGCTCAGGTTCTGTGTGGCTGC
GTAACCTCCAACTAGGCCTCTTTGGCACAGCACTGGGCCTGGTGGGGCTC
TGGTGGGCTGAAGGCACTGCTGTGGCCCGTCGAGGCTTCTTCTTTGGATA
CACGCCTGCTGTCTGGGGGGTGGTACTAAACCAAGCCTTTGGTGGGCTAC
TGGTGGCTGTTGTAGTCAAGTACGCTGACAACATCCTCAAGGGCTTTGCC
ACCTCCCTGTCTATTGTGCTGTCCACTGTTGCCTCCATTCGCCTCTTTGG
CTTTTCACCTGGACCCATTATTTGCCCTGGGCGCTGGGCTCGTCATTGGT
GCCGTCTACCTCTACAGCCTTCCCCGAAGTGCAGTCAAAGCCATAACTTC
TGCCTCTGCCTCTGCCTCTGCCTCTGGGCCCTGCATTCACCAGCAGCCTC
CTGGGCAGCCACCACCACCACCGCAGCTGTCTTCTCGAGGAGACCTCATC
ACGGAGCCCTTTCTGCCAAAGTTGCTCACCAAGGTGAAGGGTTCGTAGCT
GCTGGAATTGAAGACACTGGCCTGCCTTCGTTCTCCCTCTCTTGCCCTGG
CCCAGCTGGGACTAAACTCTTATCAGTATTAGGGGTAGGGTGA

The mutant UDP-Galactose transporter may comprise a polypeptide sequence:

MAAVGVGGSAAAAGPGAVSAGALEPGSATAAHRRLKYISLAVLMVQNASL
ILSIRYARTLPGDRFFATTAVVMAEVLKGVTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGGGPRPLDQNPG
AGLAAVVASCLSSGFAGVYFEKILKGSSGSVWLRNLQLGLFGTALGLVGL
WWAEGTAVARRGFFFGYTPAVWGVVLNQAFGGLLVAVVVKYADNILKGFA
TSLSIVLSTVASIRLFGFSPGPIICPGRWARHWCRLPLQPSPKCSQSHNF
CLCLCLCLWALHSPAASWAATTTTAAVFSRRPHHGALSAKVAHQGEGFVA
AGIEDTGLPSFSLSCPGPAGTKLLSVLGVG

Mutant GDP-Fucose Transporter Sequence—CHO-gmt9 (Clone GalF103) Allele 1

The GDP-fucose transporter mutation may comprise a deletion of 5 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTACGTAGGGGTGGCCTTCTACAACGTGGGGCGCTCGCTCACCAC
CGTGTTCAATGTGCTTCTGTCCTACCTGCTGCTCAAACAGACCACTTCCT
TCTATGCCCTGCTCACATGTGGCATCATCATTGGTGGTTTCTGGCTGGGT
ATAGACCAAGAGGGAGCTGAGGGCACCCTGTCCCTCATAGGCACCATCTT
CGGGGTGCTGGCCAGCCTCTGCGTCTCCCTCAATGCCATCTATACCAAGA
AGGTGCTCCCAGCAGTGGACAACAGCATCTGGCGCCTAACCTTCTATAAC
AATGTCAATGCCTGTGTGCTCTTCTTGCCCCTGATGGTTCTGCTGGGTGA
GCTCCGTGCCCTCCTTGACTTTGCTCATCTGTACAGTGCCCACTTCTGGC
TCATGATGACGCTGGGTGGCCTCTTCGGCTTTGCCATTGGCTATGTGACA
GGACTGCAGATCAAATTCACCAGTCCCCTGACCCACAATGTATCAGGCAC
AGCCAAGGCCTGTGCGCAGACAGTGCTGGCCGTGCTCTACTATGAAGAGA
CTAAGAGCTTCCTGTGGTGGACAAGCAACCTGATGGTGCTGGGTGGCTCC
TCAGCCTATACCTGGGTCAGGGGCTGGGAGATGCAGAAGACCCAAGAGGA
CCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLRRGGLLQRGALAHH
RVQCASVLPAAQTDHFLLCPAHMWHHHWWFLAGYRPRGS

Mutant GDP-Fucose Transporter Sequence—CHO-gmt9 (Clone GalF103) Allele 2

The GDP-fucose transporter mutation may comprise an insertion of 4 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAGTAAGTACGTAGGGGTGGCCTTCTACAACGTGGGGCGCTC
GCTCACCACCGTGTTCAATGTGCTTCTGTCCTACCTGCTGCTCAAACAGA
CCACTTCCTTCTATGCCCTGCTCACATGTGGCATCATCATTGGTGGTTTC
TGGCTGGGTATAGACCAAGAGGGAGCTGAGGGCACCCTGTCCCTCATAGG
CACCATCTTCGGGGTGCTGGCCAGCCTCTGCGTCTCCCTCAATGCCATCT
ATACCAAGAAGGTGCTCCCAGCAGTGGACAACAGCATCTGGCGCCTAACC
TTCTATAACAATGTCAATGCCTGTGTGCTCTTCTTGCCCCTGATGGTTCT
GCTGGGTGAGCTCCGTGCCCTCCTTGACTTTGCTCATCTGTACAGTGCCC
ACTTCTGGCTCATGATGACGCTGGGTGGCCTCTTCGGCTTTGCCATTGGC
TATGTGACAGGACTGCAGATCAAATTCACCAGTCCCCTGACCCACAATGT
ATCAGGCACAGCCAAGGCCTGTGCGCAGACAGTGCTGGCCGTGCTCTACT
ATGAAGAGACTAAGAGCTTCCTGTGGTGGACAAGCAACCTGATGGTGCTG
GGTGGCTCCTCAGCCTATACCTGGGTCAGGGGCTGGGAGATGCAGAAGAC
CCAAGAGGACCCCAGCTCCAAAGAGGGTGAGAAGAGTGCTATCAGGGTGT
GA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLK

Mutant UDP-Galactose Transporter Sequence—CHO-gmt9 (Clone GalF103)

The UDP-Galactose transporter may comprise a T insertion at position 955 of coding sequence, resulting in frameshift and a mutant protein with 430 amino acids.

The mutant UDP-Galactose transporter may comprise a polynucleotide sequence:

ATGGCAGCGGTTGGGGTTGGCGGATCTGCCGCGGCGGCCGGGCCAGGGGC
CGTATCCGCTGGCGCGCTGGAGCCTGGGTCCGCTACAGCGGCTCACCGGC
GCCTCAAGTACATATCCTTAGCTGTGCTGATGGTCCAGAACGCCTCCCTC
ATCCTTAGCATCCGATATGCTCGTACACTGCCTGGTGATCGCTTCTTTGC
CACCACCGCTGTGGTCATGGCTGAAGTGCTTAAAGGTGTCACCTGTCTCC
TGCTGCTCTTCGCCCAGAAGAGGGGTAATGTGAAGCACCTGGTTCTCTTC
CTCCACGAGGCTGTCCTGGTGCAATATGTGGACACACTCAAGCTCGCGGT
GCCCTCTCTCATCTATACTTTGCAGAATAACCTCCAGTATGTTGCCATCT
CCAACCTGCCAGCTGCCACTTTCCAGGTGACATATCAGCTCAAGATCCTG
ACTACAGCACTGTTCTCCGTGCTCATGTTGAACCGCAGCCTCTCACGCCT
GCAGTGGGCCTCCCTGCTGCTGCTCTTCACTGGTGTGGCGATTGTCCAGG
CACAGCAAGCTGGTGGGGGTGGCCCACGGCCACTAGATCAGAATCCCGGG
GCCGGACTAGCAGCTGTGGTGGCCTCCTGTCTCTCCTCAGGCTTTGCAGG
GGTATACTTTGAGAAGATCCTCAAAGGCAGCTCAGGTTCTGTGTGGCTGC
GTAACCTCCAACTAGGCCTCTTTGGCACAGCACTGGGCCTGGTGGGGCTC
TGGTGGGCTGAAGGCACTGCTGTGGCCCGTCGAGGCTTCTTCTTTGGATA
CACGCCTGCTGTCTGGGGGGTGGTACTAAACCAAGCCTTTGGTGGGCTAC
TGGTGGCTGTTGTAGTCAAGTACGCTGACAACATCCTCAAGGGCTTTGCC
ACCTCCCTGTCTATTGTGCTGTCCACTGTTGCCTCCATTCGCCTCTTTGG
CTTTTCACCTGGACCCATTATTTGCCCTGGGCGCTGGGCTCGTCATTGGT
GCCGTCTACCTCTACAGCCTTCCCCGAAGTGCAGTCAAAGCCATAACTTC
TGCCTCTGCCTCTGCCTCTGCCTCTGGGCCCTGCATTCACCAGCAGCCTC
CTGGGCAGCCACCACCACCACCGCAGCTGTCTTCTCGAGGAGACCTCATC
ACGGAGCCCTTTCTGCCAAAGTTGCTCACCAAGGTGAAGGGTTCGTAGCT
GCTGGAATTGAAGACACTGGCCTGCCTTCGTTCTCCCTCTCTTGCCCTGG
CCCAGCTGGGACTAAACTCTTATCAGTATTAGGGGTAGGGTGA

The mutant UDP-Galactose transporter may comprise a polypeptide sequence:

MAAVGVGGSAAAAGPGAVSAGALEPGSATAAHRRLKYISLAVLMVQNASL
ILSIRYARTLPGDRFFATTAVVMAEVLKGVTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGGGPRPLDQNPG
AGLAAVVASCLSSGFAGVYFEKILKGSSGSVWLRNLQLGLFGTALGLVGL
WWAEGTAVARRGFFFGYTPAVWGVVLNQAFGGLLVAVVVKYADNILKGFA
TSLSIVLSTVASIRLFGFSPGPIICPGRWARHWCRLPLQPSPKCSQSHNF
CLCLCLCLWALHSPAASWAATTTTAAVFSRRPHHGALSAKVAHQGEGFVA
AGIEDTGLPSFSLSCPGPAGTKLLSVLGVG

Mutant GDP-Fucose Transporter Sequence—CHO-gmt9 (Clone GalF109) Allele 1

The GDP-fucose transporter mutation may comprise an insertion of 1 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGCCTCAAAGTACGTAGGGGTGGCCTTCTACAACGTGGGGCGCTCGCT
CACCACCGTGTTCAATGTGCTTCTGTCCTACCTGCTGCTCAAACAGACCA
CTTCCTTCTATGCCCTGCTCACATGTGGCATCATCATTGGTGGTTTCTGG
CTGGGTATAGACCAAGAGGGAGCTGAGGGCACCCTGTCCCTCATAGGCAC
CATCTTCGGGGTGCTGGCCAGCCTCTGCGTCTCCCTCAATGCCATCTATA
CCAAGAAGGTGCTCCCAGCAGTGGACAACAGCATCTGGCGCCTAACCTTC
TATAACAATGTCAATGCCTGTGTGCTCTTCTTGCCCCTGATGGTTCTGCT
GGGTGAGCTCCGTGCCCTCCTTGACTTTGCTCATCTGTACAGTGCCCACT
TCTGGCTCATGATGACGCTGGGTGGCCTCTTCGGCTTTGCCATTGGCTAT
GTGACAGGACTGCAGATCAAATTCACCAGTCCCCTGACCCACAATGTATC
AGGCACAGCCAAGGCCTGTGCGCAGACAGTGCTGGCCGTGCTCTACTATG
AAGAGACTAAGAGCTTCCTGTGGTGGACAAGCAACCTGATGGTGCTGGGT
GGCTCCTCAGCCTATACCTGGGTCAGGGGCTGGGAGATGCAGAAGACCCA
AGAGGACCCCAGCTCCAAAGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCLKVRRGGLLQRGALA
HHRVQCASVLPAAQTDHFLLCPAHMWHHHWWFLAGYRPRGS

Mutant GDP-Fucose Transporter Sequence—CHO-gmt9 (Clone GalF109) Allele 2

The GDP-fucose transporter mutation may comprise a deletion of 17 bp.

The mutant GDP-fucose transporter may comprise a polynucleotide sequence:

ATGAACAGGGCGCCTCTGAAGCGGTCCAGGATCCTGCGCATGGCGCTGAC
TGGAGGCTCCACTGCCTCTGAGGAGGCAGATGAAGACAGCAGGAACAAGC
CGTTTCTGCTGCGGGCGCTGCAGATCGCGCTGGTCGTCTCTCTCTACTGG
GTCACCTCCATCTCCATGGTATTCCTCAACAAGTACCTGCTGGACAGCCC
CTCCCTGCAGCTGGATACCCCTATCTTCGTCACTTTCTACCAATGCCTGG
TGACCTCTCTGCTGTGCAAGGGCCTCAGCACTCTGGCCACCTGCTGCCCT
GGCACCGTTGACTTCCCCACCCTGAACCTGGACCTTAAGGTGGCCCGCAG
CGTGCTGCCACTGTCGGTAGTCTTCATTGGCATGATAAGTTTCAATAACC
TCTGTGGCCTTCTACAACGTGGGGCGCTCGCTCACCACCGTGTTCAATGT
GCTTCTGTCCTACCTGCTGCTCAAACAGACCACTTCCTTCTATGCCCTGC
TCACATGTGGCATCATCATTGGTGGTTTCTGGCTGGGTATAGACCAAGAG
GGAGCTGAGGGCACCCTGTCCCTCATAGGCACCATCTTCGGGGTGCTGGC
CAGCCTCTGCGTCTCCCTCAATGCCATCTATACCAAGAAGGTGCTCCCAG
CAGTGGACAACAGCATCTGGCGCCTAACCTTCTATAACAATGTCAATGCC
TGTGTGCTCTTCTTGCCCCTGATGGTTCTGCTGGGTGAGCTCCGTGCCCT
CCTTGACTTTGCTCATCTGTACAGTGCCCACTTCTGGCTCATGATGACGC
TGGGTGGCCTCTTCGGCTTTGCCATTGGCTATGTGACAGGACTGCAGATC
AAATTCACCAGTCCCCTGACCCACAATGTATCAGGCACAGCCAAGGCCTG
TGCGCAGACAGTGCTGGCCGTGCTCTACTATGAAGAGACTAAGAGCTTCC
TGTGGTGGACAAGCAACCTGATGGTGCTGGGTGGCTCCTCAGCCTATACC
TGGGTCAGGGGCTGGGAGATGCAGAAGACCCAAGAGGACCCCAGCTCCAA
AGAGGGTGAGAAGAGTGCTATCAGGGTGTGA

The mutant GDP-fucose transporter may comprise a polypeptide sequence:

MNRAPLKRSRILRMALTGGSTASEEADEDSRNKPFLLRALQIALVVSLYW
VTSISMVFLNKYLLDSPSLQLDTPIFVTFYQCLVTSLLCKGLSTLATCCP
GTVDFPTLNLDLKVARSVLPLSVVFIGMISFNNLCGLLQRGALAHHRVQC
ASVLPAAQTDHFLLCPAHMWHHHWWFLAGYRPRGS

Mutant UDP-Galactose Transporter Sequence—CHO-gmt9 (Clone GalF109)

The UDP-Galactose transporter may comprise a T insertion at position 955 of coding sequence, resulting in frameshift and a mutant protein with 430 amino acids.

The mutant UDP-Galactose transporter may comprise a polynucleotide sequence:

ATGGCAGCGGTTGGGGTTGGCGGATCTGCCGCGGCGGCCGGGCCAGGGGC
CGTATCCGCTGGCGCGCTGGAGCCTGGGTCCGCTACAGCGGCTCACCGGC
GCCTCAAGTACATATCCTTAGCTGTGCTGATGGTCCAGAACGCCTCCCTC
ATCCTTAGCATCCGATATGCTCGTACACTGCCTGGTGATCGCTTCTTTGC
CACCACCGCTGTGGTCATGGCTGAAGTGCTTAAAGGTGTCACCTGTCTCC
TGCTGCTCTTCGCCCAGAAGAGGGGTAATGTGAAGCACCTGGTTCTCTTC
CTCCACGAGGCTGTCCTGGTGCAATATGTGGACACACTCAAGCTCGCGGT
GCCCTCTCTCATCTATACTTTGCAGAATAACCTCCAGTATGTTGCCATCT
CCAACCTGCCAGCTGCCACTTTCCAGGTGACATATCAGCTCAAGATCCTG
ACTACAGCACTGTTCTCCGTGCTCATGTTGAACCGCAGCCTCTCACGCCT
GCAGTGGGCCTCCCTGCTGCTGCTCTTCACTGGTGTGGCGATTGTCCAGG
CACAGCAAGCTGGTGGGGGTGGCCCACGGCCACTAGATCAGAATCCCGGG
GCCGGACTAGCAGCTGTGGTGGCCTCCTGTCTCTCCTCAGGCTTTGCAGG
GGTATACTTTGAGAAGATCCTCAAAGGCAGCTCAGGTTCTGTGTGGCTGC
GTAACCTCCAACTAGGCCTCTTTGGCACAGCACTGGGCCTGGTGGGGCTC
TGGTGGGCTGAAGGCACTGCTGTGGCCCGTCGAGGCTTCTTCTTTGGATA
CACGCCTGCTGTCTGGGGGGTGGTACTAAACCAAGCCTTTGGTGGGCTAC
TGGTGGCTGTTGTAGTCAAGTACGCTGACAACATCCTCAAGGGCTTTGCC
ACCTCCCTGTCTATTGTGCTGTCCACTGTTGCCTCCATTCGCCTCTTTGG
CTTTTCACCTGGACCCATTATTTGCCCTGGGCGCTGGGCTCGTCATTGGT
GCCGTCTACCTCTACAGCCTTCCCCGAAGTGCAGTCAAAGCCATAACTTC
TGCCTCTGCCTCTGCCTCTGCCTCTGGGCCCTGCATTCACCAGCAGCCTC
CTGGGCAGCCACCACCACCACCGCAGCTGTCTTCTCGAGGAGACCTCATC
ACGGAGCCCTTTCTGCCAAAGTTGCTCACCAAGGTGAAGGGTTCGTAGCT
GCTGGAATTGAAGACACTGGCCTGCCTTCGTTCTCCCTCTCTTGCCCTGG
CCCAGCTGGGACTAAACTCTTATCAGTATTAGGGGTAGGGTGA

The mutant UDP-Galactose transporter may comprise a polypeptide sequence:

MAAVGVGGSAAAAGPGAVSAGALEPGSATAAHRRLKYISLAVLMVQNASL
ILSIRYARTLPGDRFFATTAVVMAEVLKGVTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGGGPRPLDQNPG
AGLAAVVASCLSSGFAGVYFEKILKGSSGSVWLRNLQLGLFGTALGLVGL
WWAEGTAVARRGFFFGYTPAVWGVVLNQAFGGLLVAVVVKYADNILKGFA
TSLSIVLSTVASIRLFGFSPGPIICPGRWARHWCRLPLQPSPKCSQSHNF
CLCLCLCLWALHSPAASWAATTTTAAVFSRRPHHGALSAKVAHQGEGFVA
AGIEDTGLPSFSLSCPGPAGTKLLSVLGVG

### Production of Mutations

The mutations in the UDP-galactose transporter (Slc35a2) gene and/or GDP-fucose transporter (Slc35c1) gene may be produced by means known in the art.

Examples of methods that may be used include zinc finger nucleases (ZFNs), transcription activator-like effector nucleases (TALENs), and the RNA-guided CRISPR-Cas nuclease system.

For example, the polynucleotide may be removed from the genome of the cell, using a pair of engineered meganucleases, each of which cleaves a meganuclease recognition site on either side of the intended deletion. TAL Effector Nucleases (TALENs) that are able to recognize and bind to a gene and introduce a double-strand break into the genome may also be used.

TALENs are described in detail in Joung et al (2013) TALENs: a widely applicable technology for targeted genome editing. Nature Reviews Molecular Cell Biology 14, 49-55.

Zinc finger (ZnF) nucleases may also be used for gene editing. Zinc finger nucleases (ZFNs) are synthetic proteins consisting of an engineered zinc finger DNA-binding domain fused to the cleavage domain of a restriction enzyme such as the FokI restriction endonuclease. ZFNs can be used to induce double-stranded breaks in specific DNA sequences and thereby promote site-specific homologous recombination and targeted manipulation of genomic loci in a variety of different cell types.

Zinc finger nucleases TALENs are described in detail in Urnov et al (2010) Genome editing with engineered zinc finger nucleases, Nature Reviews Genetics 11, 636-646.

Methods using the CRISPR/Cas9 system may also be used to engineer edited UDP-galactose transporter (Slc35a2) and GDP-fucose transporter (Slc35c1) genes.

Clustered, regularly interspaced, short palindromic repeat (CRISPR) technology is an important new approach for generating RNA-guided nucleases, such as Cas9, with customizable specificities. Genome editing mediated by these nucleases has been used to rapidly, easily and efficiently modify endogenous genes in a wide variety of biomedically important cell types and in organisms that have traditionally been challenging to manipulate genetically.

Sander et al (2015). CRISPR-Cas systems for editing, regulating and targeting genomes. Nature Biotechnology 32, 347-355.

### Variation and Reproducibility

Homogeneity

Homogeneity may for example be assayed by detecting the number of peaks in a liquid chromatogram of N-glycans of a recombinant polypeptide expressed by a CHO cell comprising reduced UDP-galactose transporter activity, as compared to a control comprising a liquid chromatogram of N-glycans of recombinant polypeptide expressed by wild type CHO-K1.

For example, an increase in homogeneity may be detected as a reduction in the number of peaks as assayed in (a) above, for example to one peak compared to 3 peaks in the control.

Optionally, the number of peaks counted may be such that they are above a pre-determined cut-off of peak height or area under the peak.

Homogeneity may also be assayed by measuring the area under the peak of the major product (e.g., G0F) or products in a liquid chromatogram of N-glycans of a recombinantly expressed polypeptide as a percentage of the total area under the curve.

For example, with reference to Tables D1 to D5 below, the homogeneity for CHO-K1 (wild type) expression of Herceptin is 32.83%, the homogeneity for CHO-gmt2 (lacking functional UDP-galactose transporter) expression of Herceptin is 91.72%, the homogeneity for CHO-gmt3 (lacking functional GDP-fucose transporter) expression of Herceptin is 61.49% and the homogeneity for CHO-gmt9 (lacking functional UDP-galactose transporter and lacking functional GDP-fucose transporter) expression of Herceptin is 90.63%.

Batch-to-Batch Variation

Batch to batch variation may also be assayed by determining the ratio of the respective areas under the peaks of the products in a liquid chromatogram of N-glycans of a recombinantly expressed polypeptide.

An example which refers to Table E2 below follows. Homogeneity or batch-to-batch variation may be determined by comparing columns 1 and 2 of Table E2, which shows the expression of Herceptin in CHO cells lacking functional GDP-fucose transporter (CHO-gmt9 cells, column 1) and expression of Herceptin in parental CHO cells with functional GDP-fucose transporter (column 2). The CHO-gmt3 cell does not make fucosylated glycans.

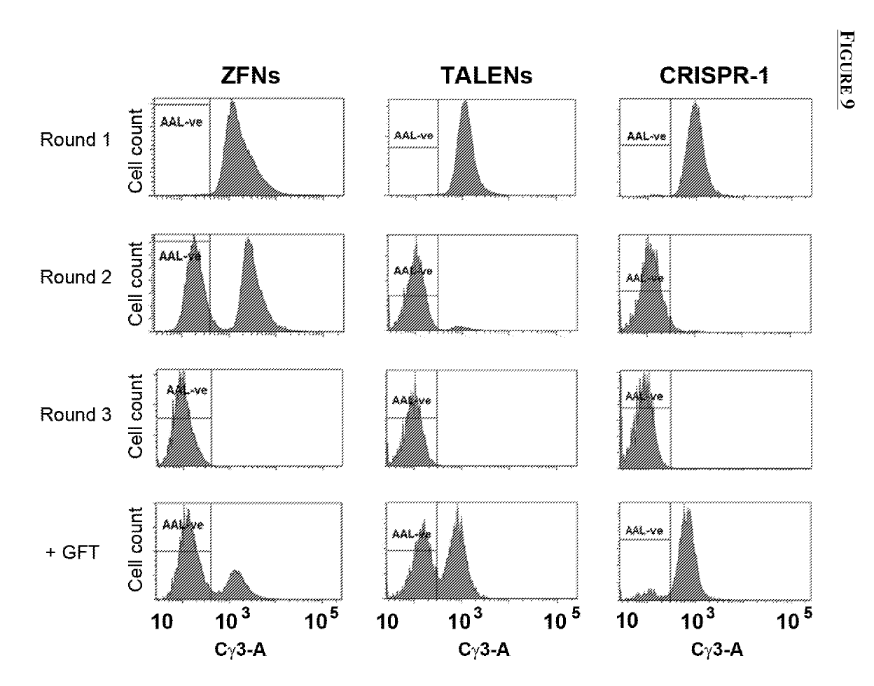

Column 1 (Herceptin) is derived from FIG. 10A and FIG. 10D. Column 2 (Parental CHO-HER) is derived from FIG. 10B and FIG. 10E. Column 3 is derived from FIG. 10C and FIG. 10F.

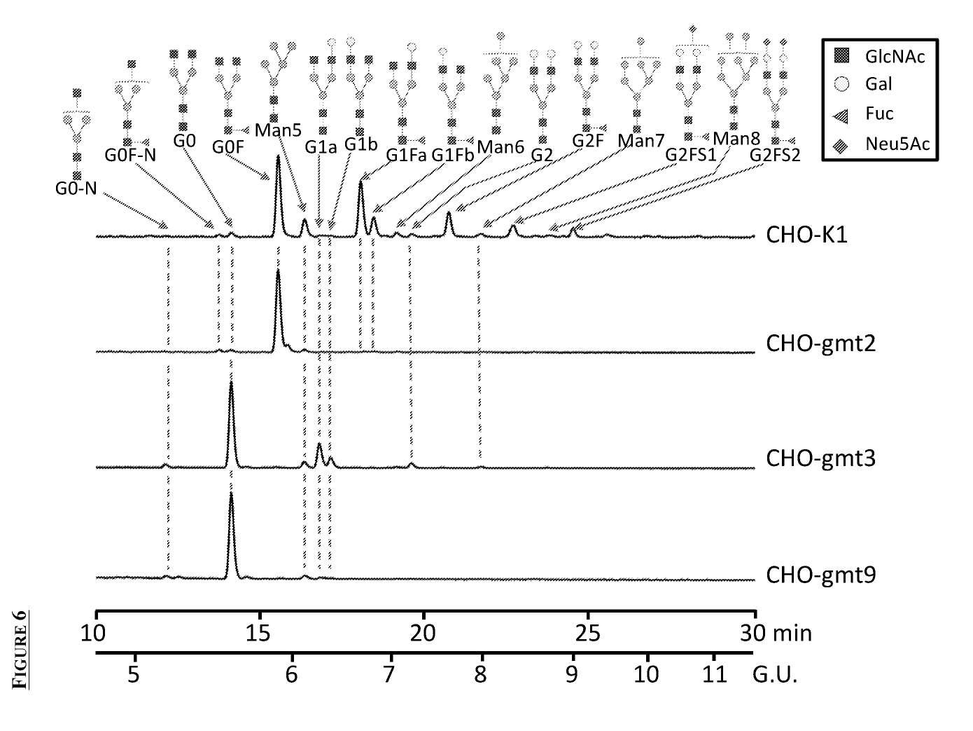

Tables D1 to D5 below show examples of calculation of the areas under the peaks of each of the glycosylation products in a liquid chromatogram of N-glycans (FIG. 6) of a recombinantly expressed polypeptide for the determination of batch-to-batch variation and homogeneity.

TABLE D1
Relative area under each peak of glycosylation products
(rows) for protein expressed from CHO-K1 (“wild type”
CHO cell). A variety of glycosylation types are visible,
with the majority of product being G0F-N.

CHO-K1

| | Retention | | | Relative |
|---|---|---|---|---|
| | Time | Area | Height | Area |
| | min | counts*min | counts | % |
| G0-N | 12.125 | 1420.517 | 8330.952 | 0.24 |
| G0F-N | 13.75 | 6454.601 | 36628.857 | 1.07 |
| G0 | 14.108 | 11256.066 | 60270.429 | 1.87 |
| G0F-N | 15.525 | 197481.833 | 1148438.655 | 32.83 |
| Man5 | 16.325 | 44682.157 | 248842.626 | 7.43 |
| G1a | 16.783 | 3015.558 | 22147.778 | 0.5 |
| G1b | 16.925 | 4994.562 | 17442.283 | 0.83 |
| G1Fa | 18.033 | 131730.334 | 778654.97 | 21.9 |
| G1Fb | 18.425 | 45645.807 | 270582.554 | 7.59 |
| Man6 | 19.117 | 10286.446 | 59131.558 | 1.71 |
| G2 | 19.575 | 7939.608 | 43588.333 | 1.32 |
| G2F | 20.7 | 60690.742 | 348251 | 10.09 |
| Man7 | 21.692 | 11594.275 | 48729.146 | 1.93 |
| G2FS1 | 22.65 | 37105.408 | 169340.214 | 6.17 |
| Man8 | 24.483 | 19078.775 | 126780 | 3.17 |
| G2FS2 | 25.517 | 8060.517 | 37098.652 | 1.34 |

TABLE D2
Relative area under each peak of glycosylation products
(rows) for protein expressed from CHO-gmt2 (lacking
functional UDP-galactose transporter).

CHO-gmt2

| | Retention | | | Relative |
|---|---|---|---|---|
| | Time | Area | Height | Area |
| | min | counts*min | counts | % |
| G0-N | 12.083 | 1783.454 | 9311.415 | 0.77 |
| G0F-N | 13.741 | 5495.359 | 33850.373 | 2.38 |
| G0 | 14.108 | 6271.295 | 29108.173 | 2.71 |
| G0F-N | 15.525 | 212116.32 | 1163575.876 | 91.72 |
| Man5 | 16.325 | 5589.996 | 32663.826 | 2.42 |
| G1a | | | | |
| G1b | | | | |
| G1Fa | | | | |
| G1Fb | | | | |
| Man6 | | | | |
| G2 | | | | |
| G2F | | | | |
| Man7 | | | | |
| G2FS1 | | | | |
| Man8 | | | | |
| G2FS2 | | | | |

TABLE D3
Relative area under each peak of glycosylation products
(rows) for protein expressed from CHO-gmt3 (lacking
functional GDP-fucose transporter).

CHO-gmt3

| | Retention | | | Relative |
|---|---|---|---|---|
| | Time | Area | Height | Area |
| | min | counts*min | counts | % |
| G0-N | 12.117 | 19866.258 | 123903.2 | 2.16 |
| G0F-N | | | | |
| G0 | 14.1 | 565381.011 | 3414064.386 | 61.49 |
| G0F-N | 15.458 | 5126.171 | 27842.375 | 0.56 |
| Man5 | 16.325 | 40564.165 | 233978.605 | 4.41 |
| G1a | 16.775 | 164187.433 | 971145.938 | 17.86 |
| G1b | 17.117 | 71260.569 | 402039.469 | 7.75 |
| G1Fa | | | | |
| G1Fb | | | | |
| Man6 | 19.117 | 12058.933 | 40549.803 | 1.31 |
| G2 | 19.567 | 31224.947 | 180258.895 | 3.4 |
| G2F | | | | |
| Man7 | 21.692 | 9737.942 | 50250.867 | 1.06 |
| G2FS1 | | | | |
| Man8 | | | | |
| G2FS2 | | | | |

TABLE D4
Relative area under each peak of glycosylation products (rows)
for protein expressed from CHO-gmt9 (lacking functional UDP-
galactose transporter and lacking functional GDP-fucose transporter).
The majority of the product is G0 N-glycans.

CHO-gmt9

| | Retention | | | Relative |
|---|---|---|---|---|
| | Time | Area | Height | Area |
| | min | counts*min | counts | % |
| G0-N | 12.125 | 5172.3 | 29027.8 | 2.3 |
| G0F-N | | | | |
| G0 | 14.108 | 204163.428 | 1210454.507 | 90.63 |
| G0F-N | | | | |
| Man5 | 16.342 | 8310.146 | 42835.661 | 3.69 |
| G1a | 16.8 | 4096.618 | 18463.408 | 1.82 |
| G1b | 17.142 | 1485.658 | 9063.854 | 0.66 |
| G1Fa | | | | |
| G1Fb | | | | |
| Man6 | 19.125 | 2031.108 | 9938.132 | 0.9 |
| G2 | | | | |
| G2F | | | | |
| Man7 | | | | |
| G2FS1 | | | | |
| Man8 | | | | |
| G2FS2 | | | | |

TABLE D5
Summary of data showing relative percentages of areas under
peaks of glycosylation products from Tables D1 to D4 above.

| Glycan | Host cell line | | | |
|---|---|---|---|---|
| structure | CHO-K1 | CHO-gmt2 | CHO-gmt3 | CHO-gmt9 |
| G0-N | 0.24 | 0.77 | 2.16 | 2.3 |
| G0F-N | 1.07 | 2.38 | N.D. | N.D. |
| G0 | 1.87 | 2.71 | 61.49 | 90.63 |
| G0F | 32.83 | 91.72 | 0.56 | N.D. |
| Man5 | 7.43 | 2.42 | 4.41 | 3.69 |
| G1a | 0.5 | N.D. | 17.86 | 1.82 |
| G1b | 0.83 | N.D. | 7.75 | 0.66 |
| G1Fa | 21.9 | N.D. | N.D. | N.D. |
| G1Fb | 7.59 | N.D. | N.D. | N.D. |
| Man6 | 1.71 | N.D. | 1.31 | 0.9 |
| G2 | 1.32 | N.D. | 3.4 | N.D. |
| G2F | 10.09 | N.D. | N.D. | N.D. |
| Man7 | 1.93 | N.D. | 1.06 | N.D. |
| G2FS1 | 6.17 | N.D. | N.D. | N.D. |
| Man8 | 3.17 | N.D. | N.D. | N.D. |
| G2FS2 | 1.34 | N.D. | N.D. | N.D. |

Coefficient of Variation

The variability or reproducibility between batches may be expressed in terms of the coefficient of variation.

The coefficient of variation (CV), also known as relative standard deviation (RSD), is a standardized measure of dispersion of a probability distribution or frequency distribution.

It is often expressed as a percentage, and is defined as the ratio of the standard deviation (σ) to the mean (μ) (or its absolute value, |μ|).

cᵥ=σ/μ

As an example, the standard deviation of the areas under peaks of each of the glycosylation products as shown in the above tables may be divided by the mean areas to establish the coefficient of variation.

The coefficient of variation shows the extent of variability in relation to the mean of the population. A high coefficient of variation corresponds to high variability or high batch-to-batch variation and low homogeneity. Conversely, a low coefficient of variation corresponds to low variability or low batch-to-batch variation and high homogeneity.

### Antibody-Dependent Cellular Cytotoxicity (ADCC)

Antibody-dependent cell-mediated cytotoxicity (ADCC) is a mechanism of cell-mediated immune defence whereby an effector cell of the immune system actively lyses a target cell, whose membrane-surface antigens have been bound by specific antibodies. It is one of the mechanisms through which antibodies, as part of the humoral immune response, can act to limit and contain infection.

Classical antibody-dependent cell-mediated cytotoxicity is mediated by natural killer (NK) cells; but macrophages, neutrophils and eosinophils can also mediate it. For example, eosinophils can kill certain parasitic worms known as helminths through ADCC mediated by IgE. ADCC is part of the adaptive immune response due to its dependence on a prior antibody response.

The typical ADCC involves activation of NK cells by antibodies. A NK cell expresses CD16 which is an Fc receptor. This receptor recognizes, and binds to, the Fc portion of an antibody, such as IgG, which has bound to the surface of a pathogen-infected target cell. The most common Fc receptor on the surface of an NK cell is called CD16 or FcγRIII. Once the Fc receptor binds to the Fc region of IgG, the Natural Killer cell releases cytokines such as IFN-γ.

During replication of a virus some of the viral proteins are expressed on the cell surface membrane of the infected cell. Antibodies can then bind to these viral proteins. Next, the NK cells which have Fc Receptors will bind to that antibody, inducing the NK cell to release proteins such as perforin and proteases known as granzymes, which causes the lysis of the infected cell to hinder the spread of the virus.

Furthermore, NK cells are involved in killing tumor cells and other cells that may lack MHC I on their surface, indicating a non-self cell. This is because, generally, all nucleated cells (which excludes RBCs) of the body contain MHC I.

ADCC by Eosinophils

Large parasites like helminths are too big to be engulfed and killed by phagocytosis. They also have an external structure or integument that is resistant to attack by substances released by neutrophils and macrophages. After IgE coat these parasites, the Fc receptor (FceRI) of an eosinophil will then recognize IgE. Subsequently, interaction between FceRI and the Fc portion of helminth-bound IgE signals the eosinophil to degranulate.

ADCC In Vitro

Several laboratory methods exist for determining the efficacy of antibodies or effector cells in eliciting ADCC. Among these methods include chromium-51 [Cr51] release assay, europium [Eu] release assay, and sulfur-35 [S35] release assay. Usually, a labelled target cell line expressing a certain surface-exposed antigen is incubated with antibody specific for that antigen. After washing, effector cells expressing Fc receptor CD16 are co-incubated with the antibody-labelled target cells. Target cell lysis is subsequently measured by release of intracellular label by a scintillation counter or spectrophotometry.

A common challenge faced by ADCC assays is high background signaling due to cellular “leakiness”. While both Cr51 and Eu-based assays face this challenge, S35-containing methionine and cysteine pre-incubated with target cells leads to incorporation of radio-labelled molecules into newly translated peptides.

The coupled bioluminescent method aCella TOX is now in widespread use for ADCC and other cytotoxicity assessments. Since this technique measures the release of enzymes naturally present in the target cells, no labelling step is required and no radioactive agents are used.

[The text in this section is adapted from Antibody-dependent cell-mediated cytotoxicity. (2015, Jul. 17). In Wikipedia, The Free Encyclopedia. Retrieved 09:11, Oct. 9, 2015, from https://en.wikipedia.org/w/index.php?title=Antibody-dependent_cell-mediated_cytotoxicity&oldid=671894320]

### CHO Cells and Cell Lines

The CHO cells and cell lines described here may be made by any suitable means.

As another example, the CHO cells and cell lines may be produced by gene engineering or gene editing, using methods such as TALENs, zinc finger nucleases or CRISPR/Cas 9, as described elsewhere in this document.

As a specific example, which is not intended to be limiting, one or more genes in CHO-K1 cells may be inactivated by zinc-finger nuclease (ZFN) technology. The method used may be as described in Zhang et al. 2012.

Mutants with the relevant gene interrupted by the ZFNs may be identified by sequencing the targeted locus and confirmed by the genetic complementation test with functional cDNA (Zhang et al., 2012).

The CHO cells and cell lines may also be produced by selection using a suitable agglutinating agent, such as agglutinin. The agglutinin may comprise any suitable agglutinin, such as Maackia amurensis agglutinin (MAA).

We therefore provide a method of providing a CHO cell or cell line, the method comprising culturing CHO cells in the presence of Maackia amurensis agglutinin (MAA) and selecting cells which survive the culture.

The CHO cells and cell lines described here may be made by treating a starting or parent cell with Maackia amurensis agglutinin (MAA) and selecting cells that survive such treatment. Such surviving cells may be further cloned and made into cell lines. The selected cells and cell lines may be selected for any desired characteristics as described in this document. The selected cells and cell lines may comprise mutant UDP-galactose Transporter (Slc35a2) or GDP-fucose Transporter (Slc35c1) genes and polypeptides, as described in this document.

For example, the CHO cells or cell lines described here may be selected by exposing a parent cell line to Maackia amurensis agglutinin (MAA) at a suitable concentration for a suitable period.

The Maackia amurensis agglutinin (MAA) could range from between 0.1 μg/ml to 100 μg/ml, for example up to 50 μg/ml or up to 20 μg/ml. Examples of specific concentrations include 10 μg/ml and 5 μg/ml.

The period of incubation or exposure to Maackia amurensis agglutinin (MAA) could be from an hour, a few hours (such as 2, 3, 4, 5, 6, 7, 8, 9, 10, 11, 12), overnight, to a few days, such as 2 days or 3 days.

In general, the period and concentration can be adjusted to eliminate the majority of CHO cells, but to enable a small proportion of cells, which are resistant to Maackia amurensis agglutinin (MAA), to survive and form colonies. Within this, the concentration and period of exposure and selection may be varied, but generally, the higher the concentration of Maackia amurensis agglutinin (MAA), the lower the period of exposure is necessary, and vice versa.

The selection could be done on any suitable starting cell or cell line, but this will generally be a CHO cell or cell line. Any known CHO cell or cell line could be used as a starting point or parent cell, including CHO-K1. Other suitable starting cells could include, but are not limited to the following (ECACC accession numbers in brackets): CHO (85050302), CHO (PROTEIN FREE) (00102307), CHO-K1 (85051005), CHO-K1/SF (93061607), CHO/dhFr- (94060607), CHO/dhFr-AC-free (05011002), RR-CHOKI (92052129).

Following selection, the surviving cells are allowed to grow and form colonies following which they may be picked. The time allowed for this will vary, but will generally be long enough for colonies to grow to a pickable size. Examples of such times are 5 days, 7 days, 9 days, 11 days, 13 days, one week, two weeks, three weeks or more.

The picking may be done manually, or it may be automated through use of robots, such as CLONEPIX (Genetix, New Milton, Hampshire, UK). The picked colonies may be further cloned, further screened, characterised and cultured, etc.

The selected cells may be subjected to further tests. For example, they may be subjected to agglutination tests using Maackia amurensis agglutinin (MAA) to confirm the mutant cells no longer react with Maackia amurensis agglutinin (MAA).

As a specific example, which is not intended to be limiting, CHO-K1 cells may be seeded into 10-cm culture dishes and cultured overnight. On the following day, the culture medium may be replaced with serum-free DMEM containing 50 μg/ml of Maackia amurensis agglutinin (MAA) for 12 h, as previously described (Lim et al., 2008).

The cells may then be cultured until surviving cells formed individual colonies. MAA-resistant clones may be isolated and characterized for deficiency in UDP-galactose transporter (Slc35a2) activity by the EPO rescue assay (Lim et al., 2008) by co-transfecting the cells with constructs expressing EPO and functional human UDP-galactose transporter.

These cells may be subjected to further tests, such as agglutination tests using Maackia amurensis agglutinin (MAA) to confirm the mutant cells no longer react with Maackia amurensis agglutinin (MAA). We therefore provide for a method of providing a CHO cell or cell line, the method comprising culturing CHO cells in the presence of Maackia amurensis agglutinin (MAA), selecting cells which survive the culture and which do not react with Maackia amurensis agglutinin (MAA) in an agglutination test.

The Maackia amurensis agglutinin (MAA) selected CHO cells and CHO cell lines may be tested for a desired behaviour, by for example expressing a protein of interest and determining the degree of batch-to-batch variation, homogeneity, binding to FcγRIII (CD16), recruitment of effector cells such as natural killer (NK) cells or antibody-dependent cellular cytotoxicity (ADCC), or any combination thereof.

We therefore provide for a method of providing a CHO cell or cell line, the method comprising culturing CHO cells in the presence of Maackia amurensis agglutinin (MAA), selecting cells which survive the culture and selecting those cells or cell lines which display high desired behaviour behaviour, as described above.

The UDP-galactose Transporter (Slc35a2) or GDP-fucose Transporter (Slc35c1) gene in such selected cells may be cloned and sequenced, using methods known in the art. The UDP-galactose Transporter (Slc35a2) or GDP-fucose Transporter (Slc35c1) gene may comprise a mutant UDP-galactose Transporter (Slc35a2) gene or a mutant GDP-fucose Transporter (Slc35c1) gene as described here.

We therefore provide for a method of providing a CHO cell or cell line, the method comprising culturing CHO cells in the presence of Maackia amurensis agglutinin (MAA), selecting cells which survive the culture and selecting those cells or cell lines which comprise mutant UDP-galactose Transporter (Slc35a2) or GDP-fucose Transporter (Slc35c1) genes as described herein.

### Mutant CHO Cells and Cell Lines

We provide for a CHO cell or cell line derived from Maackia amurensis agglutinin (MAA) selection, as described above. Such a cell line could include a CHO-gmt2 cell line, a CHO-gmt3 cell line or a CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

### Protein Expression

The CHO cells described here may be used as host cells for expression of any protein of interest. This may be done by means known in the art.

Protein expression in CHO cells and cell lines is well described in the literature, and the skilled person will have little difficulty in using the CHO cells and cell lines described here as hosts for protein expression. Thus, for example, the CHO cells and cell lines may be transfected by means known in the art with expression vectors capable of expressing the protein of interest.

Any suitable protein may be expressed using the CHO cells described here as host cells. The protein may comprise a heterologous protein. The protein may comprise a recombinant protein. The protein may comprise an engineered protein. The protein may comprise a glycoprotein.

Examples include heterologous proteins of therapeutic or pharmacological interest. Proteins which may be expressed include anti-EGFR mAb, α-glucosidase, laronidase, Ig-CTLA4 fusion, N-acetylgalactosamine-4-sulfatase, luteinizing hormone, anti-VEGF mAb, Factor VIII, anti-lgE mAb, anti-CD11a mAb, α-galactosidase, interferon-β, anti-TNFα mAb, erythropoietin, anti-CD52 mAb, Factor VIII, tissue plasminogen activator, anti-HER2 mAb, TNFα receptor fusion, Factor IX, follicle stimulating hormone, anti-CD20 mAb, interferon-β, β-glucocerebrosidase, deoxyribonuclease I, etc.

For example, we describe the expression of erythropoietin (EPO), erythropoietin-Fc fusion polypeptide (EPO-Fc), MUC1-Fc fusion polypeptide, an antibody, anti-HER2 antibody (Herceptin), Anti-CD20 antibody (Rituxan or GA101), IgG1, IgG2, IgG3 or IgG4 with CHO cells and cell lines described here.

### Recombinant Polypeptides

The methods and compositions described here may used for the expression of any polypeptide of interest. The expressed polypeptide may comprise a recombinant polypeptide.

The expressed polypeptide may comprise any glycopeptide. The expressed polypeptide may comprise a biologic or a biosimilar.

The expressed polypeptide may comprise an antibody. The expressed polypeptide may comprise a monoclonal antibody. The expressed polypeptide may comprise a monoclonal antibody (mAb) involved in antibody-dependent cellular cytotoxicity (ADCC). The expressed polypeptide may comprise an IgG molecule.

A number of IgGs produced by recombinant DNA technology are currently being marketed as human therapeutics to treat life-threatening diseases. Additionally, a number of other IgGs are in various phases of human clinical trials for development as human therapeutics.

Any one or more of these antibodies or IgGs may suitably be produced using the methods and compositions described here. Examples of such antibodies are provided in the publications of Scott et al (2012). Antibody therapy of cancer. Nat Rev Cancer. 12(4):278-87 and Niwa et al (2014). Glyco-engineered Therapeutic Antibodies as a Second-Generation Antibody Therapy. Glycoscience: Biology and Medicine pp 1-8.

As examples, the following antibody polypeptides may be expressed Trastuzumab (Herceptin; Genentech): humanized IgG1; Bevacizumab (Avastin; Genentech/Roche): humanized IgG1; Cetuximab (Erbitux; Bristol-Myers Squibb): chimeric human-murine IgG1; Panitumumab (Vectibix; Amgen): human IgG2; Ipilimumab (Yervoy; Bristol-Myers Squibb): IgG1; Rituximab (Mabthera; Roche): chimeric human-murine IgG1; Alemtuzumab (Campath; Genzyme): humanized IgG1; Ofatumumab (Arzerra; Genmab): human IgG1; Gemtuzumab ozogamicin (Mylotarg; Wyeth): humanized IgG4; Brentuximab vedotin (Adcetris; Seattle Genetics): chimeric IgG1; ⁹⁰Y-labelled ibritumomab tiuxetan (Zevalin; IDEC Pharmaceuticals): murine IgG1; ¹³¹I-labelled tositumomab (Bexxar; GlaxoSmithKline): murine IgG2; Mogamulizumab/POTELIGEO®/KW-0761 (Kyowa Hakko Kirin/CCR4); Obinutuzumab/GA101 (Roche/GlycArt/CD20); Benralizumab/MEDI-563 (AstraZeneca/MedImmune/IL-5Rα/phase III); MEDI-551 (AstraZeneca/MedImmune/CD19/phase II); Ecromeximab/KW-2871 (Life Science Pharmaceuticals/GD3/phase II); Roledumab/LFB-r593 (LFB/RhD/phase II); GA201 (Roche/GlycArt/EGFR/phase II); ARGX-110 (arGEN-X/CD70/phase II); BIW-8962 (Kyowa Hakko Kirin/GM2/phase I) or KHK2898 (Kyowa Hakko Kirin/CD98/phase I).

The expressed polypeptide may comprise Orthoclone OKT3 (Johnson & Johnson), which was approved June 1986 for allograft rejection.

The expressed polypeptide may comprise ReoPro (Lilly), which was approved December 1994 for PTCA adjunct. The expressed polypeptide may comprise Rituxan (Genentech), which was approved November 1997 for non-Hodgkin's lymphoma.

The expressed polypeptide may comprise Simulect (Novartis), which was approved May 1998 for organ rejection prophylaxis. The expressed polypeptide may comprise Remicade (Johnson & Johnson), which was approved August 1998 for heumatoid arthritis was Crohn's disease. The expressed polypeptide may comprise Zenapax (Roche), which was approved December 1997 for organ rejection prophylaxis.

The expressed polypeptide may comprise Synagis (Medimmune), which was approved June 1998 for respiratory syncytial virus (RSV). The expressed polypeptide may comprise Herceptin (Genentech), which was approved September 1998 for metastatic breast cancer.

The expressed polypeptide may comprise Mylotarg (American Home Products), was approved May 2000 for acute myeloid leukemia. The expressed polypeptide may comprise Campath (Millennium), which was approved July 2001 for chronic lymphocytic leukemia.

Humira (Abbott Laboratories), which was approved December 2002 for disease modifying antirheumatic drugs (DMARDs).

### EXAMPLES

### Example 1

### Materials and Methods: Isolation of UDP-Galactose Transporter (Slc35a2)-Deficient CHO Mutants, CHO-gmt2 Cells

CHO-K1 cells were purchased from ATCC or obtained originally from Dr. Donald K. MacCallum (University of Michigan Medical School, Ann Arbor, Mich.).

The cells were cultured in Dulbecco's Modified Eagle's Medium (DMEM, Life Technologies, Carlsbad, Calif.) supplemented with 10% New Zealand fetal bovine serum (FBS, Life Technologies, Carlsbad, Calif.) at 37° C. with 5% CO₂.

The cells were seeded into 10-cm culture dishes and cultured overnight. On the following day, the culture medium was replaced with serum-free DMEM containing 50 μg/ml of Maackia amurensis agglutinin (MAA) for 12 h, as previously described (Lim et al., 2008).

The cells were then cultured until surviving cells formed individual colonies. MAA-resistant clones were isolated and characterized for deficiency in UDP-galactose transporter (Slc35a2) activity by the EPO rescue assay (Lim et al., 2008) by co-transfecting the cells with constructs expressing EPO and functional human UDP-galactose transporter.

Total RNA was extracted from the UDP-galactose transporter-rescued clones using RNeasy mini kit (Qiagen, Valencia, Calif.) and cDNA was synthesized using SuperScript® III Reverse Transcriptase (Life Technologies, Carlsbad, Calif.), according to the manufacturers' protocol.

PCR amplification of the Slc35a2 locus was carried out and the amplicon sequenced to characterize the mutation. The CHO cells that carry dysfunctional UDP-galactose transporter gene are called CHO-gmt2 cells.

### Example 2

### Materials and Methods: Isolation of GDP-Fucose Transporter (Slc35c1) Deficient CHO Mutants, CHO-gmt3 Cells

The gene encoding the GDP-fucose transporter (Slc35c1) in CHO cells was inactivated by zinc-finger nuclease (ZFN) technology by following a previous publication (Zhang et al. 2012).

The mutants with their GDP-fucose transporter gene interrupted by the ZFNs were identified by sequencing the targeted locus and confirmed by the genetic complementation test with functional GDP-fucose transporter cDNA (Zhang et al., 2012).

The CHO cells that carry dysfunctional GDP-fucose transporter gene are called CHO-gmt3 cells.

### Example 3

### Materials and Methods: Isolation and Characterization of Slc35a2 and Slc35c1 Double Deficient CHO Mutants, CHO-gmt9 Cells

CHO-gmt2 cells that lack functional UDP-galactose transporter (Slc35a2) were seeded into each well of a 6-well plate at a density of 6×10⁵ cells 24 h prior to transfection.

The following day, plasmid constructs expressing the pair of ZFNs targeting the gene Slc35c1 were transiently transfected using Lipofectamine 2000 as previously described (Zhang et al., 2012).

The medium was replaced 6 h after transfection and the cells were cultured for another 48-72 h before they were subjected to staining with biotinylated AAL lectin and streptavidin-conjugated Cy3 dye and sorted for negatively stained cells.

Approximately 500-1000 AAL-negative cells were cultured in 15-cm culture dishes until individual colonies were formed. Single clones were isolated and characterized for deficiency in the gene Slc35c1 by AAL-Cy3 staining assay.

Genetic characterization of the mutation in Slc35c1 was carried out by PCR amplifying and sequencing the genomic DNA region that was targeted by the ZFNs, as previously described (Zhang et al., 2012).

### Example 4

### Materials and Methods: Transient Expression and Isoelectric Focusing Analysis of EPO

The various CHO cell lines were seeded into 6-well plates at a density of 6×10⁵ cells per well.

On the following day, CHO-K1 was transiently transfected with EPO-expressing construct, while CHO-gmt2, CHO-gmt3, and CHO-gmt9 were transfected with either EPO only or EPO- and a UDP-galactose transporter-expressing construct.

Forty eight hours after transfection, the conditioned media were collected for analysis using IEF, pH range 3 to 10, and Western blot using previously published methods (Schriebl et al., 2006).

Prior to loading, the samples were desalted against 20 mM phosphate buffer in an Amicon® Ultra-4 centrifugal filter units with 10-kDa cutoff membrane (Millipore, Billerica, Mass.).

### Example 5

### Materials and Methods: FACS Analysis of AAL-Stained CHO-K1, CHO-gmt2, CHO-gmt3, and CHO-gmt9 Cells

CHO-K1, CHO-gmt2, CHO-gmt3, and CHO-gmt9 cells were seeded into 6-well plates ata density of 6×10⁵ cells per well.

The following day, either empty vector or GDP-fucose transporter-expressing construct was transiently transfected.

Forty eight hours after transfection, the cells were collected, stained with biotinylated AAL and streptavidin-Cy3, and sorted on a BD FACSAria III cell sorter (BD Biosciences, San Jose, Calif.).

### Example 6

### Materials and Methods: Transient Expression of EPO-Fc and MUC1-Fc in Different CHO Cell Lines for N- and O-Glycan Analyses

Fc fusion protein of human erythropoietin (EPO-Fc) and human mucin-1 (MUC1-Fc) were produced in CHO-K1, CHO-gmt2, CHO-gmt3 and CHO-gmt9 cells as described previously (Goh et al., 2010) for N-glycan structure analyses.

Fc region from human IgG1 was fused to the C-terminus of EPO by overlap PCR. The PCR product for the EPO-Fc fusion was cloned into the vector pcDNA3.1 (Life Technologies, Carlsbad, Calif.) with a Kozak sequence placed upstream of the translation start codon ATG.

Similarly, the coding region for the extracellular N-terminal domain of human MUC1 with 5 tandem repeats of the amino acid residues—HGVTSAPDTRPAPGSTAPPA—was fused to the Fc region from human IgG1 by overlap PCR and cloned into pcDNA3.1 as previously described (Lim et al., 2008).

For each cell line, 1.4×10⁷ cells were seeded into each of ten T-175 flasks and cultured overnight.

The following day, construct expressing either EPO-Fc or MUC1-Fc was transiently transfected. Six hours after transfection, the cells were rinsed with DPBS and the medium replaced with chemically defined serum-free medium, comprising 50% (v/v) HyClone™ PF-CHO™ (Thermo Scientific, Waltham, Mass.) with 2 g/l sodium bicarbonate, and 50% (v/v) 1× CD CHO Medium (Life Technologies, Carlsbad, Calif.), supplemented with 6 mM L-glutamine (Life Technologies, Carlsbad, Calif.) and 0.5× Pluronic® F-68 (Life Technologies, Carlsbad, Calif.).

The conditioned media containing the secreted recombinant protein were then collected every 2-3 days over the course of 7 days, and run through a HiTrap Protein A HP column and purified using an FPLC AKTA Purifier (GE Healthcare, Pittsburgh, Pa.) for glycan analyses.

### Example 7

### Materials and Methods: Transient Expression of Trastuzumab (Herceptin) for N-Glycan Analysis

Herceptin light chain and heavy chain with optimized signal peptides were fused with an internal ribosome entry site (IRES) in between, and cloned into pcDNA3.1.

Large scale transient transfection into CHO-K1, CHO-gmt2, CHO-gmt3, and CHO-gmt9 cells, and collection of the conditioned media were carried out as described above.

The recombinant antibody produced by different CHO cells was purified by a HiTrap Protein A HP column using the FPLC AKTA Purifier for glycan analyses

### Example 8

### Materials and Methods: Analysis of the Oligosaccharides Released from Recombinant Proteins using MALDI-TOF MS

An amount of 200 μg of purified EPO-Fc or MUC1-Fc was used for carbohydrate structure analyses.

The carbohydrates liberated by PNGase F were analyzed by matrix-assisted laser desorption/ionization time-of-flight mass spectrometry (MALDI-TOF MS) and HILIC-UPLC, as previously described (Ho et al., 2012; Loh et al., 2014).

### Example 9

### Materials and Methods: Growth Curves of CHO-K1, CHO-gmt2, CHO-gmt3, and CHO-gmt9 Cells

Adherent CHO-K1, CHO-gmt2, CHO-gmt3, and CHO-gmt9 were gradually adapted to suspension culture in chemically defined serum-free medium as described above.

Each cell line was seeded at a cell density of 2.5×10⁵ cells per ml in a 25-ml suspension culture in triplicates. Cell density and viability were measured at 24 h intervals until cell viability fell below 50% and growth curve was generated.

### Example 10

### Results: Isolation and Characterization of UDP-Galactose Transporter (Slc35a2)-Deficient CHO-Mutants, CHO-gmt2 Cells

Maackia amurensis agglutinin (MAA) is a plant lectin that specifically recognizes α2,3-linked sialic acid to galactose residues (Knibbs et al., 1991; Wang and Cummings, 1988). CHO cells are known to exclusively express sialic acid-α2,3-galactose epitopes, not the sialic acid-α2,6-galactose epitopes (Conradt et al., 1987).

MAA is highly cytotoxic towards CHO cells and has been used to isolate glycosylation mutants from wild-type CHO cells (Lim et al., 2008). Following an overnight treatment of CHO-K1 cells with MAA at a concentration of 50 μg/ml, all the wild-type CHO cells were killed possibly by apoptosis. A panel of MAA-resistant clones that survived the MAA treatment was isolated two weeks later.

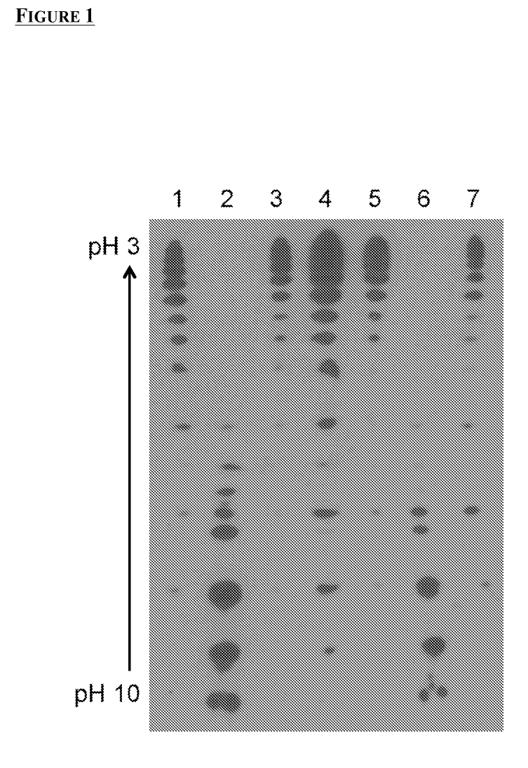

These CHO glycosylation mutants were analyzed by a genetic complementation test using the EPO/IEF assay (Lim et al., 2008). Recombinant EPO transiently expressed in one of the UDP-galactose transporter (Slc35a2)-deficient clones has a clear defect in glycosylation as the bands representing the different glycoforms of EPO are clustered towards the basic end of the gel as shown in lane 2 of FIG. 1.

The glycosylation defect in this mutant was rescued by co-transfecting the mutant cells with a construct encoding UDP-galactose transporter (Slc35a2) gene as shown in lane 3 of FIG. 1. This confirms that the phenotypic lack of normal glycosylation in this clone was due to a defect in the Slc35a2 gene.

All the mutant clones that carry dysfunctional UDP-galactose transporter Slc35a2 gene have been assigned the name CHO-gmt2.

The total mRNA was isolated from this mutant clone and the cDNA encoding the UDP-galactose transporter was amplified by PCR and sequenced.

A single T insertion was identified at the nucleotide position 955 of the Slc35a2 open reading frame. This mutation results in a frame shift starting at amino acid 319. Compared to the normal UDP-galactose transporter which contains 398 amino acids, the C-terminal 79 amino acids in the mutant were changed to different amino acids.

### Example 11

### Results: Isolation and Characterization of GDP-Fucose Transporter (Slc35c1)-Deficient CHO-Mutants, CHO-gmt3 Cells

CHO mutants that lack functional GDP-fucose transporter (Slc35c1) gene are assigned the name CHO-gmt3.

We first inactivated the GDP-fucose transporter (Slc35c1) gene in CHO cells by zinc finger nucleases (ZFNs) (Zhang et al., 2012). Recently, we knocked out the same gene in CHO cells by TALENs.

The CHO-gmt3 line used in this study was generated by ZFNs and it has a 2-nucleotide deletion at 412-413 positions of the open reading frame in one allele and a 35-nucleotide deletion from 402 to 436 in another allele. The UDP-galactose transporter (Slc35a2) gene in CHO-gmt3 cells is normal.

As shown in FIG. 1, the sialylation patterns of the recombinant EPO produced by CHO-gmt3 cells are similar to that produced by the wild-type CHO-K1 cells (FIG. 1, lane 4) and are not changed by co-expressing UDP-galactose transporter (Slc35a2) (FIG. 1, lane 5).

### Example 12

### Results: Isolation and Characterization of UDP-Galactose Transporter (Slc35a2)-Deficient and GDP-Fucose Transporter (Slc35c1)-Deficient CHO-Mutants, CHO-gmt9 Cells

The GDP-fucose transporter (Slc35c1) gene was inactivated by ZFNs as described by Zhang et al. (2012) in the UDP-galactose transporter (Slc35a2)-deficient CHO-gmt2 line.

The CHO cells that are deficient in both GDP-fucose transporter (Slc35c1) and the UDP-galactose transporter (Slc35a2) are named CHO-gmt9.

The CHO-gmt9 line used in this study carries the same single T insertion at the nucleotide position 955 of the Slc35a2 open reading frame as in the CHO-gmt2 line used in this study. The GDP-fucose transporter (Slc35c1) gene in this CHO-gmt9 line has a 3-nucleotide (GTA) insertion at position 411 of the open reading frame in one of its alleles and a 4-nucleotide insertion at position 411 of another allele. Both insertions result in a stop codon mutation.

Due to the lack of functional UDP-galactose transporter (Slc35a2) gene in CHO-gmt9 cells, recombinant EPO produced by CHO-gmt9 cells was not properly sialylated, similar to that produced by CHO-gmt2 cells (FIG. 1, lane 6). The EPO was sialylated similar to that produced by the wild-type cells when the UDP-galactose transporter was co-expressed in the CHO-gmt9 cells (FIG. 1, lane 7).

### Example 13

### Results: Expression of GDP-Fucose Transporter Restored Fucosylation Capabilities in CHO-gmt3 and CHO-gmt9 Cells

Aleuria aurantia lectin (AAL) is one of the lectins that recognize the core fucose on the N-glycans (Matsumura et al., 2007).

We have developed an AAL staining combined with fluorescence activated cell sorting (FACS) method to analyze the expression of core fucose on the surface of CHO cells.

We have shown that CHO-gmt5 cells fail to react with AAL due to the lack of functional GDP-fucose transporter. Expression of GDP-fucose transporter in CHO-gmt5 cells was able to change them to AAL-positive cells (Haryadi et al., 2013; Zhang et al., 2012).

In order to confirm the lack of core fucosylation due to defective SLC35C1, GDP-fucose transporter rescue assay was carried out, followed by FACS analysis of the transfectants.

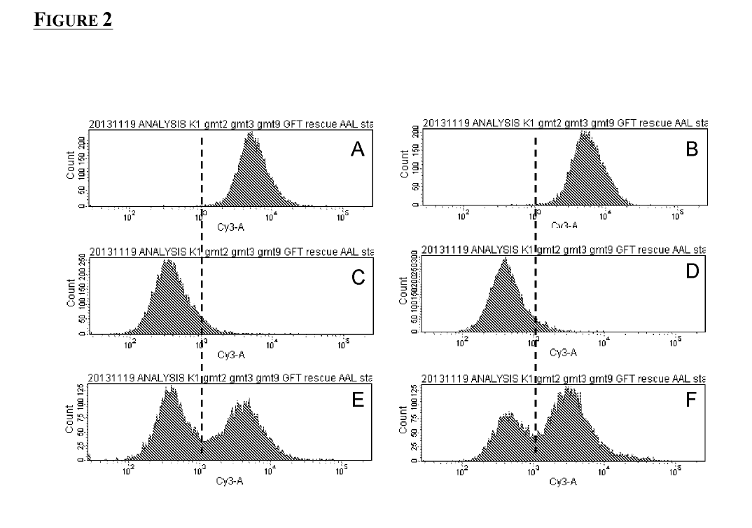

As shown in FIG. 2, wild-type CHO cells (FIG. 2, A) and CHO-gmt2 cells (FIG. 2, B) react strongly with AAL. CHO-gmt3 (FIG. 2, C) and CHO-gmt9 (FIG. 2, D) cells failed to react with AAL. However, after transfection with a construct that expresses GDP-fucose transporter, a portion of CHO-gmt3 and CHO-gmt9 become AAL-positive (FIG. 2, E and FIG. 2, F, respectively), confirming that both cell lines lack functional GDP-fucose transporter.

Taken together, we have confirmed that CHO-gmt2 cells lack functional UDP-galactose transporter and CHO-gmt3 lacks GDP-fucose transporter activity, whereas CHO-gmt9 cells lack both activities.

### Example 14

### Results: N-Glycan Structure Analysis of Recombinant EPO-Fc Produced in CHO-K1, CHO-gmt2, CHO-gmt3, and CHO-gmt9 Cells

In order to elucidate the structural differences of the N-glycans in our mutant CHO cell lines compared with wild-type CHO-K1, recombinant EPO-Fc was used as model molecule.

Following large scale transient transfection of EPO-Fc construct, the conditioned medium from each cell line was collected over 7 days and purified through protein A column and analyzed using MALDI-TOF-MS.

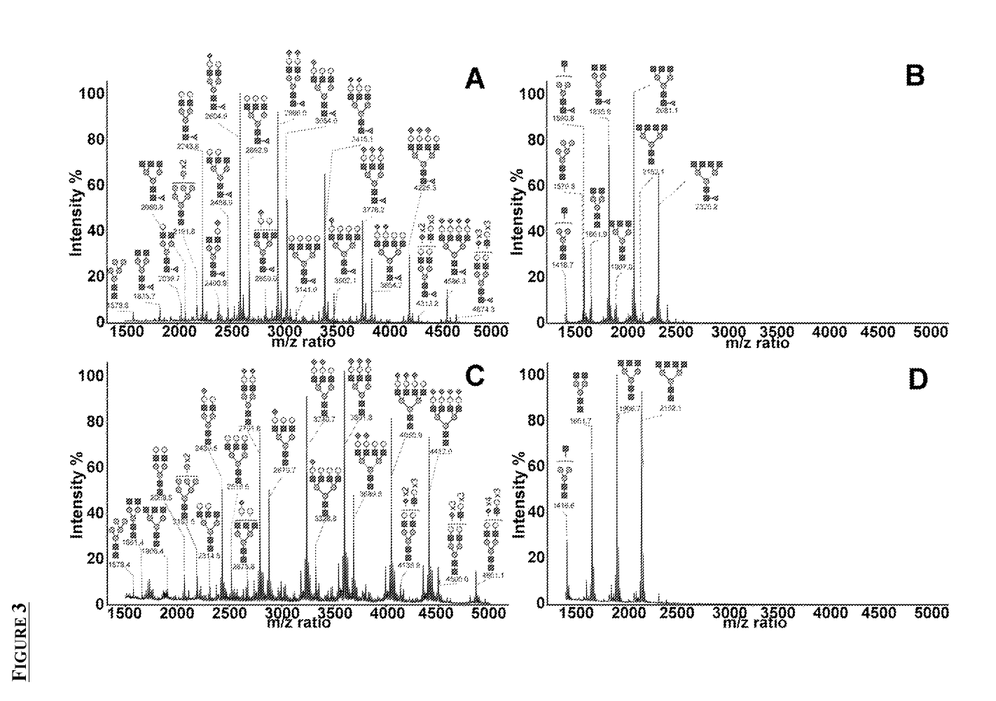

FIG. 3 shows the N-glycan profiles of EPO-Fc as produced in (A) CHO-K1, (B) CHO-gmt2, (C) CHO-gmt3, and (D) CHO-gmt9. Compared to CHO-K1, the N-glycans produced by CHO-gmt2 and CHO-gmt9 cells lack terminal sialic acid and galactose residues, hence terminating with N-acetylglucosamine (GlcNAc) residues.

Furthermore, unlike CHO-K1, the N-glycans in CHO-gmt3 and CHO-gmt9 cells lack core fucose.

### Example 15

### Results: O-Glycan Structure Analysis of Recombinant MUC1-Fc Produced in CHO-K1, CHO-gmt2, CHO-gmt3, and CHO-gmt9

In order to elucidate the structural differences of the N-glycans in our mutant CHO cell lines compared with wildtype CHO-K1, recombinant human MUCl-Fc was used as model molecule.

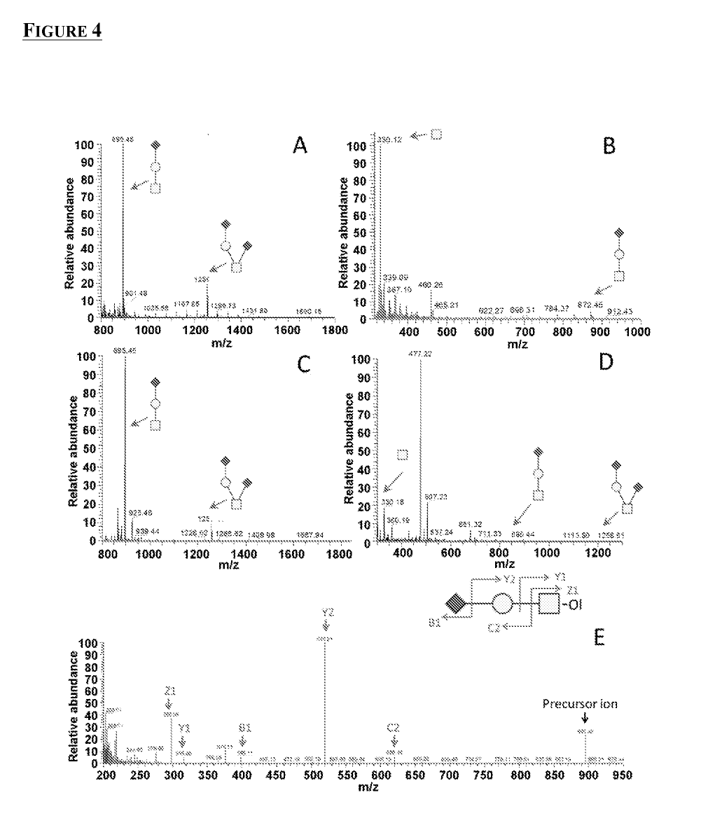

Similar to the EPO-Fc transient transfection described above, the conditioned medium from each cell line containing MUCl-Fc was collected over 7 days and purified through protein A column and analyzed using MALDI-TOF-MS. FIG. 4 shows the O-glycan profiles of MUC1-Fc as produced in (A) CHO-K1, (B) CHO-gmt2, (C) CHO-gmt3, and (D) CHO-gmt9. Compared with CHO-K1 and CHO-gmt3, the O-glycans in CHO-gmt2 and CHO-gmt9 terminate with either N-acetylgalactosamine (GalNAc) residue (Tn antigen), or with sialic acid a 2,6-linked to the GalNAc residue (sTn antigen).

This is because the lack of functional SLC35A2 prevents addition of galactose residue to GalNAc to form the Core 1-O-glycan (T antigen).

### Example 16

### Results: N-Glycans of Released from Trastuzumab (Herceptin) Produced in CHO-K1, CHO-gmt2, CHO-gmt3, and CHO-gmt9 Cells

Lastly, using Herceptin as model antibody, we aim to carry out a glycomics analysis of antibody glycosylation in the three mutant CHO cell lines, as compared with the wildtype CHO-K1.

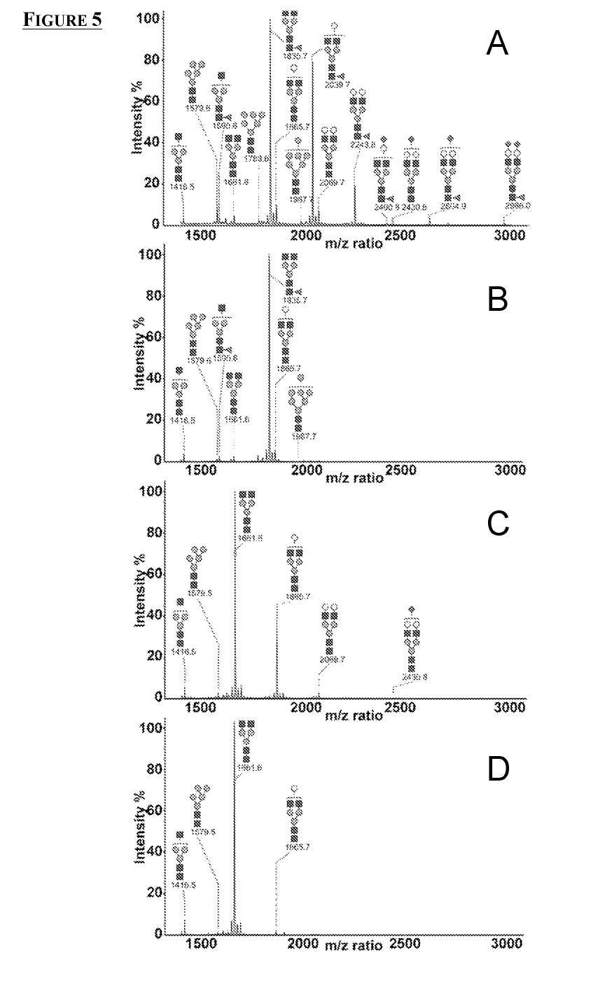

It is known that N-glycosylation of IgG at amino acid residue Asn²⁹⁷ in its Fc region is important for its functional properties. Following large scale transient transfection and purification, the samples were analyzed using MALDI-TOF-MS and UHPLC. FIG. 5 shows the carbohydrate profiles of Herceptin as produced in (A) CHO-K1, (B) CHO-gmt2, (C) CHO-gmt3, and (D) CHO-gmt9.

Consistent with the aforementioned N-glycan analysis carried out on EPO-Fc, the biantennary N-glycans of Herceptin produced in CHO-gmt2 lack sialic acid and galactose residues, forming what is termed as IgG-G0 glycovariant.

Similarly, the N-glycans of Herceptin produced in CHO-gmt3 lack core fucose residue, forming IgG-F0. CHO-gmt9, which lacks both SLC35A2 and SLC35C1, produces Herceptin that lacks sialic acid and galactose residues, as well as the core fucose, and this is termed IgG-G0F0.

Similar patterns were observed using UPLC as shown in FIG. 6.

### Example 17

### Results: Adaptation of CHO-K1, CHO-gmt2, CHO-gmt3, and CHO-gmt9 Cells to Suspension Culture in Serum-Free Medium

Our panel of mutant CHO cell lines and wild-type CHO-K1 were subsequently adapted to grow in suspension culture in chemically defined, serum-free medium.

Initially, we reduced the serum stepwise in adherent culture in T flasks from 10% to 2%, and then we switched to suspension culture in Erlenmeyer flasks, gradually reducing the serum from 2% to serum-free.

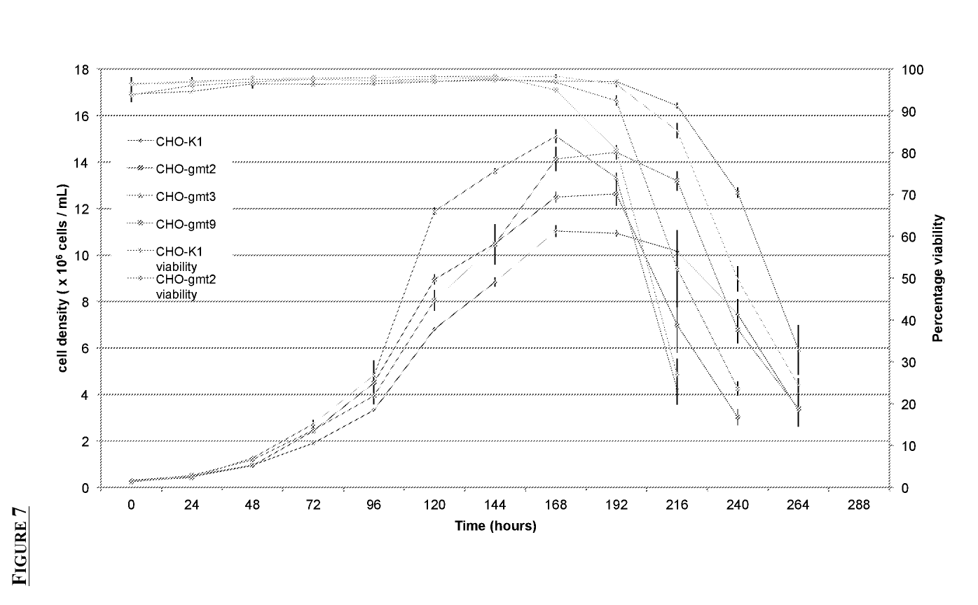

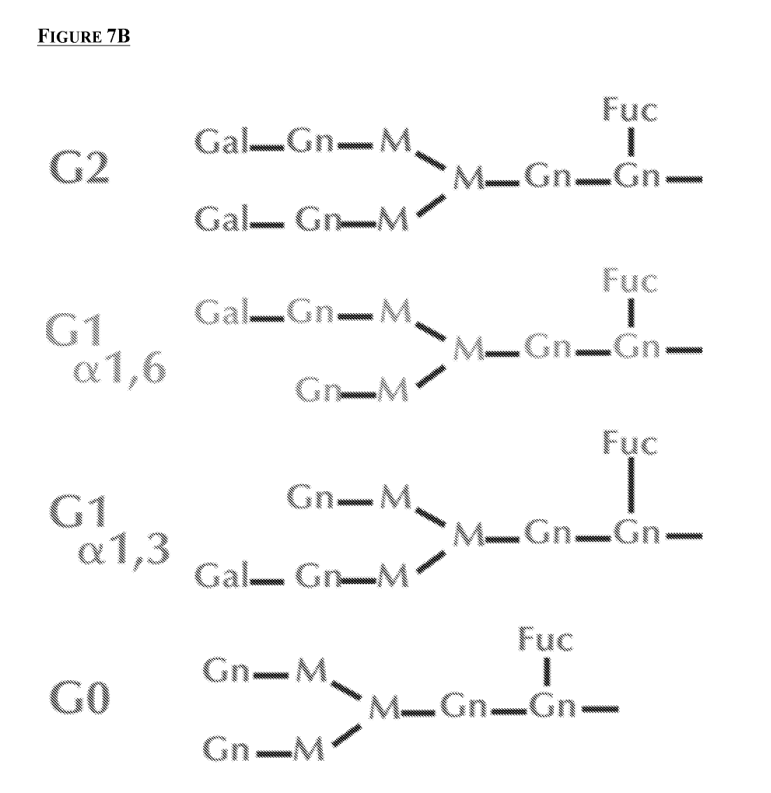

Once fully adapted, we carried out a comparison of growth curve between the four cell lines over 11 days in batch culture, with the results as shown in FIG. 7. The maximum cell density for CHO-K1, CHO-gmt2, CHO-gmt3, and CHO-gmt9 were 11×10⁶, 12.6×10⁶, 15×10⁶, and 13×10⁶ cells per ml medium, respectively.

As anticipated, CHO-gmt3, the cell line with the fastest and highest growth rate, had the fastest decline in viability, reaching below 50% on day 9. The viability of CHO-K1 and CHO-gmt9 dropped below 50% on day 11, while the viability of CHO-gmt2 dropped below 50% on day 10.

### Example 18

### Discussion

The N-glycans attached to the Fc region of recombinant human IgG1 antibodies produced by CHO cells are often heterogeneous. It represents a major challenge for the development and production of follow-on biologics (biosimilars).

Even for the production of the innovators, it is not an easy task to maintain batch-to-batch consistency.

The Fc N-glycans attached to the same antibody can differ significantly when produced by different stably transfected CHO lines. The differences can also be found in the antibodies produced by the same cell line but in different batches due to variations in culture conditions.

These differences can be attributed to the fact that glycosylation is not a template driven process and many genes and factors can affect glycosylation reactions. To minimize the batch-to-batch inconsistency in glycosylation profiles is a serious regulatory concern because of quality assurance (van Berkel et al., 2009; Schiestl et al., 2011).

The Fc N-glycans produced by CHO cells mainly consist of core-fucosylated biantennary complex type, containing zero or one galactose residue (G0F or G1F), with a small amount of N-glycans containing 2 galactose residues (G2F). The relative amount of G0F in total N-glycans can vary between 40˜70% and is the main reason for heterogeneity and inconsistence. A small amount of Fc N-glycans produced by CHO cells contains sialic acid.

We previously used a cytotoxic lectin, Maackia amurensis agglutinin (MAA), and isolated CHO mutant cell line CHO-gmt1 (also called MAR-11) that lacks functional CMP-sialic acid transporter, resulting in asialylated N-glycans (Lim et al., 2008). Antibodies produced in CHO-gmt1 cells contain no sialic acid on their N-glycans.

In the current study, we used the same lectin and isolated another CHO mutant. This mutant lacks functional UDP-galactose transporter gene (Slc35a2) and it has been named CHO-gmt2 cells.

This mutant cell line produces N-glycans that lack galactose, hence terminating with N-acetylglucosamine. The glycan profile of CHO-gmt2 is similar to that of CHO Lec8 cells, which also lacks functional Slc35a2 (Deutscher and Hirschberg, 1986; Oelmann et al., 2001).

As we have shown in this report, the N-glycans attached to the EPO-Fc produced by CHO-gmt2 cells are mainly core-fucosylated galactose-free complex type. The N-glycans attached to the antibody produced by CHO-gmt2 cells are mainly G0F.

Therefore, the heterogeneity of the Fc N-glycans has been significantly reduced and CHO-gmt2 mutants have the potential as host cells to produce antibodies with significantly reduced glycan heterogeneity and inconsistency.

Removal of core fucose from Fc N-glycans was shown to increase its binding affinity to FcγRIII and thereby enhance its activity to activate ADCC. A total of eleven fucosyltransferases have been characterized and only FUT8 is a α6trnasferase and able to transfer fucose to the core position on N-glycans (Becker and Lowe, 2003). Therefore, Fut8 has been knocked out by homologous recombination for producing fucose-free antibodies (Yamane-Ohnuki et al., 2004).

We inactivated the GDP-fucose transporter (Slc35c1) gene in CHO-K1 cells for producing fucose-free antibodies. CHO-gmt3 was isolated using ZFNs targeting the Slc35c1 gene in wild-type CHO-K1. Similarly, CHO-gmt9 was isolated from CHO-gmt2 cells using the same set of ZFNs.

The resulting CHO-gmt9 lacks both Slc35a1 and Slc35c1 gene products, hence more than 90% of the N-glycans attached to the IgG antibody are galactose-free and fucose-free G0F0. This eliminates the issue of heterogeneity and batch-to-batch inconsistency in the production of recombinant antibodies.

The CHO-K1 transcriptome data have shown that among all fucosyltransferases, only fucosyltransferases FUT8 and protein O-fucosyltransferases (POFUT-1 and POFUT-2) are expressed (Xu et al., 2011). As both POFUT-1 and POFUT-2 are localized in the ER, not in the Golgi (Loriol et al., 2006), the only fucosyltransferase that is affected by inactivating the Golgi GDP-fucose transporter gene (Slc35c1) is FUT8. The activities of POFUT-1 and POFUT-2 will not be affected as their substrate, GDP-fucose, is transported into the ER by the ER GDP-fucose transporter gene (Slc35c2) (Lu et al., 2010), which is not affected in CHO-gmt3 and CHO-gmt9. Therefore, inactivating the FUT8 gene or the GDP-fucose transporter gene (Slc35c1) should have exactly the same impact on CHO-K1 cells.

A recent study showed that agalactosylated HIV-specific antibodies were associated with antiviral activity and spontaneous control of HIV infection (Ackerman et al., 2013), while agalactosylated IgG1 anti-erithrocyte autoantibodies possessed enhanced pathogenic effect (Ito et al., 2014). Moreover, it is known that removal of fucose from the IgG glycan results in more improved ADCC (Shields et al., 2002; Shinkawa et al., 2003). It is also interesting to note that adding bisecting GlcNAc in the N-glycan of IgG by overexpression of GnTIII has also been shown to increase ADCC activity (Davies et al., 2001). Furthermore, it was shown that this was also due to better binding affinity to the receptor FcγRIII, as is the case with afucosylated IgGs.

Since it is in our interest to use these mutant cell lines to commercially produce recombinant proteins in bioreactor settings, we gradually adapted the cell lines into serum free suspension cultures and performed an experiment to determine their growth profile.

As can be seen in FIG. 7, the mutant cell lines had comparable growth rate to the wild type CHO-K1, and could reach similar maximum cell density of about 10⁷ cells per ml. This demonstrated that, at least in batch culture conditions, the genetic defects in the glycosylation pathway did not affect growth profile.

One issue that is commonly encountered during adaptation of cell lines to serum free suspension cultures is the change in glycosylation profile (Costa et al., 2013). This is of utmost importance especially if the cell lines are intended for production of antibodies. It was found that in intermediate levels of serum (2.5 to 0.15%), galactosylation and sialylation levels increased, while fucosylation level decreased.

In contrast, in the final stages of adaptation (0.075 to 0% serum), the inverse was observed (Costa et al., 2013). Under normal circumstances, these observations necessitate product quality control and assurance at the early stages of development. However, as the mutant cell lines already possess genetic defects in galactosylation (and hence terminal sialylation) and fucosylation, this issue can be avoided altogether.

### Example 19

### Inactivation of GDP-Fucose Transporter Gene (Slc35c1) in CHO Cells—Introduction

Recombinant human IgG1 antibodies used to target cancer cells are commonly produced in Chinese hamster ovary (CHO) cells. Both the Fab and Fc regions of the antibodies are required to carry out these activities. Binding of target antigen on cancer cells by the Fab region is followed by the engagement of the Fc region with the Fc receptor, FcγRIII, expressed on natural killer (NK) cells to kill the cancer cells via the antibody-dependent cellular cytotoxicity (ADCC) mechanism. Major sites of the Fc interaction with the FcγRIII are located in the hinge region and the CH2 domains of the antibody [1, 2]. In particular, this Fc-FcγRIII interaction is significantly affected by the glycan structures present at the conserved N-glycosylation site Asn²⁹⁷ in each of the CH2 domains [3]. It is now widely recognized that removal of the core fucose from the N-glycans of human IgG1 antibody significantly enhances its affinity towards FcγRIIIa, and therefore dramatically improves its ADCC activity [4, 5]. Enhanced in vivo ADCC by fucose-free antibodies has also been demonstrated in animal models and patients [6-9].

In mammalian cells, fucosylation reactions that take place in the Golgi apparatus frequently modify the N- and O-linked glycans, resulting in the formation of core fucose and the Lewis blood group antigens. Biochemical inhibitors for fucosylation reactions such as 2-fluorofucose and 5-alkynylfucose derivatives have been used to generate fucose-free antibodies [10]. Lec13 mutant CHO cells exhibit reduced activity of the GDP-mannose-4,6-dehydratase (GMD) and therefore reduced levels of GDP-fucose and fucosylated glycans [11]. In mammalian cells, GDP-fucose can be synthesized by two distinct pathways [12]. Loss-of-function mutations in the GMD gene can only affect the de novo pathway, not the salvage pathway. Therefore, Lec13 cells can produce IgG with reduced fucose content, not fucose-free antibodies.

Although a total of 13 fucosyltransferases have been identified in mammalian genome, only α1,6 fucosyltransferase (FUT8) is able to transfer the core fucose to the N-glycans [12]. Consequently, FUT8 gene in CHO cells has been inactivated by homologous recombination [13] and zinc-finger nucleases (ZFNs) [14]. The substrate for fucosylation reactions, GDP-fucose, is synthesized in the cytosol and has to be transported into the Golgi or the endoplasmic reticulum (ER) by specific transporters in order to serve as the substrate for fucosylation reactions. The GDP-fucose transporter encoded by the Slc35c1 gene transports GDP-fucose into the Golgi for core fucosylation. Meanwhile, a putative ER GDP-fucose transporter is believed to be encoded by Slc35c2 gene [15]. Thus inactivation of Slc35c1 gene can be an alternative strategy to prevent core fucosylation of N-glycans. It has been shown that loss-of-function mutation in the Golgi GDP-fucose transporter gene was able to eliminate all fucosylation reactions that occur in the Golgi [16, 17]. Therefore, we decided to inactivate the GDP-fucose transporter gene in CHO cells for production of fucose-free antibodies.

Three genome editing technologies developed recently have simplified the process of engineering cells for biologics production. Zinc-finger nucleases (ZFNs) are artificial restriction enzymes generated by fusion of zinc-finger DNA-binding domains normally found in transcription factors to the cleavage domain of restriction enzyme FokI [18-20]. The DNA-binding domain of ZFNs generally consists of three or four zinc-finger units. Each zinc-finger unit recognizes a 3-base pair (bp) stretch of DNA in the chromosome. Specificity of the ZFNs is determined by a motif of 7 amino acids within each zinc-finger. In order to allow the two FokI cleavage domains to dimerize and generate double-strand breaks (DSBs) in the chromosomal DNA, the two ZFNs must bind the opposite strands of the DNA and the two binding sites have to be separated by 5-7 bps. The DSBs created in the genomic DNA can then be repaired by error-prone non-homologous end joining (NHEJ) pathway which can create deletion or insertion mutations.

TALENs consist of customized transcription activator-like effector (TALE) fused to the catalytic domain of FokI nuclease [21, 22]. TALEs belong to a large family of transcription factors derived from plant pathogenic bacteria Xanthomonas spp. The DNA-binding domain of TALEs consists of several tandem repeats of 34 amino acids. Sequences of TALE repeats are highly conserved and differ mainly in two amino acid residues at positions 12 and 13, known as the repeat variable di-residues (RVDs). TALEs observe a simple cipher for DNA recognition where each repeat independently binds one nucleotide in the target DNA sequence as specified by the RVDs (where NI=A, HD=C, NG=T, NN=G or A) [23, 24]. Like ZFNs, TALENs function as a pair by binding to target DNA sequence on opposite DNA strands separated by a spacer of 12-21 bps and generate a DSB at the target site [22, 25].

The CRISPR (clustered regularly interspaced short palindromic repeats)-Cas9 (CRISPR-associated nuclease 9) system is a new technology for genome engineering. Unlike ZFNs and TALENs, Cas9 nuclease of the CRISPR-Cas9 system is recruited to the target site by a short guide RNA (gRNA). To inactivate a specific gene in mammalian cells, the cells are transfected by a vector that contains two transcription units. The expression of the short gRNA is controlled by a Pol III promoter such as U6 promoter and the synthesis of the Cas9 protein is controlled by a Pol II promoter such as CMV promoter. The gRNA associates itself with Cas9 protein and hybridizes with the target DNA in a sequence-specific manner by Watson-Crick base pairing. Each of the two active sites of Cas9 cleaves one strand of DNA to generate a DSB. In principle, any genomic region that matches the GN₂₀GG sequence (where “N” can be any nucleotide) can be targeted by the CRISPR-Cas9 system [26-28].

In this paper, we report the inactivation of Slc35c1 gene in CHO cells using ZFNs, TALENs and CRISPR-Cas9. Mutant cells generated by the different technologies were enriched and isolated by fluorescence-activated cell sorting (FACS) coupled with a fucose-specific Aleuria aurantia lectin (AAL). This approach dramatically improved the efficiency of isolating mutant cells. A model glycoprotein, erythropoietin-Fc (EPO-Fc) fusion protein, produced by the mutant cells completely lacked the core fucose residues on their N-glycans. As ZFNs, TALENs and CRISPR-Cas9 technologies are all known to be able to generate off-target mutations which may affect cell growth and antibody productivity, the three technologies were also used to inactivate the same Slc35c1 gene in a pre-existing anti-Her2 antibody-producing CHO cell line. The growth rates and anti-Her2 antibody productivities of mutant pools generated by the three technologies were compared.

### Example 20

### Inactivation of GDP-Fucose Transporter Gene (Slc35c1) in CHO Cells—Materials and Methods

Biotinylated AAL was purchased from Vector Laboratories (Burlingame, Calif.). Cy3-conjugated streptavidin was purchased from Jackson ImmunoResearch Laboratories Inc. (West Grove, Pa.). Trypsin was purchased from Promega Biosciences (San Luis Obispo, Calif.). PNGase F was purchased from Prozyme Inc. (San Leandro, Calif.). Hypercarb SPE cartridges and N-linked oligosaccharide standards were from Dextra Laboratories (Reading, UK). 2,5-Dihydroxybenzoic acid and Sep-Pak Vac C18 cartridge were from Waters Corporation (Milford, Mass.). Sodium acetate, ammonium carbonate, acetonitrile, methanol and sodium hydroxide were all of analytical grade from Merck KGaA (Darmstadt, Germany). Ultrapure water system from Sartorius (Goettingen, Germany) was utilized for analysis.

### Example 21

### Inactivation of GDP-Fucose Transporter Gene (Slc35c1) in CHO Cells—Materials and Methods—Cells and Cell Culture

CHO-K1 cells were obtained from the American Type Culture Collection (ATCC). CHO-K1 cells and CHO-gmt3 mutant cells were grown in Dulbecco's Modified Eagle's Medium (DMEM) from Life Technologies (Carlsbad, Calif.) supplemented with 10% FBS (Life Technologies), at 37° C. with 5% CO₂. A trastuzumab (Herceptin)-producing CHO DG-44 cell line (CHO-HER) was generated as described previously [29]. CHO-HER and CHO-HER mutant pools with inactivated GDP-fucose transporter gene generated by ZFNs, TALENs and CRISPR-1 were cultured as 25 ml suspension culture in 125 ml shake flasks with chemically defined serum-free growth medium. The growth medium comprises HyClone™ PF-CHO™ (Thermo Scientific, Waltham, Mass.) and CD CHO (Life Technologies) at a 1:1 ratio, supplemented with 2 g/l sodium bicarbonate. The medium was also supplemented with 6 mM glutamine (Life Technologies), and 0.05% Pluronic F-68 (Life Technologies). Cell density and viability were measured using the Trypan Blue exclusion method on an automated Vi-CELL XR Cell viability Analyzer from Beckman Coulter, Inc. (Brea, Calif.). Adherent cells were adapted to suspension cells via the in-house adaptation protocol through gradual reduction of serum into a serum-free protein-free medium.

### Example 22

### Inactivation of GDP-Fucose Transporter Gene (Slc35c1) in CHO Cells—Materials and Methods—Generation of ZFN Constructs to Target the Slc35c1 Gene in CHO Cells

The “modular assembly” method was used to generate the specific left and right zinc-finger nucleases (ZFNs) for targeting the Golgi GDP-fucose transporter (Slc35c1) gene in CHO cells. As described earlier [30], a DNA sequence in the first exon of the GDP-fucose transporter coding region, 5′-tAACCTCTGCCTCAAGTACGTAGGGGTGGCCt-3′, was identified as the target site for ZFNs. The sequence of the seven amino acids in each zinc finger motif that determine the specificity of each finger was designed based on publically available information. The two ZFNs, ZFN-L and ZFN-R, used in this study were the same as those used in the previous report [30].

### Example 23

### Inactivation of GDP-Fucose Transporter Gene (Slc35c1) in CHO Cells—Materials and Methods—Generation of TALEN Constructs to Target the Slc35c1 Gene in CHO Cells

Transcription activator-like effector (TALE) repeat monomers (NI, NG, HD, NH) were synthesized as gBlocks (Integrated DNA Technologies (IDT) Inc., Coralville, Iowa) and cloned into pCR-Blunt II-TOPO® (Life Technologies) as templates for subsequent PCR reactions. The DNA sequences of these repeat monomers are derived from the 34 amino acid TALE repeat of Xanthomonas sp. AvrBs3 gene [31] and are codon-optimized for expression in CHO cells and also to reduce repetitiveness in assembled TALENs. Optimization of the sequence was carried out using the online IDT Codon Optimization tool (http://sg.idtdna.com/CodonOpt). Codon-optimized DNA fragments consisting of a nuclear localization signal in front of the truncated TALE N-terminal 136 amino acids (Δ152) of AvrBs3, and a 0.5 TALE repeat linked to +63 amino acid truncated C-terminal domain based on a previous report [22] were individually synthesized as gBlocks. These fragments were cloned sequentially into the multiple cloning site (MCS) of a modified pVax1 vector together with one of the two enhanced FokI domains to form the destination vector. Our destination vector uses a 25-bp sequence in place of the ccdB gene. FokI domains used are obligate heterodimers containing both Sharkey [32] and ELD:KKR [33] mutations for enhanced cleavage activity.

TALENs with 18.5 repeats were generated based on a modified Golden-Gate cloning methodology [34]. TALE repeats were PCR-amplified with position-specific primers to generate a library of monomers flanked by BsmBI and BsaI sites. The monomers were first assembled as hexamers into the array vector (a modified pcDNA3.1(+) vector containing BsmBI sites at the MCS and lacking BsaI sites) in a Golden Gate cloning reaction using BsmBI enzyme (Thermo Scientific) and T7 DNA ligase (New England Biolabs, Ipswich, Mass.). Hexamers were then assembled as an 18-mer into the destination vector between the TALE N-terminus and 0.5 repeat in a Golden-Gate cloning reaction using BsaI enzyme and T7 DNA ligase (New England Biolabs). This generates a fully assembled TALEN consisting of the TALE N-terminus, an 18.5-mer TALE repeat DNA binding domain and the TALE C-terminus linked to FokI domain.

To design a TALEN pair to target CHO cell Slc35c1 gene, we analyzed the DNA sequence using the online tool TAL Effector Nucleotide Targeter 2.0 (https://talent.cac.cornell.edu/node/add/talen) [35, 36]. A potential target site in the first exon of the GDP-fucose transporter coding region was identified. It consists of two 20-bp TALE binding sites separated by a 19-bp spacer. The 34 amino acid TAL repeats used is of the form LTPEQVVAIASXXGGKQALETVQRLLPVLCQAHG where the underlined amino acids in the 12^{th} and 13^{th} position refer to the repeat variable di-residue (RVDs). The RVDs used for the TALENs are as follows: Left TALEN: NI HD HD NG NH HD NG NH NH NI HD NI NH HD HD HD HD NG HD; Right TALEN: NH NH NG NI NH NI NI NI NH NG NH NI HD NH NI NI NH NI NG.

### Example 24

### Inactivation of GDP-Fucose Transporter Gene (Slc35c1) in CHO Cells—Materials and Methods—Generation of CRISPR-Cas9 Constructs to Target the Slc35c1 Gene in CHO Cells

GeneArt® CRISPR Nuclease Vector kit was purchased from Life Technologies. Two CRISPR-Cas9 target sequences, both located in the first exon of the coding region in the CHO cell Slc35c1 gene, were selected based on previous publications [26, 28]. To construct CRISPR-1 vector, the forward oligonucleotide was 5′-CGGGCGCTGCAGATCGCGCGTTTT-3′, and the reverse oligonucleotide was 5′-GCGCGATCTGCAGCGCCCGCGGTG-3′. To construct CRISPR-2 vector, the forward oligonucleotide was 5′-TGCAAGGGCCTCAGCACTCGTTTT-3′, and the reverse oligonucleotide was 5′-GAGTGCTGAGGCCCTTGCACGGTG-3′. Each pair of oligonucleotides was annealed and cloned into GeneArt® CRISPR Nuclease Vector by following manufacturer's instructions.

### Example 25

### Inactivation of GDP-Fucose Transporter Gene (Slc35c1) in CHO Cells—Materials and Methods—Transfection of CHO-K1 Cells and Anti-Her2 (Trastuzumab) Antibody-Producing CHO DG44 Cells (CHO-HER) with ZFNs, TALENs or CRISPR-Cas9 Constructs

CHO-K1 cells were first seeded overnight at 6×10⁵ cells per well of 6-well plates. Constructs expressing ZFNs or TALENs were transiently transfected into CHO-K1 cells using Lipofectamine LTX (Life Technologies) according to manufacturer's protocol. For each transfection, 2.5 μg of plasmid DNA (1.25 μg for each ZFN or TALEN) with 2.5 μl Plus reagent were mixed with 9 μl of Lipofectamine LTX reagent in 200 μl of Opti-MEM® I Reduced Serum Medium (OPTIMEM) (Life Technologies) and added to the cells in each well containing 2 ml of medium. Transfection medium was replaced with fresh culture medium at 8 hours post-transfection and cells were cultured for 3 days. Cells were then scaled up to T-75 flask and grown for another 3 days before undergoing AAL-staining and FACS.

Anti-Her2 (trastuzumab) antibody-producing CHO DG44 cells (CHO-HER) were transfected with ZFNs or TALENs targeting GDP-fucose transporter using 4D-Nucleofector™ (Lonza, Cologne, Germany) system according to manufacturer's protocol. Briefly, 1.5 million cells were harvested from exponentially growing suspension culture and washed with Dulbecco's phosphate buffered saline (DPBS) (Life Technologies). The cells were suspended in 100 μl of SG cell line Nucleofector™ solution containing 5 μg of plasmid DNA (2.5 μg for each ZFN or TALEN) in a Nucleocuvette™. Electroporation was then carried out using a Lonza 4D-Nucleofector™ X unit. Following transfection, cells were incubated in suspension in a 3 ml CD CHO:HyClone PF CHO medium in a 6-well plate overnight before they were transferred into a 25 ml fresh medium in a 125-ml shake flask for further culture. Cells were then harvested for AAL-staining and FACS at 7-10 days post-transfection.

For CRISPR-Cas9 modification, 5 million suspension CHO-K1 cells were electroporated using 4D-Nucleofector™ system with 5 μg of CRISPR-Cas9 plasmids in 100 μl of SG cell line Nucleofector™ solution. Following transfection, cells were recovered in growth medium in a suspension 6-well plate overnight before being scaled up to shake flask culture. Cells were then harvested for AAL-staining and FACS at 7-10 days post-transfection.

### Example 26

### Inactivation of GDP-Fucose Transporter Gene (Slc35c1) in CHO Cells—Materials and Methods—T7 Endonuclease 1 (T7E1) Mismatch Assay

T7 endonuclease 1 (T7E1) was used to detect mutations mediated by ZFNs, TALENs and CRISPRs as described previously [37]. Genomic DNA of CHO cells transfected with ZFNs, TALENs or CRISPRs was extracted using DNeasy Blood & Tissue Kit (Qiagen, Hilden, Germany) at 72 hours post-transfection. PCR amplification of Slc35c1 gene region encompassing the target sites was carried out for 35 cycles (95° C., 30 s; 60° C., 30 s; 68° C., 40 s) using AccuPrime Taq DNA Polymerase High Fidelity Kit (Life Technologies) and the primer pair 5′-CCGTGGGGTGACCTAGCTCTT-3′ and 5′-GCCACATGTGAGCAGGGCATAGAAG-3′. Purified PCR products were then heated and re-annealed slowly for heteroduplex formation. The reannealed DNA were then treated with 5 U of T7E1 (New England Biolabs) for 15 mins at 37° C. and resolved on 2.5% TBE agarose gel. The gels were stained with RedSafe™ staining solution (iNtRON Biotechnology, Gyeonggi, Korea) and analyzed on Gel Doc™ EZ imager using Image Lab™ Software (Bio-Rad). Gene modification activities were calculated using the formula % gene modification=100×(1−(1−fraction cleaved)^{1/2}) as described previously [38].

### Example 27

### Inactivation of GDP-Fucose Transporter Gene (Slc35c1) in CHO Cells—Materials and Methods—Mutant Cells in Transfected CHO Cells were Enriched and Isolated by FACS

To prepare for FACS, 1×10⁷ transfected cells were harvested from a T-75 flask or 125-ml shake flask and washed twice with cold DPBS (Life Technologies). Blocking was carried out in 1% BSA/PBS (Sigma-Aldrich) for 30 min to prevent non-specific binding. Cells were then stained with 2 μg/ml biotinylated AAL (Vector Laboratories) in 1% BSA/PBS for 35 min. After two washes with cold DPBS, the stained cells were incubated with Cy3-conjugated streptavidin (Jackson ImmunoResearch Laboratories Inc.) at 2 μg/ml in 1% BSA/PBS for 35 min. After two more washes with cold DPBS, the cells were re-suspended in 2 ml of FACS buffer (DPBS containing 2% FBS) at a concentration of 5×10⁶ cells/ml prior to sorting. All incubation steps listed above were carried out on ice and sterile reagents were used. Cells were then subjected to FACSAria III cell sorter (Becton Dickinson Biosciences, Mountain View, Calif.). Single and viable cells were gated for by excluding debris and doublets on a series of forward scatter and side scatter dot plots (FSC-A vs. SSC-A, SSC-H vs. SSC-W, FSC-H vs. FSC-W). Cy3 signal was emitted using a blue laser (488 nm) excitation and fluorescence detected after passing through a bandpass filter 575/26 and 550 LP (Longpass) mirror. Sorted cells were collected and cultured in fresh medium supplemented with Antibiotic-Antimycotic (Life Technologies). Antibiotic-Antimycotic supplementation was then removed after 1 week of culture. For FACS of suspension CHO-K1 and CHO-HER cells, about 2-5×10⁴ cells were collected into a single well of a 24-well plate containing 1 ml of growth medium containing serum and Antibiotics-Antimycotics. The sorted cells were allowed to recover in serum-containing medium for 4 days before they were scaled up into serum-free medium in 125-ml shake flask.

### Example 28

### Inactivation of GDP-Fucose Transporter Gene (Slc35c1) in CHO Cells—Materials and Methods—Molecular Characterization of Mutant Clones

Genomic DNA from mutant clones was extracted using DNeasy Blood & Tissue Kit (Qiagen). PCR amplification of the targeted sites in the CHO cell Slc35c1 gene was carried out for 28 cycles (95° C., 30 s; 60° C., 30 s; 68° C., 45 s) using AccuPrime Pfx DNA Polymerase (Life Technologies) and the primer pair 5′-CCGTGGGGTGACCTAGCTCTT-3′ and 5′-GCCACATGTGAGCAGGGCATAGAAG-3′. PCR products were gel-purified and cloned into pCR-Blunt II-TOPO® Vector (Life Technologies). 10 bacterial clones from each TOPO reaction were sequenced to characterize the mutation.

### Example 29

### Inactivation of GDP-Fucose Transporter Gene (Slc35c1) in CHO Cells—Materials and Methods—Genetic Complementation of GDP-Fucose Transporter-Deficient Mutant Clones

To check the phenotype of mutant clones, 4×10⁵ cells were seeded in 6-well plates overnight and transfected using Lipofectamine LTX (Life Technologies) with 2 μg of plasmid DNA encoding human GDP-fucose transporter (Accession number: NM_018389). For suspension-grown CHO-K1 and CHO-HER cells, 5 million cells were electroporated with 5 μg of plasmid DNA encoding human GDP-fucose transporter in SG cell line Nucleofector™ solution using 4D-Nucleofector™ system. 1-2×10⁶ cells were harvested two days after transfection. Cells were stained with biotinylated AAL and labeled with Cy3-streptavidin as described earlier and analyzed on the FACSAria cell sorter.

### Example 30

### Inactivation of GDP-Fucose Transporter Gene (Slc35c1) in CHO Cells—Materials and Methods—Transient Expression of EPO-Fc in CHO-K1 and CHO-gmt3 for N-Glycan Analyses

Fc fusion protein of human erythropoietin (EPO-Fc) was produced in CHO-K1 and CHO-gmt3 cells as described earlier [39] for N-glycan structure analyses. Fc region from human IgG1 was fused to the C-terminus of EPO by overlap PCR. The PCR product for the EPO-Fc fusion was cloned into pcDNA3.1 with a Kozak sequence placed upstream of the translation start codon ATG. Cells were seeded overnight at 3×10⁵ cells per ml into ten T-175 flasks. EPO-Fc construct was transiently transfected into CHO-K1 and CHO-gmt3 cells using Lipofectamine 2000 (Life technologies) with 60 μg of plasmid DNA per flask. At 6 hours post-transfection, cells were washed twice with PBS and replaced with chemically defined serum-free growth medium. Conditioned media containing secreted recombinant EPO-Fc were collected every 2 days over the course of 6 days, purified with a HiTrap Protein A HP column using an FPLC AKTA Purifier (GE Healthcare, Pittsburgh, Pa.). An amount of 200 μg of purified EPO-Fc was used for carbohydrate structure analysis. The carbohydrates liberated from the EPO-Fc by PNGase F were analyzed by MALDI-TOF mass spectrometry analysis as described previously [40].

### Example 31

### Inactivation of GDP-Fucose Transporter Gene (Slc35c1) in CHO Cells—Materials and Methods—Glycan Release and Purification

N-glycans were released directly from intact, purified glycoprotein samples, by PNGase F treatment. Briefly, glycoprotein samples, recombinant EPO-Fc, the in-house produced anti-Her2 antibody (trastuzumab) or Herceptin (produced by Roche), were first desalted using a PD 10 column (GE Healthcare, Pittsburgh, Pa.) following manufacturer's protocol. Then, an aliquot of the glycoprotein (20 μg for EPO-Fc or 100 μg for the anti-Her2 antibody) was mixed with 500 U of the PNGase F in the reaction buffer in a total volume of 100 μl and incubated at 37° C. for 1 h. Such conditions will result in complete deglycosylation of IgG and EPO-Fc as indicated by SDS-PAGE-capillary gel electrophoresis (data not shown). The released glycans were then purified by HyperCarb porous graphitized carbon cartridge (Thermo Fisher Scientific, CA), and dried by CentriVap (Labconco, Kansas City, Mo.) for subsequent analyses by matrix-assisted laser desorption/ionization time-of-flight mass spectrometry (MALDI-TOF MS) and hydrophilic interaction liquid chromatography-ultra performance liquid chromatography combined with quadrupole time-of-flight (HILIC-UPLC-QTOF).

### Example 32

### Inactivation of GDP-Fucose Transporter Gene (Slc35c1) in CHO Cells—Materials and Methods—Glycan Profiling by MALDI-TOF MS

MALDI-TOF MS analysis of permethylated N-glycans was described in our previous report [29]. Briefly, the previously dried glycans were permethylated according to published protocols [41] and then cleaned up and fractioned into 15%, 35%, 50% and 75% (v/v) acetonitrile in water using a Sep-Pack C18 cartridge (Waters Corporation) [42]. Each elution fraction was dried under vacuum. Before MALDI-TOF MS acquisition, the dried permethylated glycan samples were dissolved in 30 μl of 80% (v/v) methanol in water. 0.5 μl of reconstituted sample was mixed with 0.5 μl of 2,5-dihydroxybenzoic acid (DHB) and then spotted onto the MALDI target plate. Mass spectra were acquired on a 5800 MALDI-TOF/TOF mass spectrometer (AB Sciex, Foster City, Calif.) in positive reflectron mode. Glycan structures were assigned to the respective peaks based on the matching mass-to-charge ratio (m/z) and knowledge of the N-glycan biosynthetic pathway in CHO cells.

### Example 33

### Inactivation of GDP-Fucose Transporter Gene (Slc35c1) in CHO Cells—Materials and Methods—Glycan Analysis by HILIC-UPLC-QTOF

Quantitative analysis of N-glycan was performed by HILIC-UPLC-QTOF on a Waters UNIFI Biopharmaceutical platform (version 1.7, Waters Corporation). Briefly, the previously dried N-glycans were labelled with 2-aminobenzamide (2-AB) according to a published protocol [41]. The excess 2-AB was removed by passing the labeling mixture through a MiniTrap G-10 desalting column (GE Healthcare) and the purified 2-AB-labeled glycans were then dried under vacuum. Before analysis, the dried samples were reconstituted in 250 μl of solvent consisting of 70% (v/v) acetonitrile in water. 10 μl of the reconstituted 2-AB glycan sample was then injected to the UNIFI Biopharmaceutical platform. The entire platform consists of a UPLC-H class ultra-performance liquid chromatogram (UPLC) which is online-connected to a Xevo G2-S quadrupole-time of flight (QTOF) mass spectrometer, both under the control of UNIFI Biopharmaceutical software platform (version 1.7). The UPLC-H class consists of a sample manager (kept at 10° C.), a quaternary pump, a column oven (kept at 40° C.) which houses a Waters BEH Glycan column (2.1 mm ID×150 mm length), and a fluorescence detector. Glycans were separated on the HILIC column using a binary solvent system. Solvent A is 50 mM ammonium formate (pH 4.4) and solvent B is acetonitrile. The analytical run takes place in 16 min by ramping up the solvent A from 30% to 47%. The column was then flushed with 80% solvent A before re-equilibrated with 30% A for the next run. Glycan signal was detected at excitation wavelength of 330 nm and emission wavelength of 420 nm. Raw retention time of each chromatographic peak was converted to a glucose unit (GU) by fitting into a calibration curve established by a 2-AB-labeled dextran ladder (Waters Corporation). The GU value of each chromatographic peak was then used to search against an experimental database for N-glycans embedded in the UNIFI Biopharmaceutical platform. Primary assignment was done by alignment of observed and the expected GU values. In case of structural ambiguity, i.e. a GU value corresponding to more than one structure within the error tolerance (typically 0.1 GU), decision was then made based on accurate mass confirmation (5 ppm error) by the online ESI-QTOF and the possible biosynthetic pathway of N-glycans in CHO cells. The analytes were introduced into the QTOF under the following conditions: cone voltage: 80 kV; capillary voltage: 2.75 kV; source temperature: 120° C.; desolvation gas flow: 800 L/h; desolvation temperature: 300° C. The QTOF was operated by scanning the mass range of 400-3,000 amu at the acquisition speed of 1 Hz. Mass accuracy was maintained by introducing a “lock spray” of Glu-fibrinopeptide (m/z=785.8421).

### Example 34

### Inactivation of GDP-Fucose Transporter Gene (Slc35c1) in CHO Cells—Materials and Methods—Exoglycosidase Digestions

The 2-AB-labeled glycans were digested with an array of exoglycosidases in 50 μl of 50 mM sodium acetate buffer (pH 5.5) for 18 h at 37° C. according to a published protocol [43]. After the incubation, enzymes were removed by passing the mixture through a centrifugal filter cartridge with 10 (kDa) nominal molecular weight limit (NMWL) cut-off (Merck Millipore, Cork, Ireland). Digested, 2-AB-labeled glycans were collected in the flow-through and analyzed by the HILIC-UPLC-QTOF system.

### Example 35

### Inactivation of GDP-Fucose Transporter Gene (Slc35c1) in CHO Cells—Materials and Methods—Growth and Productivity Analyses

CHO-gmt3 cells were first adapted to protein-free suspension shake flask culture. Viable cell density and cell viability of these mutant cell lines were examined. Cells were seeded at 2.5×10⁵ cells/ml in a 30 ml batch culture in triplicate samples and 1 ml fractions were collected at approximately 24-hour intervals for cell count. Cell density and viability from each sample were measured in duplicate using the Trypan blue exclusion method on an automated Vi-CELL XR Cell viability Analyzer (Beckman Coulter, Inc.). Growth and antibody titer (for CHO-HER cells) were analyzed until viability dropped below 50%. Antibody concentrations in 200 μl culture fractions were determined using the nephelometric method on an IMMAGE 800 immunochemistry system (Beckman Coulter, Inc.). Standard deviation (sd) was obtained from the cell count number for triplicate samples. Growth curve was plotted using mean±sd.

### Example 36

### Inactivation of GDP-Fucose Transporter Gene (Slc35c1) in CHO Cells—Results—Construction of ZFNs to Target the Slc35c1 Gene in CHO Cells

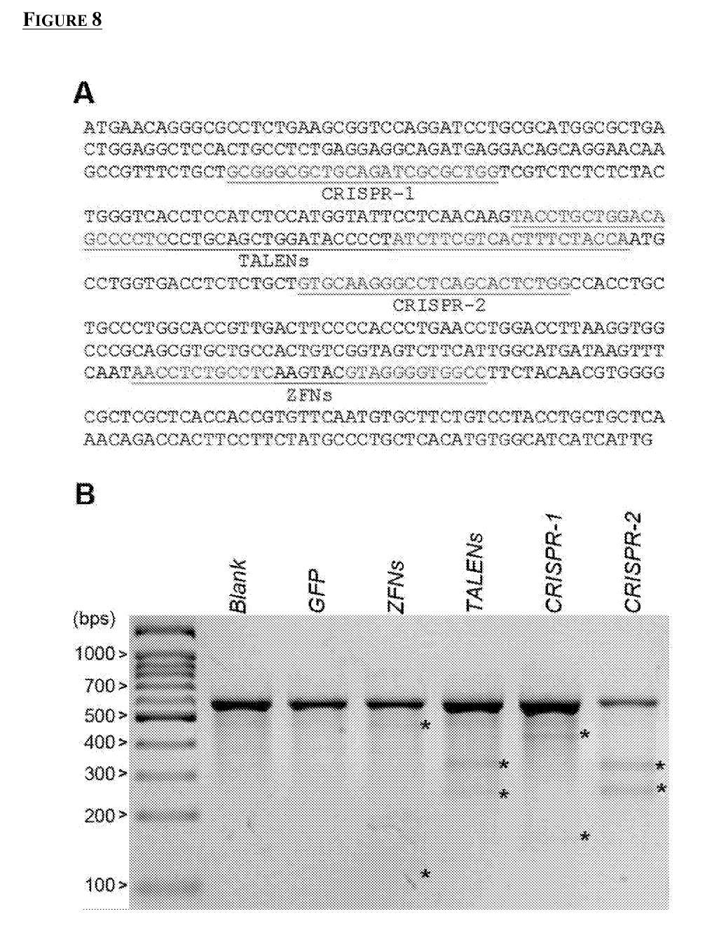

Based on published literature [18-20, 44-47], we designed a pair of ZFNs to target the Slc35c1 gene in a CHO cell line [30]. A suitable target sequence was identified in the first exon of the coding region of the Slc35c1 gene in CHO cells (FIG. 8A). In the current work, the two ZFNs for targeting the Slc35c1 gene were the same as those used in the previous report [30].

### Example 37

### Inactivation of GDP-Fucose Transporter Gene (Slc35c1) in CHO Cells—Results—Construction of TALENs to Target the Slc35c1 Gene in CHO Cells

To design a TALEN pair to target the Slc35c1 gene in CHO cells, we analyzed the DNA sequence using the online tool TAL Effector Nucleotide Targeter 2.0 (https://talent.cac.cornell.edu/node/add/talen) [35, 36]. A suitable TALEN target site was identified in the first exon of the coding region (FIG. 8A). The 34-amino acid TAL repeats used are LTPEQVVAIASXXGGKQALETVQRLLPVLCQAHG where the RVDs (underlined) were NI, NG, HD and NH for binding A, T, C and G, respectively [23, 24, 48]. The DNA sequences of these repeat monomers were codon-optimized for expression in mammalian cells. Based on Miller's architecture [22], TALENs with 18.5 RVDs (having 20-bp specificity) were assembled by the Golden-Gate methodology with minor modifications as described in Materials and Methods [34]. The FokI domains contained both Sharkey [32] and ELD:KKR [33] mutations for enhanced cleavage activity.

### Example 38

### Inactivation of GDP-Fucose Transporter Gene (Slc35c1) in CHO Cells—Results—Construction of CRISPR-Cas9 Expression Constructs to Target the Slc35c1 Gene in CHO Cells

Several sites in the first exon of the coding region in the Slc35c1 gene matched the GN₂₀GG sequence and therefore can be selected as target sites for CRISPR-Cas9 [26, 28]. In this work, two CRISPR-Cas9 target sites were selected (FIG. 8A). For CRISPR-1, the target site is GCGGGCGCTGCAGATCGCGCTGG. For CRISPR-2, the target site is GTGCAAGGGCCTCAGCACTCTGG. One pair of oligonucleotides was synthesized for each CRISPR-Cas9 site. The oligonucleotides were annealed and cloned into GeneArt® CRISPR Nuclease Vector.

### Example 39

### Inactivation of GDP-Fucose Transporter Gene (Slc35c1) in CHO Cells—Results—Mismatch Assay to Evaluate Targeted DNA Cleavage Efficiency by the ZFNs, TALENs and CRISPRs

T7 endonuclease 1 (T7E1) was used to evaluate the cleavage activities mediated by the ZFNs, TALENs and CRISPRs as described previously [37]. Genomic DNA of CHO cells transfected with ZFNs, TALENs or CRISPRs was extracted 72 hours after transfection. The Slc35c1 genomic region encompassing the target sites was amplified by PCR. Purified PCR products were then heated and reannealed slowly to permit the formation of heteroduplex between the mutated DNA strand and the wild-type DNA strand. The reannealed DNA samples were then treated with T7E1 and resolved on agarose gel (FIG. 8B). The expected sizes of the cleaved fragments are as indicated by the asterisk (*). The gene modification activities of TALEN, CRISPR-1 and CRISPR-2 were calculated to be 7.3%, 6.4% and 18.4%, according to the method by Guschin et al. [38]. The results suggest that CRISPR-2 has the highest gene modification activity. TALENs and CRISPR-1 are similarly efficient in generating mutations as the intensity levels of the cleaved DNA fragments are similar. ZFNs designed in this study appear to be the least efficient in generating mutations as suggested by the mismatch assay, as a result it was difficult to calculate the cleavage percentage.

### Example 40

### Inactivation of GDP-Fucose Transporter Gene (Slc35c1) in CHO Cells—Results—The FACS Approach to Enrich and Isolate GDP-Fucose Transporter Deficient Mutants Generated by Three Different Genome Editing Approaches

In our previous report [30], single clones were isolated two days after transfection with the plasmids encoding the ZFNs and screened by genomic PCR. Sequence analysis of the target locus was performed to identify mutations. This approach is both time consuming and labor intensive, and in many clones only one allele of the target gene is mutated. Therefore, we developed a lectin-based FACS method to enrich and isolate the mutants. The lectin, Aleuria aurantia lectin (AAL) that recognizes the core fucose on N-glycans [49] was used to identify mutant cells as the cells with fucose-free phenotype will not be stained by AAL.

To inactivate GDP-fucose transporter, wild-type CHO-K1 cells growing in a 6-well plate were first transfected with the plasmids encoding ZFNs, TALENs or CRISPRs. Two days after transfection, the cells were transferred into a T-75 flask and cultured to confluence.

To prepare for FACS, 1×10⁷ transfected cells were incubated with biotinylated AAL and followed by Cy3-conjugated streptavidin. Stained cells were then sorted on a FACSAria cell sorter as shown in FIG. 9. FACS histogram of the first round of sorting revealed that most of the cells stained positive for AAL. The sorting gate for AAL-negative (AAL−ve) cells was set based on unstained CHO cells and this collected the lowest 0.5% of AAL-stained cells.

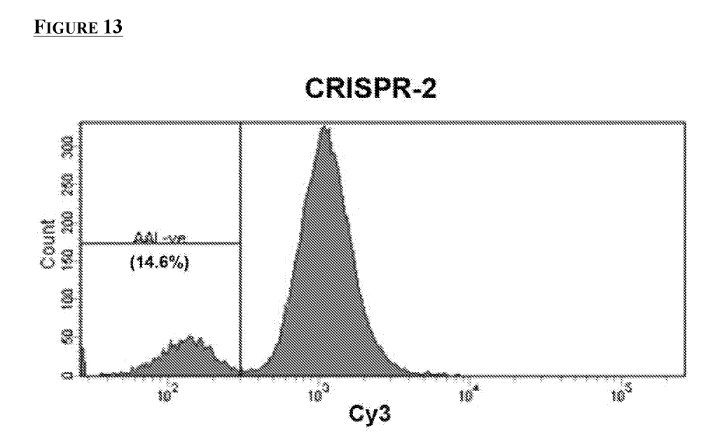

About 1.2×10⁴ cells were collected from 3.5×10⁶ of each transfected cell pool. The collected AAL−ve cells were cultured for approximately 2 weeks and subjected to a second round of sorting. The AAL−ve cells collected from the second round of sorting were cultured and sorted again. For ZFNs-transfected cells, after first round of sorting AAL−ve cell population reached 50% of the total cells. For TALENs- and CRISPR-1-transfected cells, one round of sorting increased the AAL−ve cell population to more than 90% of the total cells (FIG. 9). These results (FIG. 9 and FIG. 13) seem to correlate well with the differences in gene modification activities suggested by the T7E1 mismatch assay (FIG. 8B). Three rounds of sorting yielded a homogeneous population of AAL−ve cells from each transfected pool (FIG. 9). As a control, the same sorting procedure was repeated using untransfected CHO-K1 cells, no increase in AAL−ve population was observed (data not shown). To confirm that the cells with AAL−ve phenotype lacked functional GDP-fucose transporter, mutant pools were transfected with a plasmid encoding human GDP-fucose transporter. AAL analysis of the transfected cells revealed that the GDP-fucose transporter was able to rescue the AAL−ve phenotype generated by the ZFNs, TALENs and CRISPR-1 (FIG. 9).

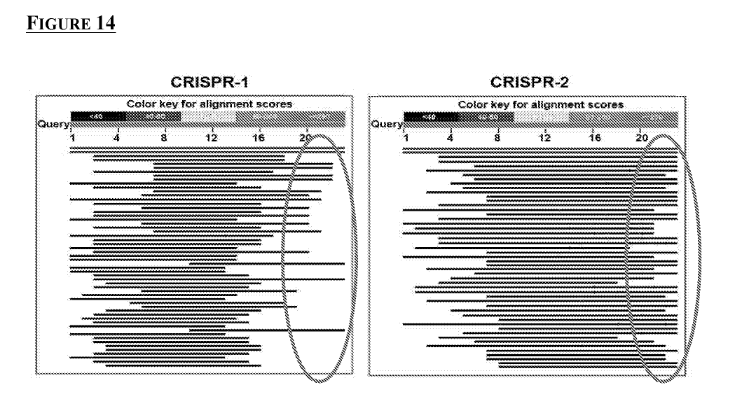

Among the potential CRIPSR target sites that match the GN₂₀GG sequence in the first exon of the coding region, two sites, CRISPR-1 and CRISPR-2, were selected in this work. As shown in FIG. 8B, CRISPR-2 showed highest gene modification activity. It is also more efficient in inactivating the Slc35c1 gene as the first round of FACS showed a significant population of AAL−ve cells (14.6%) (FIG. 13). However, these cells failed to grow and eventually all died. This result could be attributed to the potential off-target effects. A BLAST search against the CHO cell genome suggests that CRISPR-1 shows less sequence matches at the 3′ end (including the PAM sequence) compared to CRISPR-2 (red circles) (FIG. 14), suggesting that CRISPR-1 is likely to have less off-target effects and therefore is more specific [50, 51].

### Example 41

### Inactivation of GDP-Fucose Transporter Gene (Slc35c1) in CHO Cells—Results—Molecular Characterization of the CHO-gmt3 Lines

CHO cells that lack functional GDP-fucose transporter due to mutated Slc35c1 gene have been named CHO-gmt3 cells. To characterize the mutation in different CHO-gmt3 clones at the molecular level, single clones were isolated from the AAL−ve populations. All these single clones stained negatively with AAL and the AAL−ve phenotype was rescued by the human GDP-fucose transporter (data not shown). Genomic DNA from these clones was extracted and the Slc35c1 locus was amplified by PCR. PCR products were then cloned into a TOPO vector and sequenced. The sequencing data revealed deletion or insertion mutations characteristic of NHEJ DNA repair at the respective ZFN, TALEN and CRISPR-1 cleavage sites (Table E1 below).

TABLE E1
Mutations in the GDP-fucose transporter gene in CHO cells generated by
the ZFNs, TALENs and CRISPR-1.
Mutations introduced by the ZFNs:

| | | |
|---|---|---|
| WT | 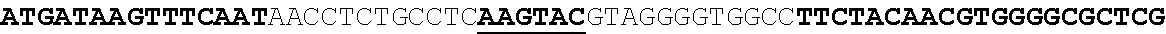 | |
| 1A | 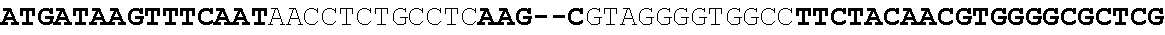 | −2 |
| 1B | 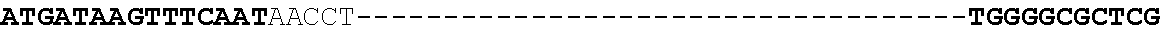 | −35 |
| 2A | 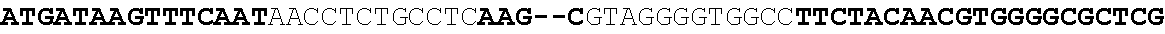 | −2 |
| 2B | 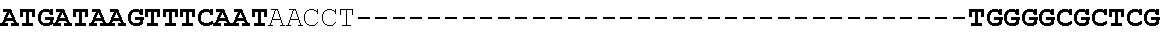 | −35 |
| 3A | 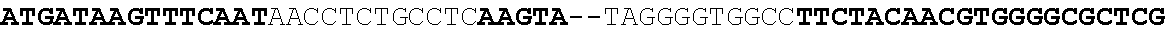 | −2 |
| 3B | 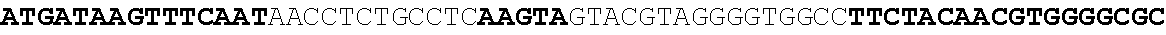 | +3 |
| 4A | 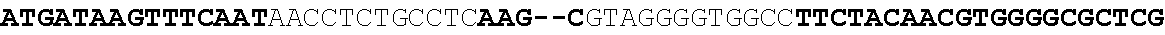 | −2 |
| 4B | 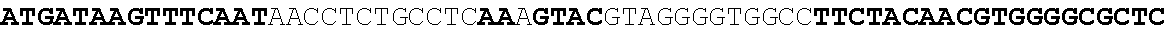 | +1 |
| 5 | 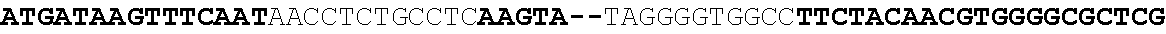 | −2 |
| 6 | 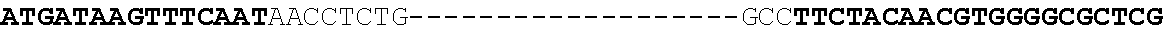 | −19 |
| 7 | 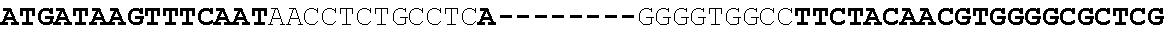 | −8 |
| 8A | 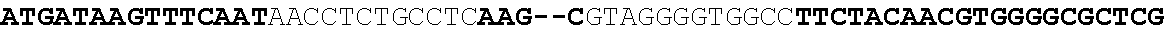 | −2 |
| 8B | 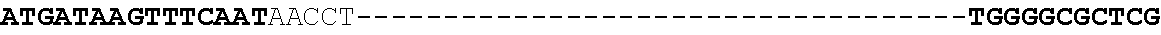 | −35 |
| 9A | 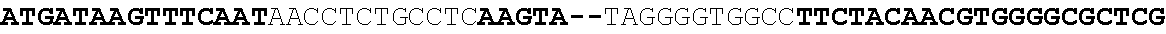 | −2 |
| 9B | 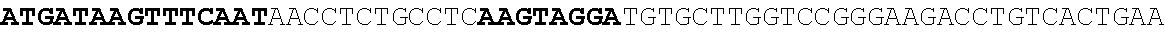 | +108 |
| | 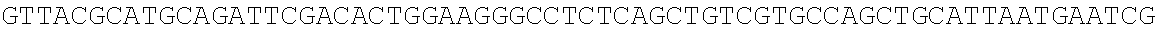 | |
| | 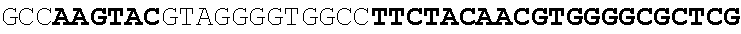 | |

Mutations introduced by the TALENs:

| | | |
|---|---|---|
| WT | 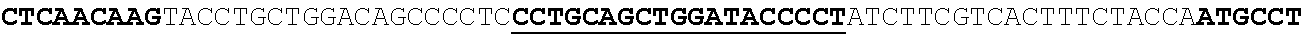 | |
| 1A | 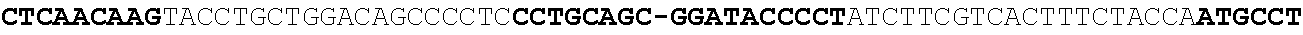 | −1 |
| 1B | 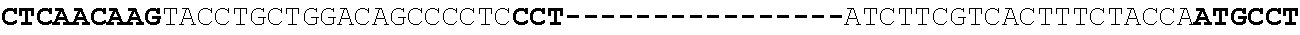 | −16 |
| 2A | 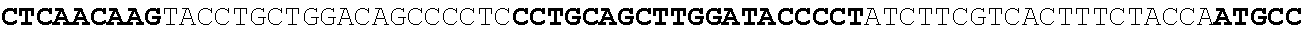 | +1 |
| 2B | 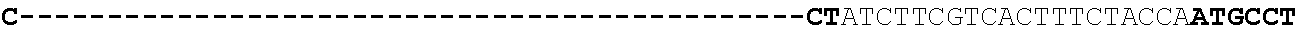 | −45 |
| 3A | 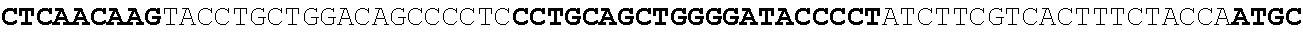 | +2 |
| 3B | 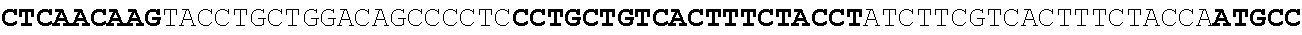 | +1* |
| 4 | 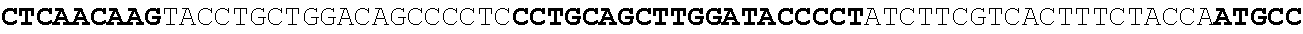 | +1 |
| 5A | 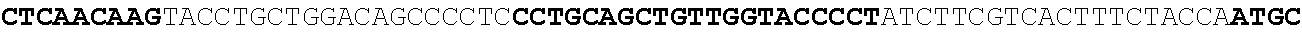 | +2* |
| 5B | 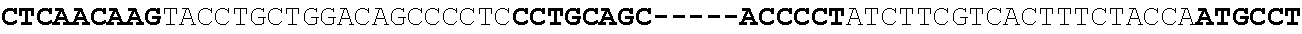 | −5 |
| 6A | 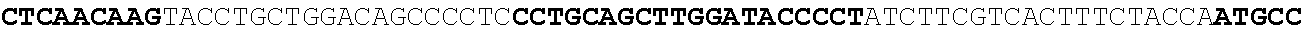 | +1 |
| 6B | 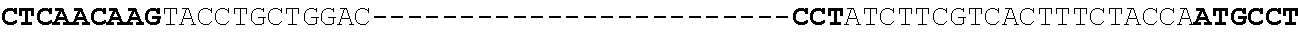 | −24 |
| 7 | 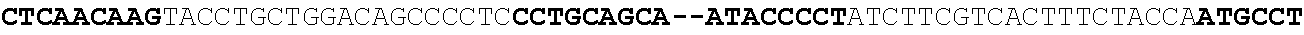 | −2* |
| 8 | 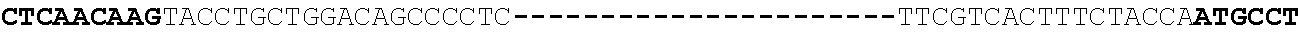 | −22 |
| 9A | 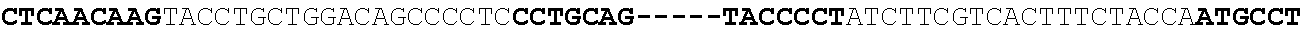 | −5 |
| 9B | 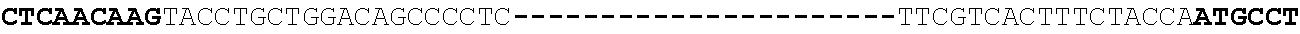 | −22 |

Mutations introduced by CRISPR-1:

| | | |
|---|---|---|
| WT | 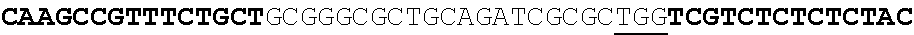 | |
| 1A | 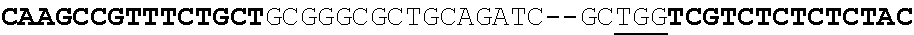 | −2 |
| 1B | 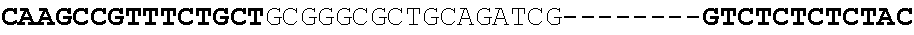 | −8 |
| 2A | 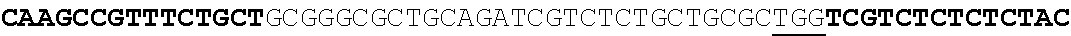 | +9 |
| 2B | 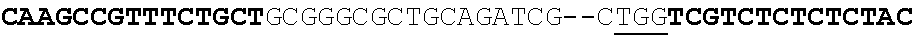 | −2 |
| 3A | 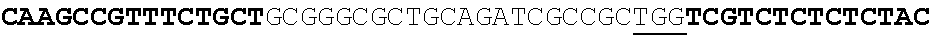 | +1 |
| 3B | 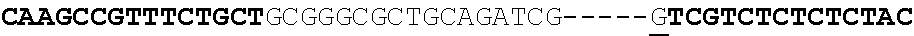 | −5 |
| 4A | 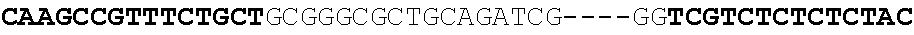 | −4 |
| 4B | 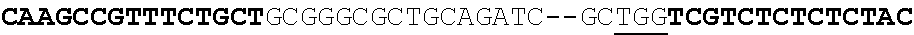 | −2 |
| 5A | 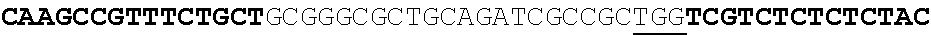 | +1 |
| 5B | 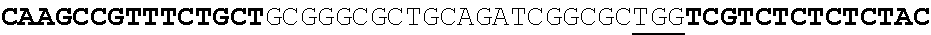 | +1 |
| 6A | 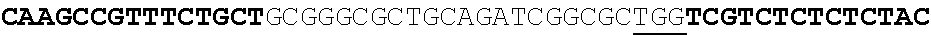 | +1 |
| 6B | 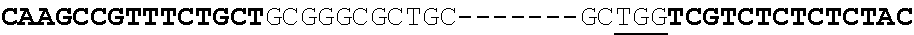 | −7 |
| 7 | 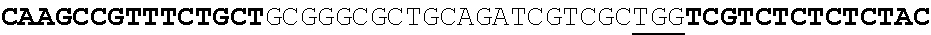 | +1 |
| 8A | 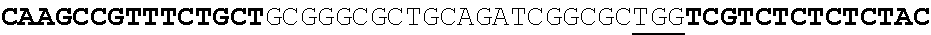 | +1 |
| 8B | 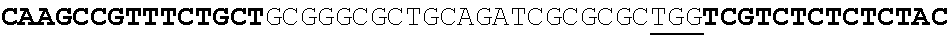 | +2 |
| 8C | 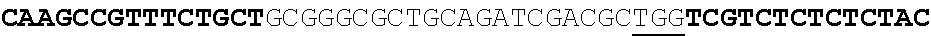 | +1 |

Wild type (WT) sequence for each target site is shown on top. Binding sequences by the ZFNs, TALENs or CRISPR-1 are highlighted in red. Clone names are shown on the left. Deletion mutations (--) or insertion mutations (bases highlighted in blue) are summarized on the right. *partial DNA substitution.

Most clones showed two different mutations, suggesting that both alleles (A and B) of the Slc35c1 gene have been mutated. In some CHO cell clones (ZFNs clones 5, 6 and 7; TALENs clones 4, 7 and 8; CRISPR-1 clone 7), only one type of mutation was observed after 10 bacteria TOPO clones were sequenced. One explanation is that there is only one allele of the Slc35c1 gene in that particular cell. Another possibility could be loss of another mutant allele due to chromosomal translocation between DSBs at on-target site and off-target site [52]. Of course, it is also possible that both alleles happen to have the same mutation. Mutant clones with 3 mutated alleles were also detected (CRISPR-1 clone 8).

### Example 42

### Inactivation of GDP-Fucose Transporter Gene (Slc35c1) in CHO Cells—Results—MALDI-TOF Analysis of N-Glycans Released from Recombinant EPO-Fc Produced by CHO-gmt3 Cells

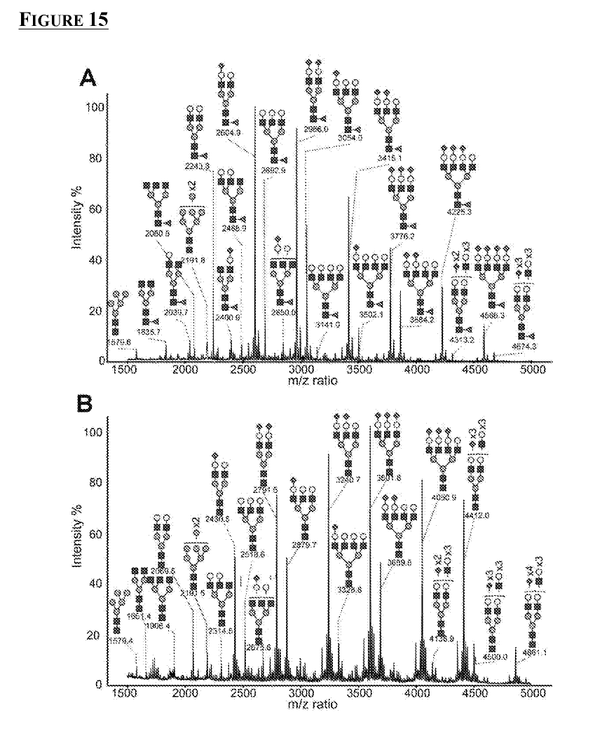

For N-glycan analysis, EPO-Fc fusion protein was produced in wild-type CHO-K1 cells and one ZFN-inactivated CHO-gmt3 mutant clone (ZFNs clone 8 in Table E1 above). Recombinant EPO-Fc in the conditioned medium was purified by a Protein A column. The N-glycans attached to the purified EPO-Fc samples were then released by PNGase F and analyzed by MALDI-TOF MS as previously described [30, 39, 40]. Wild type CHO-K1 cells produced a mixture of mostly core-fucosylated complex N-glycans with bi-, tri-, and tetra-antennary structures sialylated with Neu5Ac residues (FIG. 15A). On the other hand, CHO-gmt3 produced fucose-free complex N-glycans with bi-, tri-, and tetra-antennary structures (FIG. 15B). These data confirmed that the loss of GDP-fucose transporter activity in CHO-gmt3 led to the production of glycoproteins with fucose-free N-glycans.

### Example 43

### Inactivation of GDP-Fucose Transporter Gene (Slc35c1) in CHO Cells—Results—Inactivation of Slc35c1 Gene in a Pre-Existing Anti-Her2 Antibody-Producing CHO Cell Line

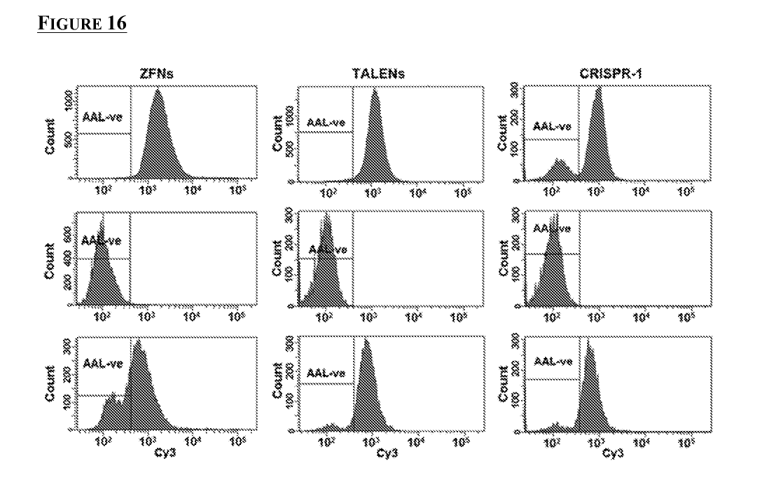

We next illustrate the direct application of the AAL-FACS approach to engineer an existing antibody-producing line to produce fucose-free antibodies. Suspension culture of a pre-existing CHO DG44 cell line, CHO-HER, that produces recombinant anti-Her2 antibody [29] was separately transfected with the same pair of ZFNs, TALENs and CRISPR-1 targeting the Slc35c1 gene. 7-10 days post-transfection, the cells were subjected to the AAL-FACS procedure where the AAL−ve cells were collected and cultured (FIG. 16). Similarly, after 3 rounds of sorting, a homogeneous AAL−ve mutant population was obtained. Restoration of AAL positive (AAL+ve) phenotype upon transfection with the human GDP-fucose transporter cDNA confirms the successful inactivation of GDP-fucose transporter activity with three different technologies in the antibody-producing cell line.

### Example 44

### Inactivation of GDP-Fucose Transporter Gene (Slc35c1) in CHO Cells—Results—Production of Fucose-Free Anti-Her2 Antibodies by the Mutant Cells

The parental CHO-HER and ZFN-inactivated GDP-fucose transporter mutant population CHO-HER (ZFNs) were grown in suspension batch culture in protein-free medium. The anti-Her2 antibody secreted into the medium was then harvested and purified. As a control, we have also included commercial Herceptin for the glycan analysis. N-glycans were released by PNGase F and analyzed by MALDI-TOF MS. Results showed that majority of the N-glycans released from Herceptin and parental CHO-HER-produced anti-Her2 are fucosylated with most abundant species being G0F, G1F and G2F (FIG. 10A and FIG. 10B). In contrast, the N-glycans produced by the mutant populations are fucose-free with majority of the structures being G0, G1 and G2 (FIG. 10C). As an orthogonal analysis, the released N-glycans were also examined by HILIC-UPLC-QTOF experiments. After labeling with 2-AB, the N-glycans were separated and profiled using HILIC-UPLC. Consistent glycan profiles of the samples were observed for both MALDI-TOF and HILIC-UPLC conditions. Similar to the MALDI-TOF MS data, HILIC-UPLC analysis of the N-glycans from Herceptin (FIG. 10D) and parental CHO-HER-produced anti-Her2 (FIG. 10E) revealed that the most abundant N-glycans are G0F, followed by G1F and G2F. The HILIC-UPLC profile of anti-Her2 produced by mutant CHO-HER (FIG. 10F) indicated the absence of fucosylated N-glycans. The amounts of each N-glycan presented as percentage of total N-glycans shown in FIG. 10D, FIG. 10E and FIG. 10F are summarized and compared. The results show that 91.54% of the N-glycans attached to Roche-produced Herceptin are fucosylated, 91.83% of the N-glycans released from the in-house-produced anti-Her2 antibody are fucosylated. The N-glycans produced by the mutant cells are completely lack of fucose (Table E2 below).

TABLE E2
Summary of relative abundance of individual glycans
and glycan groups in Herceptin, parental CHO-HER,
and mutant CHO-HER antibody samples. Differences
in fucosylated glycans were highlighted in bold.

| Glycan abbreviation | | Parental | Mutant |
|---|---|---|---|
| (Oxford) | Herceptin ® | CHO-HER | CHO-HER |
| M3 (%) | — | 0.39 | 0.27 |
| A1 (%) | 0.62 | 0.13 | 0.69 |
| M4 (%) | — | 0.07 | — |
| F(6)A1 (%) | 1.31 | 0.5 | — |
| A2 (%) | 3.42 | 0.16 | 40.26 |
| A1G(4)1 (%) | — | 2.42 | 2.55 |
| F(6)A2 (%) | 47.89 | 16.42 | — |
| M5 (%) | 1.44 | 6.67 | 2.38 |
| A2[6]G(4)1 (%) | 0.89 | — | 32.49 |
| F(6)A1[3]G(4)1 (%) | — | 5.54 | |
| A2[3]G(4)1 (%) | 1.24 | — | 10.84 |
| M5A1 (%) | — | — | 0.34 |
| F(6)A2[6]G(4)1 (%) | 26.8 | 37.61 | — |
| F(6)A2[3]G(4)1 (%) | 9.2 | 8.92 | — |
| M6 (%) | 0.12 | 0.27 | 0.37 |
| F(6)M4A1G(4)1 (%) | — | 0.55 | — |
| A2G(4)2 (%) | 0.48 | 0.26 | 8.49 |
| F(6)A2G(4)2 (%) | 5.5 | 18.08 | — |
| F(6)A1[3]G(4)1S(3)1 (%) | — | 0.11 | |
| M7 D3 (%) | 0.06 | — | — |
| M7 D1 (%) | 0.06 | — | — |
| M7 (%) | — | 0.26 | 0.10 |
| A2G(4)2S(3)1 (%) | — | — | 0.70 |
| F(6)A2G(4)2S(3)1 (%) | 0.57 | 1.1 | — |
| M8 D2,D3 (%) | 0.07 | 0.09 | 0.13 |
| A2G(4)2S(3,3)2 (%) | — | 0.05 | 0.25 |
| M8 D1,D3 (%) | 0.06 | 0.07 | 0.11 |
| F(6)A2G(4)2S(3,3)2 (%) | 0.27 | 0.31 | — |
| M9 (%) | — | — | 0.03 |
| Sialylated Glycans (%) | 0.84 | 1.56 | 1.05 |
| Fucosylated Glycans (%) | 91.54 | 91.83 | 0 |
| High Mannose Glycans (%) | 1.81 | 7.37 | 3.37 |

Glycans not detected were denoted with “—”.

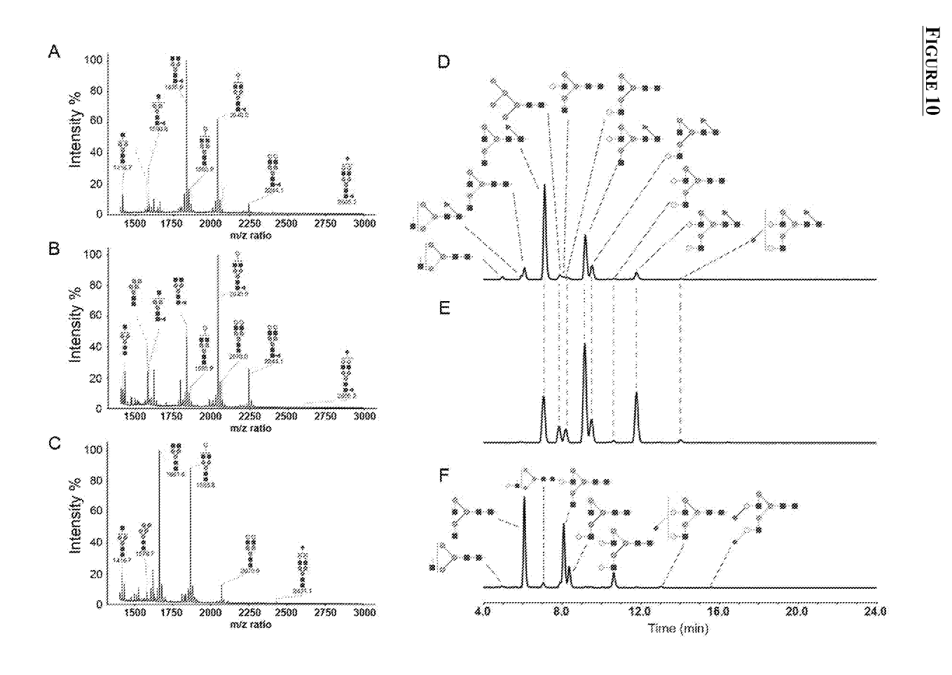

To resolve glycan structure ambiguity, glycans attached to the anti-Her2 antibody produced by the wild type parental CHO-HER cells (FIG. 11A) and the mutant CHO-HER cells (FIG. 11B) were further ‘sequenced’ through digestion with an array of exoglycosidases [53]. These results further confirmed the absence of fucosylation in anti-Her2 produced by mutant CHO-HER. Despite a slightly higher G1F % versus G0F % observed for anti-Her2 produced by parental CHO-HER (which can be attributed to culture conditions), we have shown that antibodies produced by mutant CHO-HER tend to have a similar N-glycan composition (fucose-free form) compared to Herceptin. Collectively, these results support the feasibility of direct inactivation of GDP-fucose transporter with ZFNs, TALENs or CRISPR-Cas9 in antibody-producing CHO cells to generate mutant CHO cells that produce fucose-free antibodies.

### Example 45

### Inactivation of GDP-Fucose Transporter Gene (Slc35c1) in CHO Cells—Results—Growth and Productivity Analysis of GDP-Fucose Transporter-Deficient CHO-HER Cells

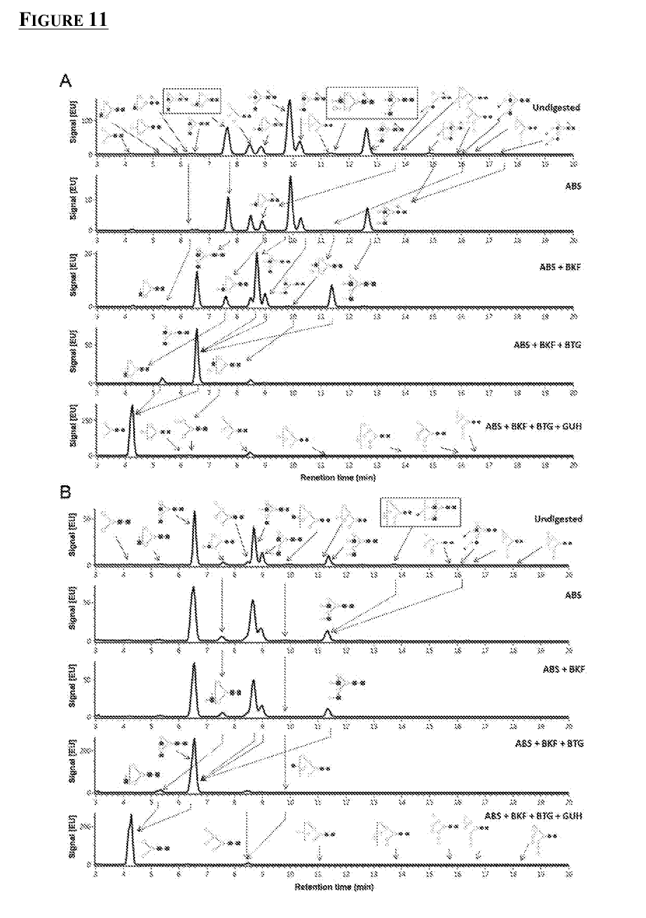

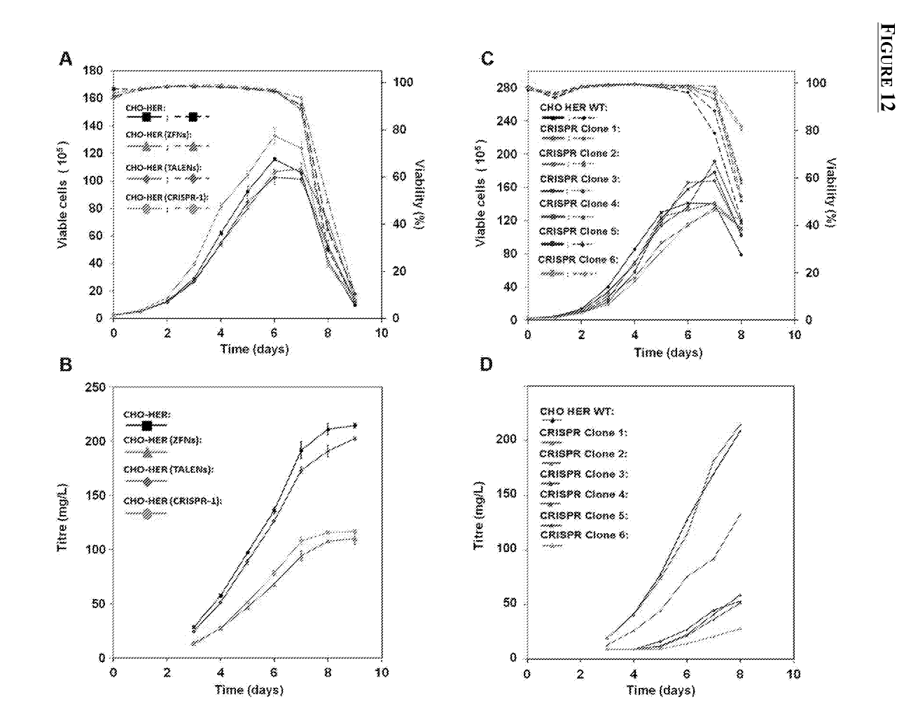

To assess the suitability of the mutant cell lines generated by the ZFNs, TALENs or CRISPR for large-scale bioprocess and manufacturing of fucose-free antibodies, we tested the growth properties and productivity of these mutant lines. Adherent CHO-gmt3 mutant was adapted to protein-free suspension shake flask culture and we observed a growth pattern that was comparable to suspension wild-type CHO-K1 cells (data not shown). Growth pattern of CHO-HER and different mutant CHO-HER populations were also examined. Viable cell density and percentage viability of batch cultures of CHO-HER, CHO-HER (ZFNs), CHO-HER (TALENs) and CHO-HER (CRISPR-1) were measured over a course of 9 days and results were shown (FIG. 12A). CHO-HER (ZFNs), CHO-HER (TALENs) and CHO-HER (CRISPR-1) mutant pools exhibited comparable growth characteristics to their parental cell line CHO-HER with similar logarithmic, stationary and death phases.

In 2-L fed-batch bioreactor runs, the antibody productivity by the parental CHO-HER cells are constantly higher than 2 g/L (unpublished data). The amounts of antibodies produced in the media in the same set of shake flask batch cultures shown in FIG. 12A were determined at different time points from day 3 to day 9 (FIG. 12B). All three mutant populations shared a very similar growth profile to its parental CHO-HER cells (FIG. 12A), which demonstrates that inactivation of Slc35c1 does not affect cells growth. The amounts of antibody produced by three mutant pools were compared with the parental CHO-HER cells (FIG. 12B). CHO-HER (TALENs) population showed similar antibody titer to the parental CHO-HER cells, suggesting that inactivation of the Slc35c1 gene does not affect antibody productivity in CHO cells. The titers for CHO-HER (ZFNs) and CHO-HER (CRISPR-1) mutant pools, however, were lower compared to the parental line.

To address this issue, we randomly picked 6 single clones from the CHO-HER (CRISPR-1) mutant pool and a similar shake flask batch culture experiment was carried out. The growth profiles of the parental CHO-HER cells and the 6 CHO-HER (CRISPR-1) clones were similar (FIG. 12C). However, only Clone 1 exhibited a similar productivity as the parental CHO-HER cells, whereas others showed reduced productivities (FIG. 12D). Whether the reduced productivities observed in these pools is due to off-target effects remains to be investigated. Nonetheless, high producing clones can be isolated from the mutant pools generated by the CRISPR-Cas9 technology. The same may be true for the ZFNs-generated pool.

### Example 46

### Inactivation of GDP-Fucose Transporter Gene (Slc35c1) in CHO Cells—Discussion

In mammalian cells, two pathways lead to the production of GDP-fucose, namely the de novo pathway and the salvage pathway. CHO Lec13 cells have a mutation in the GDP-mannose dehydratase gene in the de novo pathway that reduces the amount of GDP-fucose in the cell but does not completely eliminate core fucosylation [11]. A total of thirteen fucosyltransferases have been characterized, only FUT8 possesses α1,6-transferase activity with the ability to transfer fucose to the core position on N-glycans [12]. Inactivation of Fut8 gene has been shown to prevent core fucosylation in CHO cells [35]. It is known that in wild-type CHO cells, core fucose is the only fucose present on the N-glycans [42]. The exceptions are CHO cell mutants LEC11 and LEC12 that carry novel gain-of-function mutations and express sialyl Lewis^{X} epitope [54]. No fucose is found on the shortened mucin type O-glycans [42]. The CHO-K1 transcriptome data have shown that among all fucosyltransferases, only FUT8 and two protein O-fucosyltransferases (POFUT-1 and POFUT-2) are expressed [55]. As both POFUT-1 and POFUT-2 are localized in the ER [56,57], protein O-fucosylation by POFUT-1 and POFUT-2 will not be affected as SLC35C1 is only responsible for transporting GDP-fucose into the Golgi. Therefore, the only fucosyltransferase that should be affected by inactivating the Golgi GDP-fucose transporter gene (Slc35c1) is FUT8. As such, inactivating Fut8 or Slc35c1 should have similar impact on CHO-K1 cells. A potential advantage of knocking out Slc35c1 over Fut8 is that it eliminates the potential complications caused by the gain-of-function mutations found in LEC11 and LEC12 cells [54]. Our results have shown that inactivation of Slc35c1 gene in CHO cells did not seem to affect cell growth, viable cell density and antibody productivity. Data presented in this report suggest that inactivation of GDP-fucose transporter in CHO cells represents a feasible strategy for production of fucose-free antibodies.

Three different genome-editing technologies were used to inactivate GDP-fucose transporter in CHO cells. For ZFNs, based on data from published literature, we have assembled a pair of ZFNs to target GDP-fucose transporter. Essentially, the array of zinc-finger units can be re-assembled using different finger units to target different DNA sequences by the modular assembly method [58]. However, the ZFNs design is often complicated by context dependent effects of zinc-finger units arising from crosstalk between zinc-fingers and overlap effects of 3-bp target sites. Engineering and screening for a ZFN pair with high binding affinity and specificity to target a given DNA sequence require special expertise and resources.

TALENs represent a more modular mode of DNA recognition. The one-to-one correspondence between the RVDs and target nucleotides means that site-specific TALEs can be designed by having the linear array of TALE repeats matching to target DNA sequence in the 5′ to 3′ direction. Various methods to assemble TALENs have been reported [34, 35, 59, 60] and we found the Golden-Gate methodology to be a relatively easy method. The main disadvantage with TALENs is that it is still not clearly understood why certain assembled TALENs work and others do not.

The major advantage of the CRISPR-Cas9 system lies in the easy design of gRNAs and high gene modification efficiency. Unlike ZFNs and TALENs that rely on protein-DNA interactions to recognize target sites in the chromosome, CRISPR-Cas9 system recognizes target sites by Watson-Crick base pairing between gRNA and DNA sequence. Multiple gRNAs can be introduced simultaneously to enable editing of multiple genes in the same cell [26, 28]. A major caveat with the use of CRISPR system is that it tolerates base pair mismatches between gRNA and its complementary target sequence primarily at the 5′ end >12-bp from the PAM sequence [26] and up to 6-bp mismatches at the target site [27]. However, this can be circumvented by the use of nickases in the form of mutant Cas9 to generate targeted mutagenesis in the presence of donor template by HDR to reduce off-target effects [26, 27].

Although the CRISPR-Cas9 technology is easy to use for cell line engineering, potential off-target effect remains to be a potential problem. As we have shown in this study, antibody productivity of the mutant pool generated by TALENs is comparable to that of the parental cells, whereas the productivity of the mutant pools generated by ZFNs and CRISPR-1 decreased, despite all having similar growth profiles to the parental CHO-HER cell line. Whether the reduced antibody productivity in CHO-HER (ZFNs) and CHO-HER (CRISPR-1) mutant pools was due to off-target effect are being investigated.

The use of FACS approach allows us to enrich for genetically-engineered mutants with the desired phenotype. This is in contrast to previous strategies of identifying genotype which can be time-consuming. Importantly, fucose-free antibody-producing cell lines can be rapidly generated from pre-existing antibody-producing lines in less than two months. We have also demonstrated that the FACS approach is effective in isolating mutants engineered by custom nucleases having low gene modification activity. Apart from lectins, we believe that antibodies specific for certain cell surface antigens can also be used in this FACS approach to select for ZFN/TALEN/CRISPR-engineered cells.

### REFERENCES

Ackerman M E, Crispin M, Yu X, Baruah K, Boesch A W, Harvey D J, Dugast A S, Heizen E L, Ercan A, Choi I and others. 2013. Natural variation in Fc glycosylation of HIV-specific antibodies impacts antiviral activity. J Clin Invest 123(5):2183-92.

Cardarelli P M, Moldovan-Loomis M C, Preston B, Black A, Passmore D, Chen T H, Chen S, Liu J, Kuhne M R, Srinivasan M and others. 2009. In vitro and in vivo characterization of MDX-1401 for therapy of malignant lymphoma. Clin Cancer Res 15(10):3376-83.

Conradt H S, Egge H, Peter-Katalinic J, Reiser W, Siklosi T, Schaper K. 1987. Structure of the carbohydrate moiety of human interferon-beta secreted by a recombinant Chinese hamster ovary cell line. J Biol Chem 262(30):14600-5.

Costa A R, Withers J, Rodrigues M E, McLoughlin N, Henriques M, Oliveira R, Rudd P M, Azeredo J. 2013. The impact of cell adaptation to serum-free conditions on the glycosylation profile of a monoclonal antibody produced by Chinese hamster ovary cells. N Biotechnol 30(5):563-72.

Davies J, Jiang L, Pan L Z, LaBarre M J, Anderson D, Reff M. 2001. Expression of GnTIII in a recombinant anti-CD20 CHO production cell line: Expression of antibodies with altered glycoforms leads to an increase in ADCC through higher affinity for FC gamma RIII. Biotechnol Bioeng 74(4):288-94.

Deutscher S L, Hirschberg C B. 1986. Mechanism of galactosylation in the Golgi apparatus. A Chinese hamster ovary cell mutant deficient in translocation of UDP-galactose across Golgi vesicle membranes. J Biol Chem 261(1):96-100.

Goh J S, Zhang P, Chan K F, Lee M M, Lim S F, Song Z. 2010. RCA-I-resistant CHO mutant cells have dysfunctional GnT I and expression of normal GnT I in these mutants enhances sialylation of recombinant erythropoietin. Metab Eng 12(4):360-8.

Haryadi R, Zhang P, Chan K F, Song Z. 2013. CHO-gmt5, a novel CHO glycosylation mutant for producing afucosylated and asialylated recombinant antibodies. Bioengineered 4(2):90-4.

Ho S C, Bardor M, Feng H, Mariati, Tong Y W, Song Z, Yap M G, Yang Y. 2012. IRES-mediated Tricistronic vectors for enhancing generation of high monoclonal antibody expressing CHO cell lines. J Biotechnol 157(1):130-9.

Hristodorov D, Fischer R, Linden L. 2013. With or without sugar? (A) glycosylation of therapeutic antibodies. Mol Biotechnol 54(3):1056-68.

Ito K, Furukawa J, Yamada K, Tran N L, Shinohara Y, Izui S. 2014. Lack of galactosylation enhances the pathogenic activity of IgG1 but Not IgG2a anti-erythrocyte autoantibodies. J Immunol 192(2):581-8.

Jung S T, Kang T H, Kelton W, Georgiou G. 2011. Bypassing glycosylation: engineering aglycosylated full-length IgG antibodies for human therapy. Curr Opin Biotechnol 22(6):858-67.

Junttila T T, Parsons K, Olsson C, Lu Y, Xin Y, Theriault J, Crocker L, Pabonan O, Baginski T, Meng G and others. 2010. Superior in vivo efficacy of afucosylated trastuzumab in the treatment of HER2-amplified breast cancer. Cancer Res 70(11):4481-9.

Knibbs R N, Goldstein I J, Ratcliffe R M, Shibuya N. 1991. Characterization of the carbohydrate binding specificity of the leukoagglutinating lectin from Maackia amurensis. Comparison with other sialic acid-specific lectins. J Biol Chem 266(1):83-8.

Krapp S, Mimura Y, Jefferis R, Huber R, Sondermann P. 2003. Structural analysis of human IgG-Fc glycoforms reveals a correlation between glycosylation and structural integrity. J Mol Biol 325(5):979-89.

Lim S F, Lee M M, Zhang P, Song Z. 2008. The Golgi CMP-sialic acid transporter: A new CHO mutant provides functional insights. Glycobiology 18(11):851-60.

Loh W P, Loo B, Zhou L, Zhang P, Lee D Y, Yang Y, Lam K P. 2014. Overexpression of microRNAs enhances recombinant protein production in Chinese hamster ovary cells. Biotechnol J 9(9):1140-51.

Matsumiya S, Yamaguchi Y, Saito J, Nagano M, Sasakawa H, Otaki S, Satoh M, Shitara K, Kato K. 2007. Structural comparison of fucosylated and nonfucosylated Fc fragments of human immunoglobulin G1. J Mol Biol 368(3):767-79.

Matsumura K, Higashida K, Ishida H, Hata Y, Yamamoto K, Shigeta M, Mizuno-Horikawa Y, Wang X, Miyoshi E, Gu J and others. 2007. Carbohydrate binding specificity of a fucose-specific lectin from Aspergillus oryzae: a novel probe for core fucose. J Biol Chem 282(21):15700-8.

Niwa R, Shoji-Hosaka E, Sakurada M, Shinkawa T, Uchida K, Nakamura K, Matsushima K, Ueda R, Hanai N, Shitara K. 2004. Defucosylated chimeric anti-CC chemokine receptor 4 IgG1 with enhanced antibody-dependent cellular cytotoxicity shows potent therapeutic activity to T-cell leukemia and lymphoma. Cancer Res 64(6):2127-33.

Oelmann S, Stanley P, Gerardy-Schahn R. 2001. Point mutations identified in Lec8 Chinese hamster ovary glycosylation mutants that inactivate both the UDP-galactose and CMP-sialic acid transporters. J Biol Chem 276(28):26291-300.

Okazaki A, Shoji-Hosaka E, Nakamura K, Wakitani M, Uchida K, Kakita S, Tsumoto K, Kumagai I, Shitara K. 2004. Fucose depletion from human IgG1 oligosaccharide enhances binding enthalpy and association rate between IgG1 and FcgammaRIIIa. J Mol Biol 336(5):1239-49.

Quast I, Lunemann J D. 2014. Fc glycan-modulated immunoglobulin g effector functions. J Clin Immunol 34 Suppl 1:51-5.

Radaev S, Motyka S, Fridman W H, Sautes-Fridman C, Sun P D. 2001. The structure of a human type III Fcgamma receptor in complex with Fc. J Biol Chem 276(19):16469-77.

Radaev S, Sun P. 2002. Recognition of immunoglobulins by Fcgamma receptors. Mol Immunol 38(14):1073-83.

Schiestl M, Stangler T, Torella C, Cepeljnik T, Toll H, Grau R. 2011. Acceptable changes in quality attributes of glycosylated biopharmaceuticals. Nat Biotechnol 29(4):310-2.

Schriebl K, Trummer E, Weik R, Muller D, Kunert R, Lattenmayer C, Katinger H, Vorauer-Uhl K. 2006. A novel strategy for quantitative isoform detection directly performed from culture supernatant. J Pharm Biomed Anal 42(3):322-7.

Shields R L, Lai J, Keck R, O'Connell L Y, Hong K, Meng Y G, Weikert S H, Presta L G. 2002. Lack of fucose on human IgG1 N-linked oligosaccharide improves binding to human Fcgamma RIII and antibody-dependent cellular toxicity. J Biol Chem 277(30):26733-40.

Shinkawa T, Nakamura K, Yamane N, Shoji-Hosaka E, Kanda Y, Sakurada M, Uchida K, Anazawa H, Satoh M, Yamasaki M and others. 2003. The absence of fucose but not the presence of galactose or bisecting N-acetylglucosamine of human IgG1 complex-type oligosaccharides shows the critical role of enhancing antibody-dependent cellular cytotoxicity. J Biol Chem 278(5):3466-73.

Sondermann P, Huber R, Oosthuizen V, Jacob U. 2000. The 3.2-A crystal structure of the human IgG1 Fc fragment-Fc gammaRIII complex. Nature 406(6793):267-73.

Suzuki E, Niwa R, Saji S, Muta M, Hirose M, Iida S, Shiotsu Y, Satoh M, Shitara K, Kondo M and others. 2007. A nonfucosylated anti-HER2 antibody augments antibody-dependent cellular cytotoxicity in breast cancer patients. Clin Cancer Res 13(6):1875-82.

van Berkel P H, Gerritsen J, Perdok G, Valbjorn J, Vink T, van de Winkel J G, Parren P W. 2009. N-linked glycosylation is an important parameter for optimal selection of cell lines producing biopharmaceutical human IgG. Biotechnol Prog 25(1):244-51.

Wang W C, Cummings R D. 1988. The immobilized leukoagglutinin from the seeds of Maackia amurensis binds with high affinity to complex-type Asn-linked oligosaccharides containing terminal sialic acid-linked alpha-2,3 to penultimate galactose residues. J Biol Chem 263(10):4576-85.

Zhang P, Haryadi R, Chan K F, Teo G, Goh J, Pereira N A, Feng H, Song Z. 2012. Identification of functional elements of the GDP-fucose transporter SLC35C1 using a novel Chinese hamster ovary mutant. Glycobiology 22(7):897-911.

Yamane-Ohnuki N, Kinoshita S, Inoue-Urakubo M, Kusunoki M, Iida S, Nakano R, Wakitani M, Niwa R, Sakurada M, Uchida K, Shitara K, Satoh M. 2004 Establishment of FUT8 knockout Chinese hamster ovary cells: an ideal host cell line for producing completely defucosylated antibodies with enhanced antibody-dependent cellular cytotoxicity. Biotechnol Bioeng. 87(5):614-22.

Becker D J, Lowe J B. 2003 Fucose: biosynthesis and biological function in mammals. Glycobiology. 13(7):41R-53R.

Lu L, Hou X, Shi S, Korner C, Stanley P. 2010 Slc35c2 promotes Notchl fucosylation and is required for optimal Notch signaling in mammalian cells. J Biol Chem. 285(46):36245-54.

Loriol C, Dupuy F, Rampal R, Dlugosz M A, Haltiwanger R S, Maftah A, Germot A. 2006 Molecular evolution of protein O-fucosyltransferase genes and splice variants. Glycobiology. 16(8):736-47.

### REFERENCES FOR EXAMPLES 19 TO 46

[1] Radaev, S., Motyka, S., Fridman, W. H., Sautes-Fridman, C., Sun, P. D., The structure of a human type III Fcgamma receptor in complex with Fc. J. Biol. Chem. 2001, 276, 16469-16477.

[2] Sondermann, P., Huber, R., Oosthuizen, V., Jacob, U., The 3.2-A crystal structure of the human IgG1 Fc fragment-Fc gammaRIII complex. Nature 2000, 406, 267-273.

[3] Krapp, S., Mimura, Y., Jefferis, R., Huber, R., Sondermann, P., Structural analysis of human IgG-Fc glycoforms reveals a correlation between glycosylation and structural integrity. J. Mol. Biol. 2003, 325, 979-989.

[4] Shields, R. L., Lai, J., Keck, R., O'Connell, L. Y., et al., Lack of fucose on human IgG1 N-linked oligosaccharide improves binding to human Fcgamma RIII and antibody-dependent cellular toxicity. J. Biol. Chem. 2002, 277, 26733-26740.

[5] Shinkawa, T., Nakamura, K., Yamane, N., Shoji-Hosaka, E., et al., The absence of fucose but not the presence of galactose or bisecting N-acetylglucosamine of human IgG1 complex-type oligosaccharides shows the critical role of enhancing antibody-dependent cellular cytotoxicity. J. Biol. Chem. 2003, 278, 3466-3473.

[6] Cardarelli, P. M., Moldovan-Loomis, M. C., Preston, B., Black, A., et al., In vitro and in vivo characterization of MDX-1401 for therapy of malignant lymphoma. Clin. Cancer Res. 2009, 15, 3376-3383.

[7] Junttila, T. T., Parsons, K., Olsson, C., Lu, Y., et al., Superior in vivo efficacy of afucosylated trastuzumab in the treatment of HER2-amplified breast cancer. Cancer Res. 2010, 70, 4481-4489.

[8] Niwa, R., Hatanaka, S., Shoji-Hosaka, E., Sakurada, M., et al., Enhancement of the antibody-dependent cellular cytotoxicity of low-fucose IgG1 Is independent of FcgammaRIIIa functional polymorphism. Clin. Cancer Res. 2004, 10, 6248-6255.

[9] Suzuki, E., Niwa, R., Saji, S., Muta, M., et al., A nonfucosylated anti-HER2 antibody augments antibody-dependent cellular cytotoxicity in breast cancer patients. Clin. Cancer Res. 2007, 13, 1875-1882.

[10] Okeley, N. M., Alley, S. C., Anderson, M. E., Boursalian, T. E., et al., Development of orally active inhibitors of protein and cellular fucosylation. Proc Natl Acad Sci USA. 2013, 110, 5404-5409.

[11] Ohyama, C., Smith, P. L., Angata, K., Fukuda, M. N., et al., Molecular cloning and expression of GDP-D-mannose-4,6-dehydratase, a key enzyme for fucose metabolism defective in Lec13 cells. J. Biol. Chem. 1998, 273, 14582-14587.

[12] Becker, D. J., Lowe, J. B., Fucose: biosynthesis and biological function in mammals. Glycobiology 2003, 13, 41R-53R.

[13] Yamane-Ohnuki, N., Kinoshita, S., Inoue-Urakubo, M., Kusunoki, M., et al., Establishment of FUT8 knockout Chinese hamster ovary cells: an ideal host cell line for producing completely defucosylated antibodies with enhanced antibody-dependent cellular cytotoxicity. Biotechnol. Bioeng. 2004, 87, 614-622.

[14] Malphettes, L., Freyvert, Y., Chang, J., Liu, P. Q., et al., Highly efficient deletion of FUT8 in CHO cell lines using zinc-finger nucleases yields cells that produce completely nonfucosylated antibodies. Biotechnol. Bioeng. 2010, 106, 774-783.

[15] Lu, L., Hou, X., Shi, S., Korner, C., Stanley, P., Slc35c2 promotes Notch1 fucosylation and is required for optimal Notch signaling in mammalian cells. J. Biol. Chem. 2010, 285, 36245-36254.

[16] Lubke, T., Marquardt, T., Etzioni, A., Hartmann, E., et al., Complementation cloning identifies CDG-IIc, a new type of congenital disorders of glycosylation, as a GDP-fucose transporter deficiency. Nat. Genet. 2001, 28, 73-76.

[17] Luhn, K., Wild, M. K., Eckhardt, M., Gerardy-Schahn, R., Vestweber, D., The gene defective in leukocyte adhesion deficiency II encodes a putative GDP-fucose transporter. Nat. Genet. 2001, 28, 69-72.

[18] Doyon, Y., McCammon, J. M., Miller, J. C., Faraji, F., et al., Heritable targeted gene disruption in zebrafish using designed zinc-finger nucleases. Nat. Biotechnol. 2008, 26, 702-708.

[19] Miller, J. C., Holmes, M. C., Wang, J., Guschin, D. Y., et al., An improved zinc-finger nuclease architecture for highly specific genome editing. Nat. Biotechnol. 2007, 25, 778-785.

[20] Urnov, F. D., Miller, J. C., Lee, Y. L., Beausej our, C. M., et al., Highly efficient endogenous human gene correction using designed zinc-finger nucleases. Nature 2005, 435, 646-651.

[21] Christian, M., Cermak, T., Doyle, E. L., Schmidt, C., et al., Targeting DNA double-strand breaks with TAL effector nucleases. Genetics 2010, 186, 757-761.

[22] Miller, J. C., Tan, S., Qiao, G., Barlow, K. A., et al., A TALE nuclease architecture for efficient genome editing. Nat. Biotechnol. 2011, 29, 143-148.

[23] Boch, J., Scholze, H., Schornack, S., Landgraf, A., et al., Breaking the code of DNA binding specificity of TAL-type III effectors. Science 2009, 326, 1509-1512.

[24] Moscou, M. J., Bogdanove, A. J., A simple cipher governs DNA recognition by TAL effectors. Science 2009, 326, 1501.

[25] Kim, Y., Kweon, J., Kim, A., Chon, J. K., et al., A library of TAL effector nucleases spanning the human genome. Nat. Biotechnol. 2013, 31, 251-258.

[26] Cong, L., Ran, F. A., Cox, D., Lin, S., et al., Multiplex genome engineering using CRISPR/Cas systems. Science 2013, 339, 819-823.

[27] Jinek, M., Chylinski, K., Fonfara, I., Hauer, M., et al., A programmable dual-RNA-guided DNA endonuclease in adaptive bacterial immunity. Science 2012, 337, 816-821.

[28] Mali, P., Yang, L., Esvelt, K. M., Aach, J., et al., RNA-guided human genome engineering via Cas9. Science 2013, 339, 823-826.

[29] Ho, S. C., Bardor, M., Feng, H., Mariati, et al., IRES-mediated Tricistronic vectors for enhancing generation of high monoclonal antibody expressing CHO cell lines. J Biotechnol 2012, 157, 130-139.

[30] Zhang, P., Haryadi, R., Chan, K. F., Teo, G., et al., Identification of functional elements of the GDP-fucose transporter SLC35C1 using a novel Chinese hamster ovary mutant. Glycobiology 2012, 22, 897-911.

[31] Bonas, U., Stall, R. E., Staskawicz, B., Genetic and structural characterization of the avirulence gene avrBs3 from Xanthomonas campestris pv. vesicatoria. Mol. Gen. Genet. 1989, 218, 127-136.

[32] Guo, J., Gaj, T., Barbas, C. F., 3rd, Directed evolution of an enhanced and highly efficient FokI cleavage domain for zinc finger nucleases. J. Mol. Biol. 2010, 400, 96-107.

[33] Doyon, Y., Vo, T. D., Mendel, M. C., Greenberg, S. G., et al., Enhancing zinc-finger-nuclease activity with improved obligate heterodimeric architectures. Nat. Methods 2011, 8, 74-79.

[34] Sanjana, N. E., Cong, L., Zhou, Y., Cunniff, M. M., et al., A transcription activator-like effector toolbox for genome engineering. Nat Protoc 2012, 7, 171-192.

[35] Cermak, T., Doyle, E. L., Christian, M., Wang, L., et al., Efficient design and assembly of custom TALEN and other TAL effector-based constructs for DNA targeting. Nucleic Acids Res. 2011, 39, e82.

[36] Doyle, E. L., Booher, N. J., Standage, D. S., Voytas, D. F., et al., TAL Effector-Nucleotide Targeter (TALE-NT) 2.0: tools for TAL effector design and target prediction. Nucleic Acids Res. 2012, 40, W117-122.

[37] Kim, H. J., Lee, H. J., Kim, H., Cho, S. W., Kim, J. S., Targeted genome editing in human cells with zinc finger nucleases constructed via modular assembly. Genome Res. 2009, 19, 1279-1288.

[38] Guschin, D. Y., Waite, A. J., Katibah, G. E., Miller, J. C., et al., A rapid and general assay for monitoring endogenous gene modification. Methods Mol Biol. 2010, 649, 247-256.

[39] Goh, J. S., Zhang, P., Chan, K. F., Lee, M. M., et al., RCA-I-resistant CHO mutant cells have dysfunctional GnT I and expression of normal GnT I in these mutants enhances sialylation of recombinant erythropoietin. Metab Eng. 2010, 12, 360-368.

[40] Lim, S. F., Lee, M. M., Zhang, P., Song, Z., The Golgi CMP-sialic acid transporter: A new CHO mutant provides functional insights. Glycobiology 2008, 18, 851-860.

[41] Dell, A., Reason, A. J., Khoo, K. H., Panico, M., et al., Mass spectrometry of carbohydrate-containing biopolymers. Methods Enzymol. 1994, 230, 108-132.

[42] North, S. J., Huang, H. H., Sundaram, S., Jang-Lee, J., et al., Glycomics profiling of Chinese hamster ovary cell glycosylation mutants reveals N-glycans of a novel size and complexity. J. Biol. Chem. 2010, 285, 5759-5775.

[43] Shahrokh, Z., Royle, L., Saldova, R., Bones, J., et al., Erythropoietin produced in a human cell line (Dynepo) has significant differences in glycosylation compared with erythropoietins produced in CHO cell lines. Mol. Pharm. 2011, 8, 286-296.

[44] Dreier, B., Segal, D. J., Barbas, C. F., 3rd, Insights into the molecular recognition of the 5′-GNN-3′ family of DNA sequences by zinc finger domains. J. Mol. Biol. 2000, 303, 489-502.

[45] Liu, Q., Xia, Z., Zhong, X., Case, C. C., Validated zinc finger protein designs for all 16 GNN DNA triplet targets. J. Biol. Chem. 2002, 277, 3850-3856.

[46] Segal, D. J., Dreier, B., Beerli, R. R., Barbas, C. F., 3rd, Toward controlling gene expression at will: selection and design of zinc finger domains recognizing each of the 5′-GNN-3′ DNA target sequences. Proc Natl Acad Sci USA. 1999, 96, 2758-2763.

[47] Sander, J. D., Zaback, P., Joung, J. K., Voytas, D. F., Dobbs, D., Zinc Finger Targeter (ZiFiT): an engineered zinc finger/target site design tool. Nucleic Acids Res. 2007, 35, W599-605.

[48] Cong, L., Zhou, R., Kuo, Y. C., Cunniff, M., Zhang, F., Comprehensive interrogation of natural TALE DNA-binding modules and transcriptional repressor domains. Nat. Commun. 2012, 3, 968.

[49] Matsumura, K., Higashida, K., Ishida, H., Hata, Y., et al., Carbohydrate binding specificity of a fucose-specific lectin from Aspergillus oryzae: a novel probe for core fucose. J. Biol. Chem. 2007, 282, 15700-15708.

[50] Kuscu, C., Arslan, S., Singh, R., Thorpe, J., Adli, M., Genome-wide analysis reveals characteristics of off-target sites bound by the Cas9 endonuclease. Nat. Biotechnol. 2014, 32, 677-683.

[51] Wu, X., Scott, D. A., Kriz, A. J., Chiu, A. C., et al., Genome-wide binding of the CRISPR endonuclease Cas9 in mammalian cells. Nat. Biotechnol. 2014, 32, 670-676.

[52] Frock, R. L., Hu, J., Meyers, R. M., Ho, Y. J., et al., Genome-wide detection of DNA double-stranded breaks induced by engineered nucleases. Nat. Biotechnol. 2015, 33, 179-186.

[53] Marino, K., Bones, J., Kattla, J. J., Rudd, P. M., A systematic approach to protein glycosylation analysis: a path through the maze. Nat. Chem. Biol. 2010, 6, 713-723.

[54] Howard, D. R., Fukuda, M., Fukuda, M. N., Stanley, P., The GDP-fucose:N-acetylglucosaminide 3-alpha-L-fucosyltransferases of LEC11 and LEC12 Chinese hamster ovary mutants exhibit novel specificities for glycolipid substrates. J. Biol. Chem. 1987, 262, 16830-16837.

[55] Xu, X., Nagaraj an, H., Lewis, N. E., Pan, S., et al., The genomic sequence of the Chinese hamster ovary (CHO)-K1 cell line. Nat. Biotechnol. 2011, 29, 735-741.

[56] Luo, Y., Haltiwanger, R. S., 0-fucosylation of notch occurs in the endoplasmic reticulum. J. Biol. Chem. 2005, 280, 11289-11294.

[57] Luo, Y., Koles, K., Vorndam, W., Haltiwanger, R. S., Panin, V. M., Protein O-fucosyltransferase 2 adds O-fucose to thrombospondin type 1 repeats. J. Biol. Chem. 2006, 281, 9393-9399.

[58] Bhakta, M. S., Segal, D. J., The generation of zinc finger proteins by modular assembly. Methods Mol. Biol. 2010, 649, 3-30.

[59] Reyon, D., Tsai, S. Q., Khayter, C., Foden, J. A., et al., FLASH assembly of TALENs for high-throughput genome editing. Nat. Biotechnol. 2012, 30, 460-465.

[60] Schmid-Burgk, J. L., Schmidt, T., Kaiser, V., Honing, K., Hornung, V., A ligation-independent cloning technique for high-throughput assembly of transcription activator-like effector genes. Nat. Biotechnol. 2013, 31, 76-81.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of reducing batch-to-batch variation or increasing homogeneity between batches in the production of a recombinantly expressed polypeptide, the method comprising expressing the polypeptide in a Chinese hamster ovary (CHO) cell comprising reduced UDP-galactose transporter activity compared to a wild-type CHO cell.

2. A method according to claim 1, in which the CHO cell comprises a loss of function mutation in a UDP-galactose transporter gene (Slc35a2), for example:
(a) comprising a T insertion at position 955 of the Slc35a2 open reading frame;
(b) in which both alleles of a UDP-galactose transporter gene comprise a loss of function mutation; or
(c) in which the CHO cell is comprised in a CHO-gmt2 cell line (deposited on 21 Oct. 2014 at the American Type Culture Collection (ATCC), P.O. Box 1549, Manassas, Va. 20108, United States of America under the Budapest Treaty as accession number PTA-121624).

3. A method according to claim 1, in which the CHO cell further comprises reduced GDP-fucose transporter activity, for example:
(a) in which the CHO cell is capable of expressing recombinant antibodies for example tumour/cancer-targeting antibodies with enhanced antibody-dependent cellular cytotoxicity (ADCC);
(b) in which the CHO cell comprises a loss of function mutation in a GDP-fucose transporter gene (Slc35c1 or Slc35c2);
(c) in which both alleles of a GDP-fucose transporter gene comprise a loss of function mutation;
(d) a 3-nucleotide (GTA) insertion or a 4-nucleotide insertion at position 411 of Slc35c1; or
(e) in which the CHO cell is comprised in a CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

4. A method according to claim 1, in which:
(a) homogeneity is assayed by detecting the number of peaks in a liquid chromatogram of N-glycans of a recombinant polypeptide expressed by a CHO cell comprising reduced UDP-galactose transporter activity, as compared to a control comprising a liquid chromatogram of N-glycans of recombinant polypeptide expressed by wild type CHO-K1;
(b) a increase in homogeneity is detected as a reduction in the number of peaks as assayed in (a) above, for example to one peak compared to 3 peaks in the control;
(c) homogeneity is assayed by measuring the area under the peak of the major product (i.e., G0F) in a liquid chromatogram of N-glycans of a recombinantly expressed polypeptide as a percentage of the total area under the curve; or
(d) batch to batch variation is assayed by determining the ratio of the respective areas under the peaks of the products in a liquid chromatogram of N-glycans of a recombinantly expressed polypeptide.

5. A method according to claim 1, in which batch-to-batch variation is reduced and/or homogeneity is increased by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more or 95% or more.

6. A method according to claim 1, in which batch-to-batch variation or homogeneity or both are measured using mass spectrometry or liquid chromatography, for example liquid chromatography mass spectrometry (LC-MS) or ultra-high performance liquid chromatography (UHPLC).

7. A method according to claim 1, in which the recombinantly expressed polypeptide comprises predominantly G0F, for example with low levels or no G1F or G2F N-glycans.

8. A method according to claim 1, in which the polypeptide comprises erythropoietin-Fc fusion polypeptide (EPO-Fc), MUC1-Fc fusion polypeptide, an antibody, anti-HER2 antibody (Herceptin), Anti-CD20 antibody (Rituxan or GA101), IgG1, IgG2, IgG3 or IgG4.

9. A method according to claim 1, in which the CHO cell or cell line is adapted to suspension culture in a serum-free medium.

10. A method of production of a recombinantly expressed polypeptide with reduced batch-to-batch variation or increased homogeneity between batches, the method comprising a method according to claim 1.

11. A method according to claim 1, in which the method further comprises allowing the polypeptide to be expressed from the CHO cell or a descendent thereof and purifying the polypeptide.

12. (canceled)

13. A method comprising expressing a recombinant polypeptide in a Chinese hamster ovary (CHO) cell which has reduced UDP-galactose transporter activity compared to a wild-type cell and detecting reduced batch-to-batch variation or increased homogeneity between batches, or both.

14. A recombinant polypeptide produced by the method of claim 1.

15. A plurality of batches of recombinant polypeptide each according to claim 14, in which the batch-to-batch variation between the batches is reduced or homogeneity between the batches is increased, when compared to batches of recombinant polypeptide produced by wild-type CHO cells.

16. A method of producing a Chinese hamster ovary (CHO) cell suitable for recombinant polypeptide expression with reduced batch-to-batch variation or increased homogeneity, or both, the method comprising reducing UDP-galactose transporter activity in or of the cell, for example by introducing a loss of function mutation in a UDP-galactose transporter gene (Slc35a2).

17. A Chinese hamster ovary (CHO) cell comprising reduced UDP-galactose transporter activity and reduced GDP-fucose transporter activity.

18. A CHO cell according to claim 17 which is capable of expressing recombinant antibodies for example tumour/cancer-targeting antibodies with enhanced antibody-dependent cellular cytotoxicity (ADCC) and reduced batch-to-batch variation or increased homogeneity between batches.

19. A CHO cell according to claim 17 comprising a loss of function mutation in a UDP-galactose transporter gene (Slc35a2) and a loss of function mutation in a GDP-fucose transporter gene (Slc35c1 or Slc35c2).

20. A CHO cell according to claim 17, comprising a T insertion at position 955 of the Slc35a2 open reading frame and a 3-nucleotide (GTA) insertion or a 4-nucleotide insertion at position 411 of Slc35c1.

21. A CHO cell according to claim 17, in which the CHO cell is comprised in a CHO-gmt9 cell line (deposited on 21 Oct. 2014 at the American Type Culture Collection (ATCC), P.O. Box 1549, Manassas, Va. 20108, United States of America under the Budapest Treaty as accession number PTA-121625).

22. A CHO cell according to claim 17, which is or has been adapted to suspension culture in a serum-free medium.

23. Use of a CHO cell according to claim 17, in a method of expression of a recombinant polypeptide.
